(19) United States
(12) Patent Application Publication
(10) Pub. No.: US 2023/0058146 A1
(43) Pub. Date: Feb. 23, 2023

(54) METHOD OF DIAGNOSIS, PROGNOSIS, AND TREATMENT OF VITAMIN D DEFICIENCY AND VITAMIN D IMBALANCE AND DISEASES AND DISORDERS ASSOCIATED THEREOF

(71) Applicant: Temple University-Of The Commonwealth System of Higher Education, Philadelphia, PA (US)

(72) Inventor: Robin Taylor Wilson, Philadelphia, PA (US)

(21) Appl. No.: 17/817,696

(22) Filed: Aug. 5, 2022

Related U.S. Application Data
(60) Provisional application No. 63/230,245, filed on Aug. 6, 2021.

(51) Int. Cl. G01N 33/82; A61K 31/592; A61K 31/593; A61K 45/06; G01N 27/62

(52) U.S. Cl. CPC G01N 33/82; A61K 31/592; A61K 31/593; A61K 45/06; G01N 27/62

(57) ABSTRACT

The present invention relates to compositions and methods relating to biomarkers (e.g., calcitriol (i.e., 1,25(OH)₂D₃) to 24,25-dihydroxyvitamin D₃ (i.e., 24,25(OH)₂D₃) to calcifediol (i.e., 25(OH)D₃) proportion ratio (C24CPR)) that can be used for detection and treatment assessment of vitamin D deficiency or vitamin D imbalance and/or diseases or disorders associated with vitamin D deficiency or vitamin D imbalance, such as cancer, in a subject in need thereof. The present invention also provides methods of diagnosing vitamin D deficiency or vitamin D imbalance and/or diseases or disorders associated with vitamin D deficiency or vitamin D imbalance and distinguishing between different types of diseases or disorders associated with vitamin D deficiency or vitamin D imbalance (e.g., cancer vs autoimmune disease or disorder).

### CROSS-REFERENCE TO RELATED APPLICATIONS

This application claims priority to and the benefit of U.S. Provisional Application No. 63/230,245, filed Aug. 6, 2021, the disclosure of which is incorporated herein by reference in its entirety.

### STATEMENT REGARDING FEDERALLY SPONSORED RESEARCH OR DEVELOPMENT

This invention was made with government support under CA120092-01A2 awarded by the National Institutes of Health. The government has certain rights in the invention.

### BACKGROUND OF THE INVENTION

In considering harms and benefits of vitamin D screening among asymptomatic adults, the US Preventive Services Task Force (USPSTF) concluded that there was ‘adequate evidence not to recommend’ use of the 25(OH)D (25-hydroxyvitamin D) blood biomarker as a screening tool for the treatment of asymptomatic vitamin D deficiency—particularly for the prevention of cancer, type 2 diabetes mellitus, total mortality, or fracture risk (Kahwati L C, et al. Screening for Vitamin D Deficiency in Adults: An Evidence Review for the U.S. Preventive Services Task Force [Internet]. Rockville (Md.): Agency for Healthcare Research and Quality (US); 2021 April (Evidence Synthesis, No. 201.) ncbi.nlm.nih.gov/books/NBK569723/; LeFevre M L et al., 2015, Ann Intern Med, 162:133-140). Consistent with this conclusion, large Mendelian randomization studies report no association for genetically-predicted 25(OH)D concentrations with risk for either low bone mineral density, fracture (Trajanoska K et al., 2018, Curr Osteoporos Rep, 16:531-540), or cancers of the colon or prostate (He Y et al., 2018, BMC Med, 16:142; Dimitrakopoulou V I et al., 2017, Am J Epidemiol, 185:452-464). Several alternative blood biomarkers of vitamin D status have been proposed, and are under present consideration by the USPSTF, including 1,25(OH)₂D₃ (active form of vitamin D, calcitriol, 1,25-dihydroxycholecalciferol), other metabolite ratios and/or proportion of free versus bound to the vitamin D binding protein (Lauridsen A L et al., 2005, Calcif Tissue Int, 77:15-22; Bosworth C et al., 2013, Semin Nephrol, 33:158-168; Powe C E et al., 2013, N Engl J Med, 369:1991-2000; Pasquali M et al., 2015, BBA Clin, 3:251-256; Müller M J et al., 2015, Clin Chem, 61:1033-1048).

The 1,25(OH)₂D₃ metabolite (aka calcitriol) is known as the active form of vitamin D due to its influence on gene expression via its interaction with the vitamin D receptor (VDR), a nuclear hormone receptor. Following binding to VDR, calcitriol induces migration of VDR from the cytosol to the nucleus (Miyauchi Y et al., 2005, J Biol Chem, 280:40901-40908) where it binds to DNA and regulates gene expression (Carlberg C et al., 2013, Steroids, 78:127-136). The actions of VDR are predominantly, but not exclusively, dependent on the availability of 1,25(OH)₂D₃ (Pike J W et al., 2017, Endocrinol Metab Clin North Am, 46:815-843). In contrast, while 25(OH)D₃ can bind to VDR, it does so with lower affinity (Bukuroshi P et al., 2018, Biochem Pharmacol, 155:547-561). In spite of a substantial mechanistic literature reporting significant anti-cancer (i.e., apoptotosis) properties of 1,25(OH)₂D₃, only a handful of epidemiologic studies have been conducted. Low 1,25(OH)₂D₃ blood concentrations are associated with increased risk of colorectal adenomas (Platz E A et al., 2000, Cancer Epidemiol Biomarkers Prev, 9:1059-1065), metabolic syndrome (Bea J W et al., 2015, Metabolism, 64:447-459), and chronic pain (Hirani V et al., 2015, Journals of Gerontology Series A: Biological Sciences & Medical Sciences, 70:385-393), as well as low physical activity (Kohler L N et al., 2017, J Nutr, 147:421-429), and decline in renal function (Selamet U et al., 2018, Am J Kidney Dis, 72:419-428). Significant interactions with 1,25(OH)₂D₃ and 25(OH)D blood concentrations (Feskanich D et al., 2014, Cancer Epidemiol Biomarkers Prev, 13:1502-1508) including VDR (Mikhak B et al., 2007, Prostate, 67:911-923) on colon and prostate cancer, respectively, have been reported.

Thus, there is a need in the art for improved methods of diagnosis and prognosis of vitamin D deficiency or vitamin D imbalance as well as diseases or disorders associated with vitamin D deficiency or vitamin D imbalance in a subject that monitors levels of biomarkers associated with the vitamin D deficiency or vitamin D imbalance as well as diseases and disorders associated with vitamin D deficiency or vitamin D imbalance. Furthermore, there is also a need in the art for improved methods of treating said vitamin D deficiency or vitamin D imbalance and said diseases and disorders associated with vitamin D deficiency or vitamin D imbalance. The present invention satisfies this unmet need.

### SUMMARY OF THE INVENTION

In various aspects, the present invention provides a method of distinguishing a subject with a disease or disorder associated with vitamin D deficiency or vitamin D imbalance from a subject without disease or disorder associated with vitamin D deficiency or vitamin D imbalance. In some embodiments, the method comprises: a. obtaining a biological sample from a test subject; b. analyzing the biological sample with an assay that specifically detects at least one biomarker, wherein the at least one biomarker is calcitriol to 24,25-dihydroxyvitamin D to calcifediol proportion ratio (C24CPR); c. detecting the level of the at least one biomarker in the biological sample of the subject; d. comparing the level of the at least one biomarker in the biological sample to a comparator of the at least one biomarker; e. determining that the subject has the disease or disorder associated with vitamin D deficiency or vitamin D imbalance when the at least one biomarker is differentially expressed in the biological sample as compared to the comparator; and f. administering a treatment of the disease or disorder associated with vitamin D deficiency or vitamin D imbalance to the subject determined to have the disease or disorder associated with vitamin D deficiency or vitamin D imbalance.

In various aspects, the present invention provides a method of assessing an effectiveness of treating a disease or disorder associated with vitamin D deficiency or vitamin D imbalance in a subject. In some embodiments, the method comprises: a. obtaining a biological sample from a test subject; b. analyzing the biological sample with an assay that specifically detects at least one biomarker, wherein the at least one biomarker is C24CPR; c. detecting the level of the at least one biomarker in the biological sample of the subject; d. comparing the level of the at least one biomarker in the biological sample to a comparator of the at least one biomarker; and e. determining that the subject has disease or disorder associated with vitamin D deficiency or vitamin D imbalance when the at least one biomarker is differentially expressed in the biological sample as compared to the comparator; and f administering a treatment of the disease or disorder associated with vitamin D deficiency or vitamin D imbalance to the subject.

In other aspects, the present invention provides a method of preventing a disease or disorder associated with vitamin D deficiency or vitamin D imbalance in a subject in need thereof. In some embodiments, the method comprises: a. obtaining a biological sample from a test subject; b. analyzing the biological sample with an assay that specifically detects at least one biomarker, wherein the at least one biomarker is C24CPR; c. detecting the level of the at least one biomarker in the biological sample of the subject; d. comparing the level of the at least one biomarker in the biological sample to a comparator of the at least one biomarker; and e. determining that the subject is at risk of developing the disease or disorder associated with vitamin D deficiency or vitamin D imbalance when the at least one biomarker is differentially expressed in the biological sample as compared to the comparator.

In other aspects, the present invention provides a method of detecting a vitamin D deficiency or vitamin D imbalance in a subject. In some embodiments, the method comprises: a. obtaining a biological sample from a test subject; b. analyzing the biological sample with an assay that specifically detects at least one biomarker, wherein the at least one biomarker is C24CPR; c. detecting the level of the at least one biomarker in the biological sample of the subject; d. comparing the level of the at least one biomarker in the biological sample to a comparator of the at least one biomarker; and e. determining that the subject has vitamin D deficiency or vitamin D imbalance when the at least one biomarker is differentially expressed in the biological sample as compared to the comparator.

In other aspects, the present invention provides a method of assessing a risk of hypertension in a subject. In some embodiments, the method comprises: a. obtaining a biological sample from a test subject; b. analyzing the biological sample with an assay that specifically detects at least one biomarker, wherein the at least one biomarker is C24CPR; c. detecting the level of the at least one biomarker in the biological sample of the subject; d. comparing the level of the at least one biomarker in the biological sample to a comparator of the at least one biomarker; and e. determining that the subject is at a risk of hypertension when the at least one biomarker is differentially expressed in the biological sample as compared to the comparator.

In some embodiments, the disease or disorder associated with vitamin D deficiency or vitamin D imbalance is selected from a cancer, autoimmune disease or disorder, diabetes, adenoma, metabolic syndrome, chronic pain, decline in renal function, or any combination thereof. In some embodiments, the disease or disorder associated with vitamin D deficiency or vitamin D imbalance is selected from a renal cell cancer, prostate cancer, colon cancer, colorectal cancer, colorectal adenoma, type 2 diabetes, or any combination thereof.

In some embodiments, the biological sample comprises a biological tissue of the subject, a blood sample of the subject, a bodily fluid sample of the subject, a fecal sample of the subject, a plasma sample of the subject, a saliva sample of the subject, a urine sample of the subject, or any combination thereof.

In some embodiments, the assay is selected from mass spectrometry (MS), liquid chromatography (LC), liquid chromatography-mass spectrometry (LC-MS), targeted liquid chromatography-tandem mass spectrometry (LC-MS/MS), high-performance liquid chromatography (HPLC), ultra-performance liquid chromatography (UPLC), ultra-high-performance liquid chromatography (UHPLC), gas chromatography (GC), gas chromatography-mass spectrometry (GC-MS), globally optimized targeted mass spectrometry, targeted assay of about 200 metabolites, aqueous global profiling, liquid global profiling, GC-MS profiling, GC-MS flux analysis, carnitine analysis, lipid targeted analysis, quantitative lipid targeted analysis, tryptophan analysis, absolute quantification, multivariate statistical analysis, dynamic light scattering (DLS), nuclear magnetic resonance (NMR) spectroscopy, ultraviolet-visible (UV/Vis) spectroscopy, infrared (IR) spectroscopy, Raman spectroscopy, or any combination thereof.

In some embodiments, the method comprises using a multi-dimensional non-linear algorithm to determine if the level of a set of biomarkers in the biological sample is statistically different as compared to the comparator.

In some embodiments, the subject is determined to have disease or disorder associated with vitamin D deficiency or vitamin D imbalance when the level of the at least one biomarker in the biological sample is decreased as compared to the comparator.

In some embodiments, the prevention or treatment is determined based on the level of the at least one biomarker in a subject. In some embodiments, the prevention or treatment is adjusted based on the level of the at least one biomarker in a subject. In some embodiments, the prevention or treatment comprises administering a therapeutically effective amount of at least one selected from the group consisting of agonist of calcitriol, agonist of 24,25-dihydroxyvitamin D, agonist of calcifediol, antagonist of calcitriol, antagonist of 24,25-dihydroxyvitamin D, antagonist of calcifediol, and any combination thereof.

In some embodiments, the subject has at least one genetic variation associated with vitamin D metabolite. In some embodiments, the at least one genetic variation associated with vitamin D metabolite is selected from one or more variations in CYP3A43, one or more variations in FGF23, one or more variations in KL, one or more variations in VDR, one or more variations in CALB1, one or more variations in CYP27B1, one or more variations in GC, one or more variations in PPARGC1A, or any combination thereof. In some embodiments, the at least one genetic variation associated with vitamin D metabolite is selected from one or more variations in CYP3A43, one or more variations in FGF23, one or more variations in KL, one or more variations in VDR, or any combination thereof.

In some embodiments, the subject is a woman.

In some embodiments, the comparator is specific to genetic ancestry-based ranges identified in a healthy subject.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts a schematic representation of results demonstrating that West African genetic ancestry proportion (WAGAP) accounts for the greatest proportion of the variability in the calcitriol (i.e., 1,25(OH)₂D₃) to 24,25-dihydroxyvitamin D₃ (i.e., 24,25(OH)₂D₃) to calcifediol (i.e., 25(OH)D₃) proportion ratio (C24CPR) biomarker, compared with 1,25(OH)₂D₃ (28.2% versus 0.06%, respectively). This indicated that WAGAP ranges can be developed for clinical normal ranges of the biomarker. Overall, a greater proportion of the C24CPR biomarker was explained by known genetic and environmental factors, compared with 1,25(OH)₂D₃ (49.5% versus 37.4%, respectively). This indicated that while more of the variability in C24CPR biomarker was explained, there is still more to be known. Furthermore, the pattern of gene variants associated with the C24CPR biomarker were not the same as the pattern of gene variants associated with 1,25(OH)₂D₃. This indicated that the C24CPR biomarker was capturing a biological status that was different from 1,25(OH)₂D₃.

FIG. 2 depicts representative osteoporosis risk factor models adjusted for age, sex, systolic blood pressure (mmHg), pack years of smoking, tanning bed use in the past 30 days, West African genetic ancestry proportion, current oral contraceptive and medroxyprogesterone acetate use, vitamin D intake from diet and supplements (including multi-vitamins), days from summer solstice at time of blood draw, time of day of blood draw, body mass index class, and other genetic polymorphisms with listed values in the table (including the interaction term).

FIG. 3 depicts representative results demonstrating concordance correlation coefficient (CCC) plot comparing the calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR) to the crude 1,25(OH)₂D₃ to 24,25(OH)₂D₃ concentration ratio. The line of perfect agreement between C24CPR and the crude ratio of the dihydroxy metabolites is represented by the 45 degree diagonal line. The CCC for the figure is 0.49, (95% confidence interval: 0.45, 0.53). The non-log transformed CCC is 0.22 (0.20-0.24). C24CPR calculated as [(1,25(OH)₂D₃ pmol/L*1000)/((1,25(OH)₂D₃ pmol/L*1000)+25(OH)D₃ nmol/L)]/[(24,25(OH)₂D₃ nmol/L)/((24,25(OH)₂D₃ nmol/L)+25(OH)D₃ nmol/L)]. Perfect agreement of the CCC is a value of 1.0.

FIG. 4 depicts representative results demonstrating the distribution of vitamin D biomarkers by race/ethnicity, sex, and oral contraceptive use. The coefficient of variation (CV %) for C24CPR is lower than a simple ratio of 1,25(OH)₂D₃ to 24,25(OH)₂D₃, regardless of sex, race/ethnicity or oral contraceptive use (women only). The skewness of the distribution of C24CPR is comparable to a simple ratio of 1,25(OH)₂D₃ to 24,25(OH)₂D₃, regardless of sex, race/ethnicity or oral contraceptive use (women only).

FIG. 5, comprising FIG. 5A through FIG. 5D, depicts representative log linear plots of plasma calcitriol (1,25(OH)₂D₃) and plasma calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR) in relation to the skin melanin index (SMI) and West African Genetic Ancestry Proportion (WAGAP) among healthy African American a European American participants. LED light reflectance was used to estimate skin melanin index of the upper inner arm using the DermaSpectrometer II. FIG. 5A depicts representative log plasma 1,25(OH)₂D₃ Spearman's rho correlations with SMI that are 0.05 (95% confidence interval (CI): −0.05, 0.15). FIG. 5B depicts representative log plasma 1,25(OH)₂D₃ Spearman's rho correlations with WAGAP that are 0.10 (95% confidence interval (CI): (0.00, 0.20). FIG. 5C depicts log plasma C24CPR Spearman's rho correlations with SMI that are 0.49 (0.41, 0.56). FIG. 5D depicts log plasma C24CPR Spearman's rho correlations with WAGAP that are 0.51 (0.43, 0.58).

FIG. 6 depicts representative pairwise D′ and r² linkage disequilibrium values between single nucleotide polymorphisms of interest and Spearman's correlation with West African Genetic Ancestry proportion, African American (N=188) matched with European American (N=188) participants, Central Pennsylvania *. The direction of Spearman's correlation (positive) is in correlation with the least frequent genotype for total study population, including both African American and European American. Figures not rounding to two digits are truncated to 0.00.

FIG. 7 depicts the Location of rs115631506 (at Chr12:57,767,598) in the CYP27B1 promoter and the concurrent binding location of transcription factors REX1 and NeuroD1 as identified with ChIP-seq experiments in multiple cell lines using the Gene Transcription Regulation Database (gtrd.biouml.org/).

FIG. 8 depicts representative genomic context of CYP3A7 rs2257401 variant. The location of the CYP3A7 rs2257401 variant is indicated by the light blue vertical stripe running through the figure. Genomic tracks from top to bottom include the scale for Chromosome 7, vertebrate conservation score (cons 100 verts), gene names and orientation, 1000 Genomes Phase 3 Integrated Variant Calls from International Genome Sample Resource (IGSR) including insertion deletion (indels), missense polymorphisms from the Common 1000 Genomes Phase 3 with minor allele frequency >1%, and Structural Variation. All missense single nucleotide polymorphic variants (dbSNP release 153) in the region appear in red with rs number. The length of the solid black vertical bars approximate local haplotype blocks centered on the CYP3A7 rs2257401 variant and estimated using parent-child trios. Structural DNA sequence gains as mapped to this chromosome appear in blue horizontal bars and structural DNA sequence losses appear in as red horizontal bars. Source: UCSC Genome Browser, Jan. 31, 2020.

FIG. 9 depicts representative receiver-operator characteristics for prediction of hypertension with respect to ancestry-specific quartiles of C24CPR among women using oral contraceptives. Ancestry-specific quartiles among normotensive individuals determined using West African Genetic Ancestry proportion (>0.50 and ≤0.50) among 238 healthy individuals self-identifying with at least 50% African Ancestry or at least 50% Caucasian ancestry. Rank ordering of the quartiles reflects the lower risk in quartile 2 (Q2), compared with quartiles 1 (Q1), 3 (Q3), and 4 (Q4), respectively, and ordered as Q2, Q1, Q3, and Q4. Area Under the Curve=0.691.

FIG. 10 depicts the correlation between a Vitamin D Pathway Genetic Risk Score (VDGRS) and the failure of patient lymphocytes to repair double-strand DNA breakage (DSB). DSB was measured by the fold-induction of gamma-H2AX following laboratory culturing of patient lymphocytes with a DNA damaging agent (aphidicolin) among patients with young age at onset (“early onset”) diagnosis of renal cell cancer (eoRCC).

FIG. 11 depicts the association between the VDGRS and C24CPR, 25(OH)D₃ and 1,25(OH)D₃. Of the three vitamin D biomarkers, C24CPR is the only one that is significantly associated with the VDGRS in both bivariate and multivariable adjusted models. C24CPR decreases in a monotonic fashion with increasing number of VDGRS risk alleles.

### DETAILED DESCRIPTION

The present invention is based in part on the discovery that the calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR) is an effective predictor of vitamin D deficiency or vitamin D imbalance in a subject. Thus, the present invention relates to compositions and methods relating to biomarkers (e.g., one or more vitamin D metabolites or a combination thereof) that can be used for identifying, diagnosing, assessing the prognosis of vitamin D deficiency or vitamin D imbalance as well as diseases or disorders associated with vitamin D deficiency or vitamin D imbalance (e.g., cancer, autoimmune disease or disorder, chronic pain, diabetes, chronic pain) in a subject. In one embodiment, the subject has a genetic variation associated with one or more vitamin D-pathway genes. As described herein, a decreased calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR) is demonstrated to be a useful diagnostic and prognostic biomarker for vitamin D deficiency or vitamin D imbalance and diseases or disorders associated with vitamin D deficiency or vitamin D imbalance (e.g., cancer, autoimmune disease or disorder, chronic pain, diabetes, chronic pain). The present invention further provides methods relating to the biomarkers (e.g., one or more vitamin D metabolites or a combination thereof) of the invention that can be used to establish and evaluate treatment plans for a subject having a disease or disorder associated with vitamin D deficiency or vitamin D imbalance (e.g., cancer, autoimmune disease or disorder, chronic pain, diabetes, chronic pain).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A disease or disorder is “alleviated” if the severity of a sign or symptom of the disease or disorder, the frequency with which such a sign or symptom is experienced by a patient, or both, is reduced.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

As used herein, the term “diagnosis” refers to the determination of the presence of a disease or disorder. In some embodiments of the present invention, methods for making a diagnosis are provided which permit determination of the presence of a particular disease or disorder.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

As used herein, “polymorph” refers to crystalline forms having the same chemical composition but different spatial arrangements of the molecules, atoms, and/or ions forming the crystal.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the patient is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

The term “nutritional composition” may be a food product intended for human consumption, for example, a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a microwave or an oven, a ready-to-eat product, a fast food or a nutritional formula.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “genome” as used herein is all the genetic material in the chromosomes of an organism (i.e., subject). DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information a subject (e.g., individual) carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “allele” as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

“Instructional material”, as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

Assays for amplification of the known sequence are also disclosed. For example, primers for PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome, such as those coding for a protein or RNA of interest; or a conserved region from multiple genomes; or multiple genomes.

The term “amplification” refers to the operation by which the number of copies of a target nucleotide sequence present in a sample is multiplied.

As used herein, an “immunoassay” refers to any binding assay that uses an antibody capable of binding specifically to a target molecule to detect and quantify the target molecule.

The term “specifically binds”, as used herein with respect to an antibody, is meant for an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, or a protein with a sequence of DNA, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, adenine, and guanine, respectively. Indeed, the present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

As used herein, the term “polymerase chain reaction” (“PCR”) refers to the method of K. B. Mullis, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers are then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. As used herein, the terms “PCR product”, “PCR fragment”, “amplification product”, or “amplicon” refer to the resultant mixture of compounds after two or more cycles of the PCR steps of denaturation, annealing and extension are complete. These terms encompass the case where there has been amplification of one or more segments of one or more target sequences.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences.

As used herein, the term “oligonucleotide” refers to an oligomer or polymer of either ribonucleic acid (RNA) or deoxyribonucleic acid (DNA), as well as non-naturally occurring oligonucleotides. Non-naturally occurring oligonucleotides are oligomers or polymers which contain nucleobase sequences which do not occur in nature, or species which contain functional equivalents of naturally occurring nucleobases, sugars, or inter-sugar linkages, like aptamers, spiegelmers, peptide nucleid acids (PNA), threose nucleic acids (TNA), locked nucleic acids (LNA), or glycerol nucleic acids (GNA). This term includes oligomers that contain the naturally occurring nucleic acid nucleobases adenine (A), guanine (G), thymine (T), cytosine (C) and uracil (U), as well as oligomers that contain base analogs or modified nucleobases. Therefore the person skilled in the art understands that the term “oligonucleotide” comprises, but is not limited to, RNA, DNA and mixed oligonucleotides, antisense oligonucleotides, short interfering RNA (siRNA), microRNAs (miRNAs), aptamers, and also spiegelmers. Oligonucleotides can be derived from a variety of natural sources, such as viral, bacterial and eukaryotic DNAs and RNAs. Other oligonucleotides can be derived from synthetic sources and include any of the multiple oligonucleotides that are being manufactured for use as research reagents, diagnostic agents or potential and definite therapeutic agents. The term includes oligomers comprising of a single strand nucleic acid or a double strand nucleic acid.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based in part on the discovery that the calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR) is an effective predictor of vitamin D deficiency or vitamin D imbalance in a subject. Thus, the present invention relates to compositions and methods relating to biomarkers (e.g., one or more vitamin D metabolites or a combination thereof) that can be used for identifying, diagnosing, assessing the prognosis of vitamin D deficiency or vitamin D imbalance as well as diseases or disorders associated with vitamin D deficiency or vitamin D imbalance (e.g., cancer, autoimmune disease or disorder, chronic pain, diabetes, chronic pain) in a subject. In one embodiment, the subject has a genetic variation associated with one or more vitamin D-pathway genes. As described herein, a decreased calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR) is demonstrated to be a useful diagnostic and prognostic biomarker for vitamin D deficiency or vitamin D imbalance and diseases or disorders associated with vitamin D deficiency or vitamin D imbalance (e.g., cancer, autoimmune disease or disorder, chronic pain, diabetes, chronic pain). The present invention further provides methods relating to the biomarkers (e.g., one or more vitamin D metabolites or a combination thereof) of the invention that can be used to establish and evaluate treatment plans for a subject having a disease or disorder associated with vitamin D deficiency or vitamin D imbalance (e.g., cancer, autoimmune disease or disorder, chronic pain, diabetes, chronic pain).

### Methods of Diagnosing and Assessing the Prognosis

In one aspect, the present invention provides a method for diagnosing or assessing the prognosis of vitamin D deficiency or vitamin D imbalance in a subject in need thereof. In one aspect, the present invention provides a method for diagnosing or assessing the prognosis of at least one disease or disorder associated with vitamin D deficiency or vitamin D imbalance in a subject in need thereof. In one aspect, the present invention also provides methods for distinguishing a subject with at least one disease or disorder associated with vitamin D deficiency or vitamin D imbalance from a subject without disease or disorder associated with vitamin D deficiency or vitamin D imbalance. In one aspect, the present invention further provides methods relating to the biomarker of the present invention (e.g., one or more vitamin D metabolites or a combination thereof, such as calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR)) that can be used to establish and evaluate treatment plans for a subject with vitamin D deficiency or vitamin D imbalance and/or at least one disease or disorder associated with vitamin D deficiency or vitamin D imbalance.

In one embodiment, the method comprises obtaining a biological sample from a test subject. In one embodiment, the method comprises analyzing the biological sample with an assay that specifically detects at least one biomarker. In one embodiment, the method comprises detecting the level of the at least one biomarker in the biological sample of the subject. In one embodiment, the method comprises comparing the level of the at least one biomarker in the biological sample to a comparator. In one embodiment, the method comprises determining that the subject has at least one disease or disorder associated with vitamin D deficiency or vitamin D imbalance when the at least one biomarker is differentially expressed in the biological sample as compared to the comparator. In one embodiment, the method comprises administering a treatment of the disease or disorder associated with vitamin D deficiency or vitamin D imbalance to the subject.

In one embodiment, the method comprises at least one biomarker. In one embodiment, the method comprises one or more biomarkers. In one embodiment, the method comprises two or more biomarkers. In one embodiment, the method comprises three or more biomarkers. In one embodiment, the method comprises four or more biomarkers. In one embodiment, the method comprises five or more biomarkers. In one embodiment, the method comprises six or more biomarkers. In one embodiment, the method comprises seven or more biomarkers. In one embodiment, the method comprises eight or more biomarkers. In one embodiment, the method comprises nine or more biomarkers. In one embodiment, the method comprises ten or more biomarkers. In one embodiment, the method comprises eleven or more biomarkers. In one embodiment, the method comprises twelve or more biomarkers. In one embodiment, the method comprises thirteen or more biomarkers. In one embodiment, the method comprises fourteen or more biomarkers. In one embodiment, the method comprises fifteen or more biomarkers. In one embodiment, the method comprises sixteen or more biomarkers. In one embodiment, the method comprises seventeen or more biomarkers. In one embodiment, the method comprises eighteen or more biomarkers. In one embodiment, the method comprises nineteen or more biomarkers. In one embodiment, the method comprises twenty or more biomarkers.

In some embodiments, the present invention is at least one biomarker for identification, diagnosis, assessment of prognosis, and/or treatment evaluation of disease or disorder associated with vitamin D deficiency or vitamin D imbalance in a subject.

In one embodiment, the present invention is at least one biomarker for distinguishing a subject with a disease or disorder associated with vitamin D deficiency or vitamin D imbalance from a subject without disease or disorder associated with vitamin D deficiency or vitamin D imbalance.

In one embodiment, the present invention is at least one biomarker for assessing an effectiveness of treating a disease or disorder associated with vitamin D deficiency or vitamin D imbalance in a subject.

In one embodiment, the present invention is at least one biomarker for prevention of a disease or disorder associated with vitamin D deficiency or vitamin D imbalance in a subject in need thereof.

In one embodiment, the present invention is at least one biomarker for detection of vitamin D deficiency or vitamin D imbalance in a subject.

In one embodiment, the biomarker is calcitriol (i.e., 1,25(OH)₂D₃). In one embodiment, the biomarker is 24,25-dihydroxyvitamin D3 (i.e., 24,25(OH)₂D₃). In one embodiment, the biomarker is calcifediol (i.e., 25(OH)D₃). In some embodiments, the biomarker is a set of biomarkers comprising one or more of calcitriol (i.e., 1,25(OH)₂D₃), 24,25-dihydroxyvitamin D3 (i.e., 24,25(OH)₂D₃), and calcifediol (i.e., 25(OH)D₃). Thus, in one embodiment, the biomarker is calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR)).

In some embodiments, the biomarker is calcitriol (i.e., 1,25(OH)₂D₃), 24,25-dihydroxyvitamin D3 (i.e., 24,25(OH)₂D₃), calcifediol (i.e., 25(OH)D₃), calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR)), or any combination thereof.

In certain embodiments, the method comprises determining if the level of the relevant biomarkers is differentially expressed as compared to a comparator. In certain embodiments, the comparator may be the level (e.g., activity, amount, concentration, concentration of the ionized form, concentration of the neutral form, expression, level, etc.) of the relevant biomarkers in a subject not having vitamin D deficiency or vitamin D imbalance, a population not having vitamin D deficiency or vitamin D imbalance, or a combination thereof.

In certain embodiments, the comparator may be the level (e.g., activity, amount, concentration, concentration of the ionized form, concentration of the neutral form, expression, level, etc.) of the relevant biomarkers in a subject not having a disease or disorder associated with vitamin D deficiency or vitamin D imbalance, a population not having a disease or disorder associated with vitamin D deficiency or vitamin D imbalance, or a combination thereof. In certain embodiments, the method comprises determining if the levels of the relevant biomarkers in a sample obtained from the subject are differentially expressed as compared to the levels of the relevant biomarkers in a subject and/or population where a disease or disorder associated with vitamin D deficiency or vitamin D imbalance has not recurred.

In various embodiments, the subject is a human subject, and may be of any race, ethnicity, sex, and age.

In various embodiments, the subject has at least one genetic variation associated with vitamin D metabolite. For example, in some embodiments, the genetic variation associated with vitamin D metabolite is one or more variations in CYP3A43, one or more variations in FGF23, one or more variations in KL, one or more variations in VDR, one or more variations in CALB1, one or more variations in CYP27B1, one or more variations in GC, one or more variations in PPARGC1A, or any combination thereof. In some embodiments, the genetic variation associated with vitamin D metabolite is one or more variations in CYP3A43, one or more variations in FGF23, one or more variations in KL, one or more variations in VDR, or any combination thereof.

In various embodiments, the subject has at least one genetic variation associated with at least one biomarker of vitamin D deficiency or vitamin D imbalance. For example, in some embodiments, the genetic variation associated with at least one biomarker of vitamin D deficiency or vitamin D imbalance is one or more variations in CYP3A43, one or more variations in FGF23, one or more variations in KL, one or more variations in VDR, one or more variations in CALB1, one or more variations in CYP27B1, one or more variations in GC, one or more variations in PPARGC1A, or any combination thereof. In some embodiments, the genetic variation associated with vitamin D metabolite is one or more variations in CYP3A43, one or more variations in FGF23, one or more variations in KL, one or more variations in VDR, or any combination thereof.

In various embodiments, the comparator is the level (e.g., activity, amount, concentration, concentration of the ionized form, concentration of the neutral form, expression, level, etc.) of the relevant biomarkers in a biological sample obtained from a human subject, an average of multiple human subjects, an average of multiple human subjects living in the same region, an average of multiple human subjects with the same age, an average of multiple human subjects with the same race, an average of multiple human subjects with the same ethnicity, an average of multiple human subjects with the same sex, an average of multiple human subjects living in different regions, an average of multiple human subjects with different ages, an average of multiple human subjects with different races, an average of multiple human subjects with different ethnicities, an average of multiple human subjects with different sexes, or any combination thereof.

In one embodiment, the method comprises detecting the level of at least one biomarker in a biological sample obtained from the subject, wherein the at least one biomarker is selected from the group consisting of calcitriol, 24,25-dihydroxyvitamin D, calcifediol, calcitriol to 24,25-dihydroxyvitamin D to calcifediol proportion ratio (C24CPR)), or any combination thereof; comparing the level of the at least one biomarker in the biological sample to a comparator; and determining that the subject has a disease or disorder associated with vitamin D deficiency or vitamin D imbalance when the at least one biomarker is differentially expressed in the biological sample as compared to the comparator.

In one embodiment, the method comprises detecting the level of at least one biomarker in a biological sample obtained from the subject, wherein the at least one biomarker is selected from the group consisting of calcitriol, 24,25-dihydroxyvitamin D, calcifediol, calcitriol to 24,25-dihydroxyvitamin D to calcifediol proportion ratio (C24CPR)), or any combination thereof; comparing the level of the at least one biomarker in the biological sample to a comparator; and determining that the subject is at risk of developing a disease or disorder associated with vitamin D deficiency or vitamin D imbalance when the at least one biomarker is differentially expressed in the biological sample as compared to the comparator.

In one embodiment, the method comprises detecting the level of at least one biomarker in a biological sample obtained from the subject, wherein the at least one biomarker is selected from the group consisting of calcitriol, 24,25-dihydroxyvitamin D, calcifediol, calcitriol to 24,25-dihydroxyvitamin D to calcifediol proportion ratio (C24CPR)), or any combination thereof; comparing the level of the at least one biomarker in the biological sample to a comparator; and determining that the subject has vitamin D deficiency or vitamin D imbalance when the at least one biomarker is differentially expressed in the biological sample as compared to the comparator.

In one embodiment, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of calcitriol is increased in the biological sample as compared to a comparator.

In one embodiment, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of calcitriol is decreased in the biological sample as compared to a comparator.

In one embodiment, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of 24,25-dihydroxyvitamin D is increased in the biological sample as compared to a comparator.

In one embodiment, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of 24,25-dihydroxyvitamin D is decreased in the biological sample as compared to a comparator.

In one embodiment, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of calcifediol is decreased in the biological sample as compared to a comparator.

In one embodiment, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of calcifediol is increased in the biological sample as compared to a comparator.

In one embodiment, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the proportion ratio of calcitriol to 24,25-dihydroxyvitamin D to calcifediol (C24CPR)) is decreased in the biological sample as compared to a comparator.

In one embodiment, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the proportion ratio of calcitriol to 24,25-dihydroxyvitamin D to calcifediol (C24CPR)) is increased in the biological sample as compared to a comparator.

In some embodiments, the disease or disorder associated with vitamin D deficiency or vitamin D imbalance is a disease or disorder associated with an increased calcitriol level or activity, increased 24,25-dihydroxyvitamin D level or activity, decreased calcifediol level or activity, decrease calcitriol to 24,25-dihydroxyvitamin D to calcifediol proportion ratio (C24CPR)), or any combination thereof in a subject.

In some embodiments, the disease or disorder associated with vitamin D imbalance is a disease or disorder associated with an increased calcitriol level or activity, decreased 24,25-dihydroxyvitamin D level or activity, increased calcifediol level or activity, increase calcitriol to 24,25-dihydroxyvitamin D to calcifediol proportion ratio (C24CPR)), or any combination thereof in a subject.

In various embodiments of the methods of the invention, the level (e.g., activity, amount, concentration, concentration of the ionized form, concentration of the neutral form, expression, level, etc.) of calcifediol, the proportion ratio of calcitriol to 24,25-dihydroxyvitamin D to calcifediol (C24CPR)), or any combination thereof, is determined to be decreased when the level of calcifediol, the proportion ratio of calcitriol to 24,25-dihydroxyvitamin D to calcifediol (C24CPR)), or any combination thereof, in the biological sample is decreased by at least 1%, by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared to a comparator.

In various embodiments of the methods of the invention, the level (e.g., activity, amount, concentration, concentration of the ionized form, concentration of the neutral form, expression, level, etc.) of calcifediol, the proportion ratio of calcitriol to 24,25-dihydroxyvitamin D to calcifediol (C24CPR)), or any combination thereof, is determined to be decreased when the level of calcifediol, the proportion ratio of calcitriol to 24,25-dihydroxyvitamin D to calcifediol (C24CPR)), or any combination thereof, in the biological sample is decreased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared to a comparator.

In one embodiment, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the expression level of calcifediol, the proportion ratio of calcitriol to 24,25-dihydroxyvitamin D to calcifediol (C24CPR)), or any combination thereof, is decreased in the biological sample as compared to a comparator. For example, in some embodiments, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of calcifediol is decreased by at least 0.1 fold, or at least 0.18 fold. In some embodiments, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of calcifediol is decreased in a range from 0.01 fold to 10,000 fold.

In some embodiments, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the proportion ratio of calcitriol to 24,25-dihydroxyvitamin D to calcifediol (C24CPR)) is decreased by at least 0.3 fold, or at least 0.38 fold. In some embodiments, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the proportion ratio of calcitriol to 24,25-dihydroxyvitamin D to calcifediol (C24CPR)) is decreased in a range from 0.01 fold to 10,000 fold.

In various embodiments of the methods of the invention, the level (e.g., activity, amount, concentration, concentration of the ionized form, concentration of the neutral form, expression, level, etc.) of calcifediol, the proportion ratio of calcitriol to 24,25-dihydroxyvitamin D to calcifediol (C24CPR)), or any combination thereof, is determined to be increased when the level of calcifediol, the proportion ratio of calcitriol to 24,25-dihydroxyvitamin D to calcifediol (C24CPR)), or any combination thereof, in the biological sample is increased by at least 1%, by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared to a comparator.

In various embodiments of the methods of the invention, the level (e.g., activity, amount, concentration, concentration of the ionized form, concentration of the neutral form, expression, level, etc.) of calcifediol, the proportion ratio of calcitriol to 24,25-dihydroxyvitamin D to calcifediol (C24CPR)), or any combination thereof, is determined to be increased when the level of calcifediol, the proportion ratio of calcitriol to 24,25-dihydroxyvitamin D to calcifediol (C24CPR)), or any combination thereof, in the biological sample is increased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared to a comparator.

In one embodiment, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the expression level of calcifediol, the proportion ratio of calcitriol to 24,25-dihydroxyvitamin D to calcifediol (C24CPR)), or any combination thereof, is increased in the biological sample as compared to a comparator. For example, in some embodiments, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of calcifediol is increased by at least 0.1 fold, or at least 0.18 fold. In some embodiments, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of calcifediol is increased in a range from 0.01 fold to 10,000 fold.

In some embodiments, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the proportion ratio of calcitriol to 24,25-dihydroxyvitamin D to calcifediol (C24CPR)) is increased by at least 0.3 fold, or at least 0.38 fold. In some embodiments, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the proportion ratio of calcitriol to 24,25-dihydroxyvitamin D to calcifediol (C24CPR)) is increased in a range from 0.01 fold to 10,000 fold.

In various embodiments of the methods of the invention, the level (e.g., activity, amount, concentration, concentration of the ionized form, concentration of the neutral form, expression, level, etc.) of calcitriol, 24,25-dihydroxyvitamin D, or any combination thereof, is determined to be increased when the level of calcitriol, 24,25-dihydroxyvitamin D, or any combination thereof, in the biological sample is increased by at least 1%, by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared to a comparator.

In various embodiments of the methods of the invention, the level (e.g., activity, amount, concentration, concentration of the ionized form, concentration of the neutral form, expression, level, etc.) of calcitriol, 24,25-dihydroxyvitamin D, or any combination thereof, is determined to be increased when the level of calcitriol, 24,25-dihydroxyvitamin D, or any combination thereof, in the biological sample is increased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared to a comparator.

In one embodiment, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the expression level of calcitriol, 24,25-dihydroxyvitamin D, or any combination thereof, is increased in the biological sample as compared to a comparator. For example, in some embodiments, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of calcitriol is increased by at least 2 fold, at least 2.3 fold, or at least 2.31 fold. In some embodiments, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of calcitriol is increased in a range from 0.01 fold to 10,000 fold.

In some embodiments, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of 24,25-dihydroxyvitamin D is increased by at least 2 fold, at least 23 fold, or at least 203 fold. In some embodiments, a subject is identified as having a disease or disorder associated with vitamin D deficiency or vitamin D imbalance, having a vitamin D deficiency or vitamin D imbalance, being at risk of developing a disease or disorder associated with vitamin D deficiency or vitamin D imbalance, and/or being at risk of developing a vitamin D deficiency or vitamin D imbalance when the level of 24,25-dihydroxyvitamin D is increased in a range from 0.01 fold to 10,000 fold.

In various embodiments of the methods of the invention, the level (e.g., activity, amount, concentration, concentration of the ionized form, concentration of the neutral form, expression, level, etc.) of calcitriol, 24,25-dihydroxyvitamin D, or any combination thereof, is determined to be decreased when the level of calcitriol, 24,25-dihydroxyvitamin D, or any combination thereof, in the biological sample is decreased by at least 1%, by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared to a comparator.

In various embodiments of the methods of the invention, the level (e.g., activity, amount, concentration, concentration of the ionized form, concentration of the neutral form, expression, level, etc.) of calcitriol, 24,25-dihydroxyvitamin D, or any combination thereof, is determined to be decreased when the level of calcitriol, 24,25-dihydroxyvitamin D, or any combination thereof, in the biological sample is decreased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared to a comparator.

In one embodiment, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the expression level of calcitriol, 24,25-dihydroxyvitamin D, or any combination thereof, is decreased in the biological sample as compared to a comparator. For example, in some embodiments, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of calcitriol is decreased by at least 2 fold, at least 2.3 fold, or at least 2.31 fold. In some embodiments, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of calcitriol is decreased in a range from 0.01 fold to 10,000 fold.

In some embodiments, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of 24,25-dihydroxyvitamin D is decreased by at least 2 fold, at least 23 fold, or at least 203 fold. In some embodiments, a subject is identified as having a disease or disorder associated with vitamin D imbalance, having a vitamin D imbalance, being at risk of developing a disease or disorder associated with vitamin D imbalance, and/or being at risk of developing a vitamin D imbalance when the level of 24,25-dihydroxyvitamin D is decreased in a range from 0.01 fold to 10,000 fold.

In some embodiments, the disease or disorder associated with vitamin D deficiency or vitamin D imbalance is a cancer, autoimmune disease or disorder, diabetes, adenoma, metabolic syndrome, chronic pain, hypertension decline in renal function, or any combination thereof.

The following are non-limiting examples of cancers that can be treated by the disclosed methods and compositions: acute lymphoblastic leukemia; acute myeloid leukemia; adrenocortical carcinoma; adrenocortical carcinoma, childhood; appendiceal carcinoma; basal cell carcinoma; bile duct cancer, extrahepatic; bladder cancer; bone cancer; osteosarcoma and malignant fibrous histiocytoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt's lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

For example, in some embodiments, the disease or disorder associated with vitamin D deficiency or vitamin D imbalance is a renal cell cancer, renal cancer, prostate cancer, colon cancer, colorectal cancer, colorectal adenoma, type 2 diabetes, or any combination thereof.

In one aspect, the present invention relates to a method of distinguishing between a subject with a disease or disorder associated with vitamin D deficiency or vitamin D imbalance and a subject without a disease or disorder associated with vitamin D deficiency or vitamin D imbalance. In one aspect, the present invention relates to a method of distinguishing between different types of diseases or disorders associated with vitamin D deficiency or vitamin D imbalance (e.g., cancer, autoimmune disease or disorder, diabetes, adenoma, metabolic syndrome, chronic pain, decline in renal function, etc.). In one embodiment, the method distinguishes between subjects with different types of diseases or disorders associated with vitamin D deficiency or vitamin D imbalance (e.g., cancer, autoimmune disease or disorder, diabetes, adenoma, metabolic syndrome, chronic pain, decline in renal function, etc.).

For example, in one embodiment, the method distinguishes between a subject with a cancer and a subject with an autoimmune disease or disorder. In one embodiment, the method distinguishes between a subject with a cancer and a subject with an adenoma. In one embodiment, the method distinguishes between a subject with a cancer and a subject with a metabolic syndrome. In one embodiment, the method distinguishes between a subject with a cancer and a subject with a chronic pain. In one embodiment, the method distinguishes between a subject with a cancer and a subject with a decline in renal function.

In some methods of the invention, a biological sample from a subject is assessed for the level of one or more of the markers of the invention in the biological sample obtained from the patient. The level of one or more of the markers of the invention in the biological sample can be determined by assessing the amount of one or more of the biomarkers of the invention in the biological sample, the amount of activity of one or more of the biomarkers of the invention in the biological sample, the amount of concentration of one or more of the biomarkers of the invention in the biological sample, the amount of one or more of the biomarkers of the invention in the biological sample, or any combination thereof.

Biological samples may be of any biological tissue or fluid. Frequently the sample will be a “clinical sample” which is a sample derived from a patient. The biological sample may contain any biological material suitable for detecting the desired biomarkers, and may comprise cellular and/or non-cellular material obtained from the individual. A biological sample can be obtained by appropriate methods, such as, by way of examples, blood draw, fluid draw, biopsy, or surgical resection. Examples of biological samples include but are not limited to blood, lymph, urine, saliva, mucus, plasma, biological tissue, feces, gastrointestinal fluid, semen, and biopsies. Samples that are liquid in nature are referred to herein as “bodily fluids”. Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample”, i.e., a sample derived from a patient. Such samples include, but are not limited to, bodily fluids which may or may not contain cells, e.g., blood (e.g., whole blood, serum or plasma), urine, saliva, tissue or fine needle biopsy samples, tissue sample obtained during surgical resection, and archival samples with known diagnosis, treatment, and/or outcome history. In some embodiments, the biological sample comprises a biological tissue of the subject, a blood sample of the subject, a bodily fluid sample of the subject, a fecal sample of the subject, a plasma sample of the subject, a saliva sample of the subject, a urine sample of the subject, or any combination thereof.

In one embodiment, the method comprises analyzing the biological sample with an assay that specifically detects a biomarker. In one embodiment, the method comprises analyzing the biological sample with an assay that specifically detects at least one biomarker. Examples of such assay include, but are not limited to: mass spectrometry (MS), liquid chromatography (LC), liquid chromatography-mass spectrometry (LC-MS), targeted liquid chromatography-tandem mass spectrometry (LC-MS/MS), high-performance liquid chromatography (HPLC), ultra-performance liquid chromatography (UPLC), ultra-high-performance liquid chromatography (UHPLC), gas chromatography (GC), gas chromatography-mass spectrometry (GC-MS), globally optimized targeted mass spectrometry, targeted assay of about 200 metabolites, aqueous global profiling, liquid global profiling, GC-MS profiling, GC-MS flux analysis, carnitine analysis, lipid targeted analysis, quantitative lipid targeted analysis, tryptophan analysis, absolute quantification, multivariate statistical analysis, dynamic light scattering (DLS), nuclear magnetic resonance (NMR) spectroscopy, ultraviolet-visible (UV/Vis) spectroscopy, infrared (IR) spectroscopy, Raman spectroscopy, or any combination thereof.

In one embodiment, the method comprises using a multi-dimensional non-linear algorithm to determine if the level (e.g., activity, amount, concentration, concentration of the ionized form, concentration of the neutral form, expression, level, etc.) of a set of biomarkers in the biological sample is statistically different than a comparator. In some embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

In one embodiment, the method comprises detecting one or more biomarkers in a biological sample of the subject. In some embodiments, the level of one or more of markers of the invention in the biological test sample of the subject is compared to a comparator. Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In one embodiment, the comparator is a level (e.g., activity, amount, concentration, concentration of the ionized form, concentration of the neutral form, expression, level, etc.) of the one or more biomarker in a sample obtained from a subject not having a disease or disorder associated with vitamin D deficiency or vitamin D imbalance. In one embodiment, the comparator is a level of the one or more biomarker in a sample obtained from a subject known not to have a disease or disorder associated with vitamin D deficiency or vitamin D imbalance.

In one embodiment, the comparator is a level of the one or more biomarker in a sample obtained from a subject having a different disease or disorder associated with vitamin D deficiency or vitamin D imbalance (e.g., cancer vs autoimmune disease or disorder vs diabetes vs adenoma vs metabolic syndrome vs chronic pain vs decline in renal function). In one embodiment, the comparator is a level of the one or more biomarker in a sample obtained from a subject known to have a different disease or disorder associated with vitamin D deficiency or vitamin D imbalance (e.g., cancer vs autoimmune disease or disorder vs diabetes vs adenoma vs metabolic syndrome vs chronic pain vs decline in renal function).

In one aspect, the present invention includes methods for identifying subjects who have vitamin D deficiency or vitamin D imbalance and subjects who do not have vitamin D deficiency or vitamin D imbalance by detection of the biomarkers disclosed herein. In some embodiments, the biomarkers are used to generate a biomarker profile or signature of the subjects: (i) who have vitamin D deficiency or vitamin D imbalance, and/or (ii) who do not have vitamin D deficiency or vitamin D imbalance.

In one aspect, the present invention includes methods for identifying subjects who are at risk of developing vitamin D deficiency or vitamin D imbalance and subjects who are not at risk of developing vitamin D deficiency or vitamin D imbalance by detection of the biomarkers disclosed herein. In some embodiments, the biomarkers are used to generate a biomarker profile or signature of the subjects: (i) who are at risk of developing vitamin D deficiency or vitamin D imbalance, and/or (ii) who are not at risk of developing vitamin D deficiency or vitamin D imbalance.

In one aspect, the present invention includes methods for identifying subjects who have a disease or disorder associated with vitamin D deficiency or vitamin D imbalance and subjects who do not have a disease or disorder associated with vitamin D deficiency or vitamin D imbalance by detection of the biomarkers disclosed herein. In some embodiments, the biomarkers are used to generate a biomarker profile or signature of the subjects: (i) who have a disease or disorder associated with vitamin D deficiency or vitamin D imbalance, and/or (ii) who do not have a disease or disorder associated with vitamin D deficiency or vitamin D imbalance.

In one aspect, the present invention includes methods for identifying subjects who are at risk of developing a disease or disorder associated with vitamin D deficiency or vitamin D imbalance and subjects who are not at risk of developing a disease or disorder associated with vitamin D deficiency or vitamin D imbalance by detection of the biomarkers disclosed herein. In some embodiments, the biomarkers are used to generate a biomarker profile or signature of the subjects: (i) who are at risk of developing a disease or disorder associated with vitamin D deficiency or vitamin D imbalance, and/or (ii) who are not at risk of developing a disease or disorder associated with vitamin D deficiency or vitamin D imbalance.

In one embodiment, the biomarker profile of a subject is compared to a predetermined or comparator biomarker profile or reference biomarker profile to identify vitamin D deficiency or vitamin D imbalance. In one embodiment, the biomarker profile of a subject is compared to a predetermined or comparator biomarker profile or reference biomarker profile to identify a risk of developing vitamin D deficiency or vitamin D imbalance. In one embodiment, the biomarker profile of a subject is compared to a predetermined or comparator biomarker profile or reference biomarker profile to identify a disease or disorder associated with vitamin D deficiency or vitamin D imbalance. In one embodiment, the biomarker profile of a subject is compared to a predetermined or comparator biomarker profile or reference biomarker profile to identify a risk of developing a disease or disorder associated with vitamin D deficiency or vitamin D imbalance. In one embodiment, the biomarker profile of a subject is compared to a predetermined or comparator biomarker profile or reference biomarker profile to diagnose a disease or disorder associated with vitamin D deficiency or vitamin D imbalance. In one embodiment, the biomarker profile of a subject is compared to a predetermined or comparator biomarker profile or reference biomarker profile to assess the prognosis of a disease or disorder associated with vitamin D deficiency or vitamin D imbalance. In one embodiment, the biomarker profile of a subject is compared to a predetermined or comparator biomarker profile or reference biomarker profile to evaluate the treatment of a disease or disorder associated with vitamin D deficiency or vitamin D imbalance. In one embodiment, the biomarker profile of a subject is compared to a predetermined or comparator biomarker profile or reference biomarker profile to distinguish between different types of diseases or disorders associated with vitamin D deficiency or vitamin D imbalance.

Control group samples may either be from a normal subject, samples from subjects with a known diagnosis of vitamin D deficiency or vitamin D imbalance or a disease or disorder associated with vitamin D deficiency or vitamin D imbalance, or samples from subjects with no known diagnosis of vitamin D deficiency or vitamin D imbalance or a disease or disorder associated with vitamin D deficiency or vitamin D imbalance. As described below, comparison of the expression patterns of the sample to be tested with comparators can be used to identify or diagnose vitamin D deficiency or vitamin D imbalance and/or a disease or disorder associated with vitamin D deficiency or vitamin D imbalance in the subject. In some instances, the control groups are only for the purposes of establishing initial cutoffs or thresholds for the assays of the invention. Therefore, in some instances, the systems and methods of the invention can identify or diagnose vitamin D deficiency or vitamin D imbalance and/or a disease or disorder associated with vitamin D deficiency or vitamin D imbalance without the need to compare with a control group.

Information obtained from the methods of the invention described herein can be used alone, or in combination with other information (e.g., age, family history, disease status, disease history, vital signs, blood chemistry, PSA level, Gleason score, lymph node staging, metastasis staging, expression of other gene signatures relevant to outcomes of a disease or disorder associated with vitamin D deficiency or vitamin D imbalance, etc.) from the subject or from the biological sample obtained from the subject. In some embodiments, the biomarkers data is combined or correlated with other data or test results that include, but are not limited to measurements or results from serologic testing methods, enzyme immunoassay (EIA), complement fixation (CF), immunodiffusion, clinical presentation, serology, radiography, histology, culture, and clinical parameters or other algorithms for developing or having vitamin D deficiency or vitamin D imbalance and/or a disease or disorder associated with vitamin D deficiency or vitamin D imbalance. In one embodiment, data include, but are not limited to age, ethnicity, PSA level, Gleason score, lymph node staging, metastasis staging, and other genomic data, and specific expression values of other gene signatures relevant to vitamin D outcomes. In one embodiment, the data comprises subject information, such as medical history, travel history, and/or any relevant family history. Several serology techniques that can be used in combination with the compositions and methods of the present invention. Examples of serology techniques include, but are not limited to: ELISA, agglutination, precipitation, complement-fixation, fluorescent antibodies, and chemiluminescence.

In certain embodiments, the method comprises using surgical data in combination with the detection of the relevant biomarkers described herein to diagnose, assess the prognosis, or assess the effectiveness of a treatment of vitamin D deficiency or vitamin D imbalance and/or a disease or disorder associated with vitamin D deficiency or vitamin D imbalance. For example, in certain embodiments, the method comprises assessing the severity of a disease or disorder associated with vitamin D deficiency or vitamin D imbalance, the spread of a disease or disorder associated with vitamin D deficiency or vitamin D imbalance to the lymph node (N category), or spread of a disease or disorder associated with vitamin D deficiency or vitamin D imbalance to other parts of the body (metastatic stage) (M category).

In one aspect, the invention contemplates the detection of differentially expressed biomarkers using tissue microarray. In one embodiment, the method comprises diagnosing vitamin D deficiency or vitamin D imbalance and/or a disease or disorder associated with vitamin D deficiency or vitamin D imbalance by detecting differentially expressed biomarkers in biological tissue excised from the subject during biopsy. In one aspect, the invention further contemplates using methods known to those skilled in the art to detect and to measure the level of one or more differentially expressed marker expression products.

In one embodiment, a cellular or fluid examination is used to detect or measure a variety of molecules including RNA, protein, and a number of molecules that are modified as a result of the protein's function. Exemplary diagnostic methods focusing on nucleic acids include but are not limited to amplification techniques, such as PCR and RT-PCR (including quantitative variants), and hybridization techniques, such as in situ hybridization, microarrays, and blots. Exemplary diagnostic methods focusing on proteins include but are not limited to binding techniques, such as ELISA, immunohistochemistry, microarray, and functional techniques, such as enzymatic assays.

The genes identified as being differentially expressed may be assessed in a variety of nucleic acid detection assays to detect or quantify the expression level of a gene or multiple genes in a given sample. For example, traditional Northern blotting, nuclease protection, RT-PCR, microarray, and differential display methods may be used for detecting gene expression levels. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays are most efficiently designed with array or chip hybridization-based methods for detecting the expression of a large number of genes. Any hybridization assay format may be used, including solution-based and solid support-based assay formats.

The protein products of the genes identified herein can also be assayed to determine the amount of expression. Methods for assaying for a protein include Western blot, immunoprecipitation, and radioimmunoassay. The proteins analyzed may be localized intracellularly (most commonly an application of immunohistochemistry) or extracellularly (most commonly an application of immunoassays such as ELISA).

In some embodiments, the invention relates to different types of biomarkers (e.g., protein level or activity, nucleic acid level or activity, mRNA level or activity, gene expression. etc.) and their measurements that can be combined with the compositions and methods of the present invention. In various embodiments, the neutral form of the biomarkers is measured. In various embodiments, the derivative form of the biomarkers is measured. In various embodiments, the ionized form of the biomarkers is measured. In various embodiments, measurements of neutral biomarkers are used in conjunction with measurements of ionized biomarkers. Biomarkers generally can be measured and detected through a variety of assays, methods and detection systems known to one of skill in the art. Various methods include but are not limited to immunoassays, microarray, PCR, RT-PCR, refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance. In this regard, biomarkers can be measured using the above-mentioned detection methods, or other methods known to the skilled artisan. Other biomarkers can be similarly detected using reagents that are specifically designed or tailored to detect them.

In some embodiments of the invention, methods of measuring biomarker levels in a biological sample obtained from a subject include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (MA), a radioimmunodiffusion assay, a liquid chromatography-tandem mass spectrometry assay, an Ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay.

The concentration of the biomarker in a sample may be determined by any suitable assay. A suitable assay may include one or more of the following methods, an enzyme assay, an immunoassay, mass spectrometry, chromatography, electrophoresis or an antibody microarray, or any combination thereof. Thus, as would be understood by one skilled in the art, the systems and methods of the invention may include any method known in the art to detect a biomarker in a sample.

The invention described herein also relates to methods for a multiplex analysis platform. In one embodiment, the method comprises an analytical method for multiplexing analytical measurements of markers.

Methods of Treatment

In some embodiments, the present invention further provides methods relating to the biomarkers of the invention that can be used to establish and evaluate treatment plans for a subject with vitamin D deficiency or vitamin D imbalance and/or a disease or disorder associated with vitamin D deficiency or vitamin D imbalance. In some embodiments, the invention includes methods for assessing the effectiveness of a treatment of vitamin D deficiency or vitamin D imbalance and/or a disease or disorder associated with vitamin D deficiency or vitamin D imbalance by detecting differentially expressed biomarkers in a biological sample obtained from a subject. For example, in certain embodiments, the method comprises assessing the effectiveness of a treatment of vitamin D deficiency or vitamin D imbalance and/or a disease or disorder associated with vitamin D deficiency or vitamin D imbalance by detecting differentially expressed biomarkers in a biological sample obtained from a subject having vitamin D deficiency or vitamin D imbalance and/or a disease or disorder associated with vitamin D deficiency or vitamin D imbalance or being treated for vitamin D deficiency or vitamin D imbalance and/or a disease or disorder associated with vitamin D deficiency or vitamin D imbalance.

In some embodiments of the invention, the methods comprise a) providing a biological sample from the subject; b) analyzing the biological sample with an assay that specifically detects at least one biomarker of the invention in the biological sample; c) comparing the level of the at least one biomarker in the sample to a comparator, wherein a statistically significant difference between the level of the at least one biomarker in the sample to a comparator or the level of the at least one biomarker in an earlier obtained biological sample is indicative of vitamin D deficiency or vitamin D imbalance and/or a disease or disorder associated with vitamin D deficiency or vitamin D imbalance in the subject. In some embodiments, the methods further comprise the step of d) effectuating a treatment regimen based thereon.

In some embodiments, the method of treatment comprises the detection of a differential expression of one or more biomarkers that indicate a treatment of the subject is needed. In one embodiment, the treatment is determined based on the level of the at least one biomarker in a subject. In some embodiments, the method of treatment includes, but is not limited to pharmacotherapy, surgery, radiation, and chemotherapy. In some embodiments, the method of treatment comprises administering a therapeutically effective amount of a drug. Examples of such drugs include, but are not limited to: a nucleic acid, a peptide, a small molecule chemical compound, an siRNA, a ribozyme, an antisense nucleic acid, an aptamer, a peptidomimetic, an antibody, an antibody fragment, an antibiotic, antifungal medication, and a combination thereof.

In some embodiments, the method of treatment comprises monitoring the biomarker levels during the course of treatment of a disease or disorder. In some embodiments, the method of treatment comprises an assessment of the effectiveness of the treatment regimen for vitamin D deficiency or vitamin D imbalance and/or a disease or disorder associated with vitamin D deficiency or vitamin D imbalance by detecting one or more biomarkers in an effective amount from samples obtained from a subject over time and comparing the amount of biomarker or biomarkers detected. In some embodiments, a first sample is obtained prior to the subject receiving treatment and one or more subsequent samples are taken after or during treatment of the subject. In some embodiments, changes in biomarker levels over time provide an indication of effectiveness of the therapy.

In one aspect, the biomarkers are used to monitor subjects undergoing treatments and therapies for vitamin D deficiency or vitamin D imbalance and/or a disease or disorder associated with vitamin D deficiency or vitamin D imbalance, subjects who have had vitamin D deficiency or vitamin D imbalance and/or a disease or disorder associated with vitamin D deficiency or vitamin D imbalance, and subjects who are in remission of a previously diagnosed and treated vitamin D deficiency or vitamin D imbalance and/or disease or disorder associated with vitamin D deficiency or vitamin D imbalance. In one embodiment, the biomarkers are used to select or modify treatments in subjects having vitamin D deficiency or vitamin D imbalance and/or a disease or disorder associated with vitamin D deficiency or vitamin D imbalance, subjects who have had vitamin D deficiency or vitamin D imbalance and/or a disease or disorder associated with vitamin D deficiency or vitamin D imbalance, and subjects who are in remission of a previously diagnosed and treated vitamin D deficiency or vitamin D imbalance and/or disease or disorder associated with vitamin D deficiency or vitamin D imbalance.

In some embodiments, the methods of the present invention comprise effecting a therapy and/or the treatment regime based on the diagnosis or assessment of prognosis of vitamin D deficiency or vitamin D imbalance and/or a disease or disorder associated with vitamin D deficiency or vitamin D imbalance. In one embodiment, the treatment is adjusted based on the level of the at least one biomarker in a subject.

In one aspect, the present invention also provides methods for identifying agents for treating vitamin D deficiency or vitamin D imbalance and/or a disease or disorder associated with vitamin D deficiency or vitamin D imbalance that are appropriate or otherwise customized for a specific subject. In one embodiment, a test sample from a subject, exposed to a therapeutic agent or a drug, can be taken and the level of one or more biomarkers can be determined. In one embodiment, the level of one or more biomarkers can be compared to a sample derived from the subject before and after treatment, or can be compared to samples derived from one or more subjects who have shown improvements or alleviation of vitamin D deficiency or vitamin D imbalance and/or a disease or disorder associated with vitamin D deficiency or vitamin D imbalance as a result of such treatment or exposure.

To identify therapeutics or drugs that are appropriate for a specific subject, a test sample from the subject can also be exposed to a therapeutic agent or a drug, and the level of one or more biomarkers can be determined. Biomarker levels can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising a step of taking a first measurement of a biomarker panel in a first sample from the subject; a step of effecting the therapy with respect to the subject; a step of taking a second measurement of the biomarker panel in a second sample from the subject; and a step of comparing the first and second measurements to assess the efficacy of the therapy.

Additionally, therapeutic agents suitable for administration to a particular subject can be identified by detecting one or more biomarkers in an effective amount from a sample obtained from a subject and exposing the subject-derived sample to a test that determines the amount of the biomarker(s) in the subject-derived sample. Accordingly, treatments or therapeutic regimens for use in subjects having an enhanced risk for developing a disease or disorder associated with vitamin D deficiency or vitamin D imbalance can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

In various exemplary embodiments, the methods of the invention include effecting a therapy for the treatment of a diagnosed disease. In one embodiment, effecting a therapy comprises administering a disease-modulating drug to the subject. The subject may be treated with one or more drugs until altered levels of the measured biomarkers return closer to the baseline value measured in a population not having vitamin D deficiency or vitamin D imbalance and/or a disease or disorder associated with vitamin D deficiency or vitamin D imbalance, not having recurrence of vitamin D deficiency or vitamin D imbalance and/or a disease or disorder associated with vitamin D deficiency or vitamin D imbalance, or showing improvements in disease biomarkers as a result of treatment with a drug. Additionally, improvements related to a changed level of a biomarker or clinical parameter may be the result of treatment with a disease-modulating drug. For example, in one embodiment, effecting a therapy comprises administering an antifungal drug to a subject. Exemplary antifungal agents that can be administered include, but are not limited to, fluconazole and itraconazole.

In various embodiments, effecting a therapy comprises administering a therapeutically effective amount of one or more agonists or antagonists of calcitriol, one or more agonists or antagonists of 24,25-dihydroxyvitamin D, one or more agonists or antagonists of calcifediol, one or more agonists or antagonists of vitamin D metabolizing enzymes (e.g., CYP27B1, CYP2R1, CYP3A family, CYP24A1), or any combination thereof. For example, in some embodiments, the treatment comprises administering a therapeutically effective amount of one or more inhibitors of calcitriol, one or more inhibitors of 24,25-dihydroxyvitamin D, one or more inhibitors of calcifediol, or any combination thereof. In some embodiments, the prevention comprises administering a therapeutically effective amount of one or more inhibitors of calcitriol, one or more inhibitors of 24,25-dihydroxyvitamin D, one or more inhibitors of calcifediol, or any combination thereof.

In various embodiments, effecting a therapy comprises treatment of one or more symptoms of the disease or disorder. For example, in one embodiment, effecting a therapy comprises administration of a non-disease-modulating drug to the subject. Exemplary non-disease-modulating drugs that may be administered include, but are not limited to, pain relievers, anti-inflammatory drugs, NSAIDs, decongestants, cough suppressants, including topical cough suppressants, or other agents that may function to reduce the severity of at least one symptom of the disease or disorder.

Any drug or any combination of drugs disclosed herein may be administered to a subject to treat a disease. The drugs herein can be formulated in any number of ways, often according to various known formulations in the art or as disclosed or referenced herein.

In various embodiments, any drug or any combination of drugs disclosed herein is not administered to a subject to treat a disease. In these embodiments, the practitioner may refrain from administering the drug or any combination of drugs, may recommend that the subject not be administered the drug or any combination of drugs or may prevent the subject from being administered the drug or any combination of drugs.

In various embodiments, one or more additional drugs may be optionally administered in addition to those that are recommended or have been administered. An additional drug will typically not be any drug that is not recommended or that should be avoided.

### Kits

The present invention also pertains to kits useful in the methods of the invention. Such kits comprise various combinations of components useful in any of the methods described elsewhere herein, including for example, materials for quantitatively analyzing a biomarker of the invention, materials for assessing the activity of a biomarker of the invention, and instructional material. For example, in one embodiment, the kit comprises components useful for the quantification of a desired biomarker in a biological sample. In a further embodiment, the kit comprises components useful for the assessment of the activity of a desired biomarker in a biological sample.

In a further embodiment, the kit comprises the components of an assay for monitoring the effectiveness of a treatment administered to a subject in need thereof, containing instructional material and the components for determining whether the level of a biomarker of the invention in a biological sample obtained from the subject is modulated during or after administration of the treatment. In various embodiments, to determine whether the level of a biomarker of the invention is modulated in a biological sample obtained from the subject, the level of the biomarker is compared to at least one comparator contained in the kit, such as a positive control, a negative control, a historical control, a historical norm, or the level of another reference molecule in the biological sample. In certain embodiments, the ratio of the biomarker and a reference molecule is determined to aid in the monitoring of the treatment.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Ancestry Adjusted Calcitriol-24,25-Dihydroxyvitamin D3-Calcifediol Proportion Ratio (C24CPR) for Secondary and Tertiary Prevention

The largest randomized trial of high dose vitamin D treatment (cholecalciferol, 2000 IU/d) has reported no benefit in prevention of the incidence of cancer, heart disease and stroke, although a significant benefit in the reduction of cancer mortality was observed—particularly among individuals with lower body mass index (Chandler P D et al., 2020, JAMA Netw Open. 3(11):e2025850; Manson J E et al., 2020, J Steroid Biochem Mol Biol, 198:105522). In addition, the increasing population prevalence of chronic kidney disease which is now ˜15% in the US population (US Centers for Disease Control and Prevention, 2021, cdc.gov/kidneydisease/publications-resources/ckd-national-facts.html), which is associated with low circulating calcitriol concentrations (Selamet U, et al., 2018, Am J Kidney Dis, 72(3): 419-428., indicated that innovative non-calcemic analogs of calcitriol (i.e., those which do not require renal hydroxylation) to prevent vitamin D deficiency will become more important.

CYP24A1 is the major vitamin D catabolic enzyme for which at least three major metabolites compete. These include 25(OH)D₃ (i.e., calcifediol, calcidiol, 25-hydroxycholecalciferol, 25-hydroxyvitamin D3), 24,25(OH)₂D₃ (i.e., 24,25-dihydroxycholecalciferol, also known as 24,25-dihydroxyvitamin D3 and-hydroxycalcidiol), and the active form of vitamin D (i.e., calcitriol, 1,25-dihydroxycholecalciferol, 1,25(OH)₂D₃). The calcitriol (i.e., 1,25(OH)₂D₃) to 24,25-dihydroxyvitamin D3 (i.e., 24,25(OH)₂D₃) to calcifediol (i.e., 25(OH)D₃) proportion ratio (C24CPR) biomarker was developed under the concept of CYP24A1 enzyme metabolite competition.

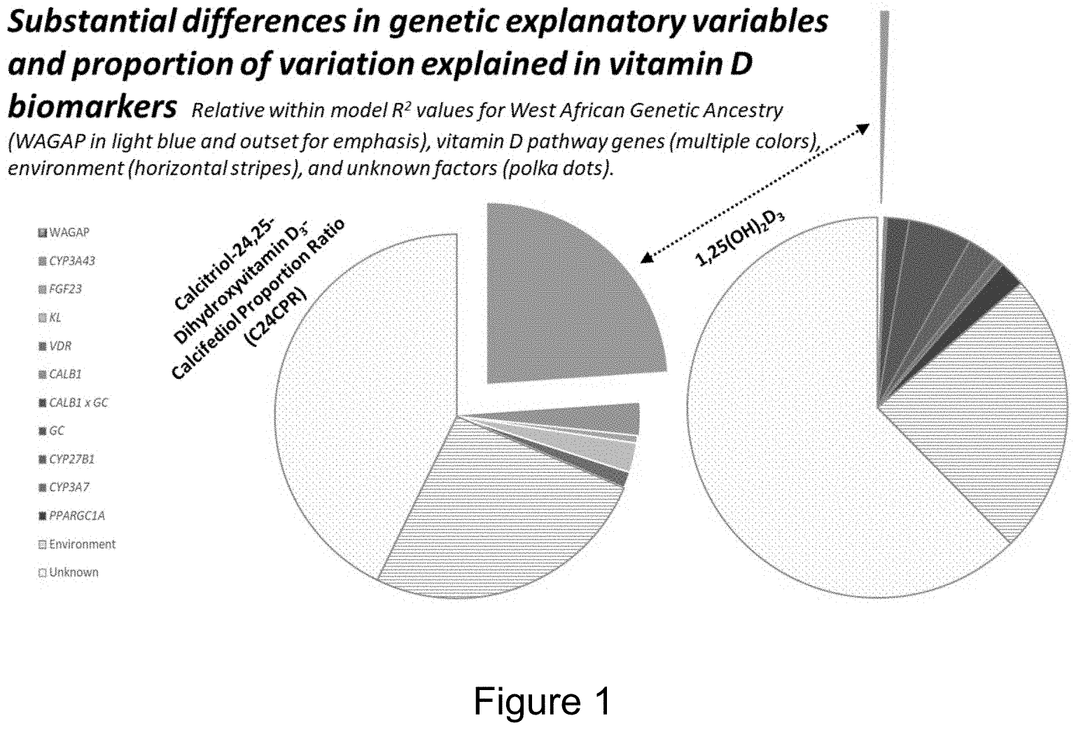

All three metabolites, which make up the balance of C24CPR, compete for catabolism by this mitochondrial enzyme. The “active” form of vitamin D, known as calcitriol (i.e., 1,25(OH)₂D₃) is the only vitamin D metabolite known to influence downstream gene function via the vitamin D receptor (VDR) at picomolar concentrations. As discussed herein, higher C24CPR values were shown to describe a greater production of 1,25(OH)₂D₃ relative to 24,25(OH)₂D₃, adjusted for the upstream reserve of 25(OH)D₃. C24CPR calculation required plasma measurement of all three metabolites using high performance liquid chromatography and tandem mass spectrometry (HPLC-MS/MS). Details regarding the assay and development of ancestry informative markers were provided in Wilson 2018 (Wilson et al., 2018, Am J Epidemiol., 187:754-766) and Wilson 2011 (Wilson et al., 2011, Horm Mol Biol Clin Investig., 7:279-293); details regarding the calculation were provided in Example 2 below; and molecular rationale for differential CYP24A1 expression according to genetic ancestry (Roff et al., 2008, J Steroid Biochem Mol Biol., 112:47-54). Ancestry-adjustment of C24CPR required genotyping for Ancestry Informative Markers (ATMs) (Wilson et al., 2018, Am J Epidemiol., 187:754-766 and Example 2 below) and, in some cases, a correlation between genetic variability (i.e., frequency of novel single nucleotide polymorphic variants) was observed among African populations (McEvoy et al., 2011, Genome Res., 21:821-829). This was a major reason why a West African genetic-ancestry-adjusted measure of C24CPR was developed herein (FIG. 1).

The present studies have developed this biomarker among healthy young adults and assessed its characteristics in association with respect to dietary intake, sunlight exposure, genetic ancestry and several other vitamin D pathway gene variants. Two limitations were: approximately 30% of variability in C24CPR was associated with the proportion of West African genetic ancestry in the studied sample of African American and European American participants. This provided a major rationale for the development of vitamin D measures adjusted for African genetic ancestry. Therefore an ancestry-adjusted measure can be used. The limitation of using an ancestry-adjusted measure was the need to genotyping. Genotyping for ancestry determination is presently being used in direct to consumer marketing (e.g. 23andMe), however, it is not known how accurate these methods are. Second limitation related to a lack of assessment of this measure in other populations. Thus, additional studies are focusing on assessing longer term variability in other populations, including those with renal disease, cancer, children, and older adults.

The US Preventive Services Task Force (USPSTF) has concluded that there is ‘adequate evidence not to recommend’ use of the 25(OH)D₃ blood biomarker as a screening tool for the treatment of asymptomatic vitamin D deficiency or vitamin D imbalance—particularly for the prevention of cancer, type 2 diabetes mellitus, total mortality, and/or fracture risk (USPSTF, 2018, JAMA, 319:1592-159; Kahwati L C, et al. Screening for Vitamin D Deficiency in Adults: An Evidence Review for the U.S. Preventive Services Task Force [Internet]. Rockville (Md.): Agency for Healthcare Research and Quality (US); 2021 April (Evidence Synthesis, No. 201.) ncbi.nlm.nih.gov/books/NBK569723/). Vitamin D supplementation clearly resulted in increases in 25(OH)D (either 25(OH)D₂ or 25(OH)D₃ depending on the supplement form), due to constant production by CYP2R1 enzyme. In spite of a nearly ˜40% increase in 25(OH)D₃ among healthy individuals taking 2000 IU/day of vitamin D in the VITAL Study (the largest clinical trial of higher dose vitamin D supplementation ever conducted), there was no benefit for reducing the incidence of cancer, cardiovascular disease, or stroke in older adults, when compared with placebo after 5 years (Manson, 2019, N Engl J Med, 380:33-44). However, a significant benefit in the reduction of cancer mortality was observed—particularly among individuals with lower body mass index (Chandler P D et al., 2020, JAMA Netw Open. 3(11):e2025850; Manson J E et al., 2020, J Steroid Biochem Mol Biol, 198:105522). Vitamin D (400 IU/day) plus calcium supplementation was also not associated with cancer risk in longer term follow-up of the Women's Health Initiative and was associated with an increased risk for renal stones (Jackson, 2006, N Engl J Med, 354:669-683).

In spite of this, cord blood 25(OH)D concentration has been associated with later elevations in systolic blood pressure in adults in multiple long-term cohort studies (Sauder K A, 2019, J Am Heart Assoc, 8(9):e011485), indicating that very low cord blood levels at birth (<11 ng/mL) are a concern Thus, there may be applicability of vitamin D biomarkers in prediction of hypertensionin early life (Sauder K A, 2019, J Am Heart Assoc, 8(9):e011485,) and young adulthood (Cuffee et al., 2021, J Hum Hypertens, doi: 10.1038/s41371-021-00577-6). These studies indicated that the 25(OH)D biomarker of the available pool of vitamin D may be important. The 25(OH)D biomarker measures an available nanomolar pool of circulating vitamin D but does not describe its balance with respect to other metabolites—particularly the “active form” of vitamin D (1,25(OH)₂D).

Two metabolites immediately downstream from 25(OH)D include calcitriol (i.e., 1,25(OH)₂D₃) and 24,25(OH)₂D₃. Calcitriol (i.e., 1,25(OH)₂D₃), produced by CYP27B1 and catabolized by CYP24A1, circulates at a concentration approximately 1000-fold lower (picomolar concentrations) than 25(OH)D (nanomolar concentrations), and is presently under investigation by USPSTF (USPSTF, 2020, uspreventiveservicestaskforce.org/uspstf/draft-update-summary/vitamin-d-deficiency-screening1).

Calcitriol (i.e., 1,25(OH)₂D₃) is used clinically to aid the diagnosis of primary hyperparathyroidism, hypoparathyroidism, pseudohypoparathyroidism, renal osteodystrophy (in the setting of chronic kidney disease), and vitamin D-resistant rickets. Calcitriol (i.e., 1,25(OH)₂D₃) is not presently used for screening asymptomatic adults, most likely due to past problems with interference in the 1,25(OH)₂D₃ radioimmunoassay (MA), diurnal variation in circulation, and known influence of estrogens which may increase values up to 30% (e.g., oral contraceptives) compared with non-users.

Furthermore, the 24,25(OH)₂D₃ metabolite is produced by CYP24A1 enzyme. The ratio of 24,25(OH)₂D₃ to 25(OH)D is used to monitor vitamin D therapy in chronic kidney disease (Mayo Clinic) and is thought to have influence on bone resorption and remodeling.

Uses of C24CPR

Exemplary Use 1: Screening Among Healthy Individuals

Calcitriol (1,25(OH)₂D₃) and 25(OH)₂D₃ alone has been associated with a healthy lifestyle (Kohler, 2017, J Nutr, 147:421-429). Healthy individuals randomized to different doses of vitamin D supplementation have been shown to increase 24,25(OH)₂D₃ to a greater extent than calcitriol (1,25(OH)₂D₃)(Owens, 2016, Med Sci Sports Exerc, 49:349-356). In the present studies, the crude ratio of 1,25(OH)₂D₃ was used to monitor this effect. The present studies have shown that, compared with a crude ratio, improved distributional characteristics of C24CPR (less skewness, lower dispersion with greater proportion of overall variability explained by well-known genetic and environmental factors) may facilitate its applicability to population interventions. This means that C24CPR is more conducive to the development of genetically-defined cut-off values, as well as strengthening statistical power for testing for statistical differences in populations. Thus, C24CPR “reduced noise” as a biomarker of vitamin D status by adjusting for the upstream pool of 25(OH)D₃.

Expression of the enzymes that metabolize calcitriol (e.g., CYP27B1) and metabolize 24,25(OH)₂D₃ (e.g., CYP24A1) are most highly expressed in renal tissues (Human Protein Atlas, 2020, proteinatlas.org/ENSG00000019186-CYP24A1/tissue and proteinatlas.org/ENSG00000111012-CYP27B1/tissue). Calcitriol (1,25(OH)₂D₃) decline was strongly correlated with advancing renal disease](Bosworth C, 2013 Semin Nephrol, 33(2):158-68). In addition, “stagnation of 24,25(OH)₂D₃ metabolism” is a hallmark of chronic kidney disease (Bosworth, 2013, Semin Nephrol, 33:158-168). In randomized trials, vitamin D treatment is associated with lower total mortality and cardiovascular mortality in patients with chronic kidney disease and patients with end stage renal disease (ESRD) (Zheng Z et al., 2013, BMC Nephrol, 14:199. In addition, the increasing population prevalence of chronic kidney disease which is now ˜15% in the US population (US Centers for Disease Control and Prevention, 2021, cdc.gov/kidneydisease/publications-resources/ckd-national-facts.html), suggests there is a growing need for early screening. Taken together, this research highlighted the need to assess C24CPR in healthy individuals to identify decrements in renal function.

C24CPR is a distinct marker of vitamin D status in healthy young adults. Thus, the present studies demonstrated that the pattern of gene variants associated with the C24CPR biomarker is not the same as the pattern of gene variants associated with calcitriol (1,25(OH)₂D₃). Furthermore, sunlight and tanning bed use are well-known to increase plasma/serum calcitriol (1,25(OH)₂D₃) and 25(OH)D₃ concentrations through upregulation of CYP27B1 and to increase the available circulation D3 for conversion by CYP2R1, respectively. In contrast, the present studies demonstrated a significant dose-response decrease in C24CPR in association with these two factors. This result was unexpected. UV-induced DNA damage and the role of 1,25(OH)₂D₃ in upregulating p53, VDR and other responses may shunt 1,25(OH)₂D₃ from circulation to skin cells, resulting in a relative decrease of 1,25(OH)₂D₃ when compared with 24,25(OH)₂D₃. Oral contraceptive use is also well-known to increase plasma/serum calcitriol (1,25(OH)₂D₃) and 25(OH)D concentrations. The present studies demonstrated a significant decrease in C24CPR with among oral contraceptive users that appeared to be dose-dependent. Lastly, compared with calcitriol (1,25 (OH)₂D₃), a greater proportion of the C24CPR biomarker can be explained by known genetic and environmental factors (49.5% versus 37.4%, respectively).

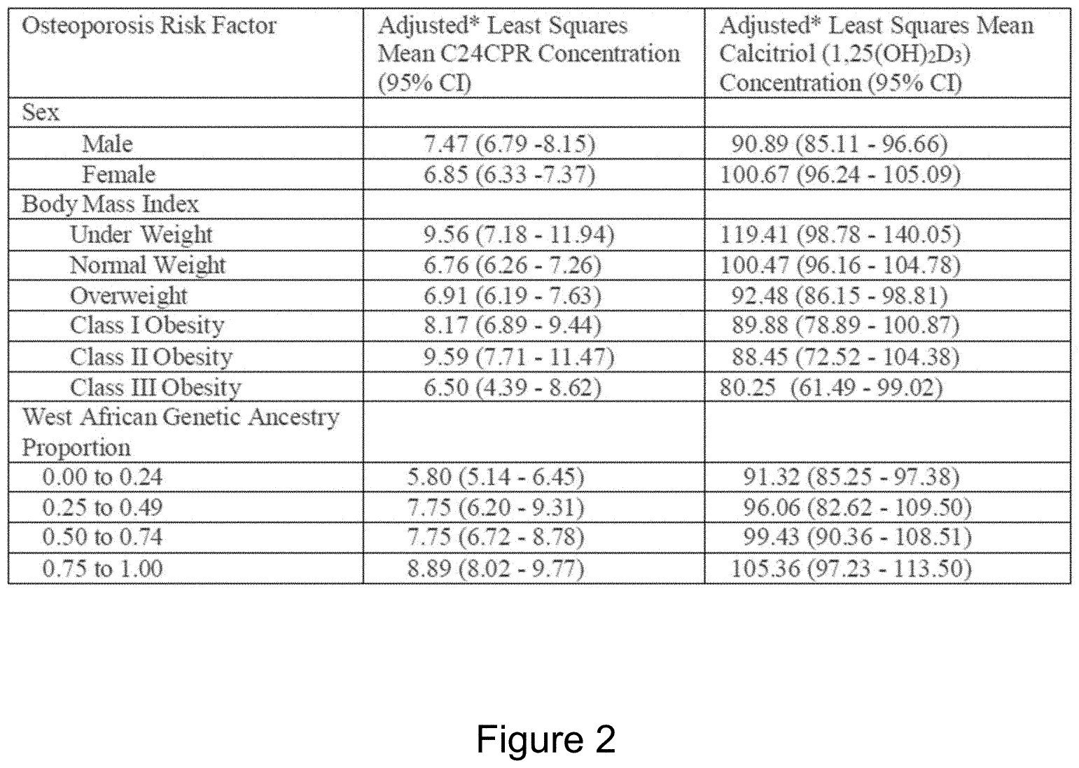

Factors associated with higher bone mineral density (i.e., male sex, higher BMI, and African American ancestry) were associated with higher C24CPR values in young healthy adults (ages 18-35). These associations were distinct from those with calcitriol, as shown in FIG. 2. For example, the association between BMI and C24CPR was not linear, whereas the association between BMI and calcitriol showed a linear decline.

Exemplary Use 2: Screening for CYP24A1 Inhibitor Therapy

Calcitriol has been shown to have strong anticancer (apoptotic) properties in multiple cell types in tissue culture. Treatment with calcitriol is hampered by its calcemic side effects. The CYP24A1 gene has been recently postulated to be an oncogene (i.e., gene expression is increased in several cancer types, ncbi.nlm.nih.gov/gene/1591). Promising CYP24A1 blockers have been developed as a way to slow catabolism of calcitriol (“the active form of vitamin D”) so that calcitriol's anti-cancer activity at the tissue level can be prolonged (Hartfield, 2012, J Steroid Biochem Mol Biol, 136:47-53). The contribution of CYP24A1 blockers avoid hypercalcemic side effects of direct dosing with calcitriol. Individuals on CYP24A1 inhibitors were expected to have higher C24CPR values.

Exemplary Use 3: Monitoring for Risk of Bone Depletion in Glucocorticoid Treatment

Glucocorticoid (e.g., dexamethasone) treatment in asthma and rheumatoid arthritis increases the risk for osteoporosis. Dexamethasone treatment in animal models resulted in increased CYP27B1 and CYP24A1 expression in multiple tissue types (Zhang, 2018, Hypertens Res, 41:104-111). The net result of enhancement of calcitriol production and catabolic enzymes was an overall decrease in serum calcitriol (1,25(OH)₂D₃), which was thought to be the major reason for osteoporosis development. Accordingly, the balance of calcitriol may also be important in this setting.

Exemplary Use 4: Screening for Idiopathic Infantile Hypercalcemia (IIH) and Reduced Function CYP24A1 Mutations

Loss of function germline mutations in CYP24A1 leading to IIH had a low prevalence in clinical populations (0.6%). There are also reduced function mutations in CYP24A1 that have been associated with renal stone development and may occur as frequently as 20%. In lieu of specific genetic testing, the C24CPR assay can be applied to screening for IIH and/or the risk for renal stone development at birth in order to promote clinical surveillance and prevent nephrolithiasis and subsequent renal damage.

In summary, the US Preventive Services Task Force and the National Academy of Medicine (formerly the Institute of Medicine) has called for improved circulating biomarkers of vitamin D status to be used in screening healthy adults for low vitamin D status. In light of this recognized need, the herein described studies focused on evaluation of the C24CPR.

Higher C24CPR values described a greater production of 1,25(OH)₂D₃ relative to 24,25(OH)₂D₃, adjusted for the upstream reserve of 25(OH)D₃. Adjustment using genetic ancestry informative markers (ATMs) further improve the use of C24CPR in precision medicine settings.

### Example 2: Evidence of C24CPR as a Distinct Biomarker of Vitamin D Status Comparison of Genetic Factors Associated with Circulating Concentrations of Calcitriol and a New Measure of Vitamin D Status, the Calcitriol-24,25-Dihydroxyvitamin D3-Calcifediol Proportion (C24CPR) Ratio

Two major enzymes control the balance of circulating 1,25(OH)₂D₃-CYP27B1 and CYP24A1. CYP27B1 is the exclusive mitochondrial 1,25(OH)₂D₃ production enzyme (St-Arnaud R et al., 1997, J Bone Miner Res, 12:1552-1559). Genetic variants near the CYP27B1 gene have been associated with multiple sclerosis (Ramagopalan S V et al., 2011, Ann Neurol, 70:881-886; Alcina A et al., 2013, J Med Genet, 50:25-33), type 1 diabetes (Lopez E R et al., 2004, Eur J Endocrinol, 151:193-197; Penna-Martinez Metal., 2017, Genes (Basel), 8:E125), autoimmune thyroiditis (Lopez E R et al., 2004, Eur J Endocrinol, 151:193-197), and differential CYP27B1 expression (Alcina A et al., 2013, J Med Genet, 50:25-33; Shahijanian F et al., 2014, Hum Mol Genet, 23:1425-1434).

Mitochondrial CYP24A1 is the chief vitamin D catabolic enzyme. CYP24A1 is most highly expressed in cell lines treated with 1,25(OH)₂D₃ and produces 24,25(OH)₂D₃, from 25(OH)D₃ (Pike J W et al., 2017, Endocrinol Metab Clin North Am, 46:815-843). Genetic variation in CYP24A1 has been associated with circulating concentrations of 24,25(OH)₂D₃ (Wilson R T et al., 2018, Am J Epidemiol, 187:754-766).) and differential CYP24A1 expression (Jacobs E T et al., 2013, Cancer Res, 73:2563-2573; Roff A et al., 2008, Journal of Steroid Biochemistry & Molecular Biology, 112:47-54). Aberrant CYP24A1 expression in several cancers has led to its classification as an oncogene (Horvath H C et al., 2010, J Histochem Cytochem, 58:277-285), possibly contributing to chemotherapy resistance (Zou M et al., 2017, Cancer Res, 77:2161-2172) and risk for cancers of the colon (Dong L M et al., 2009, Cancer Epidemiol Biomarkers Prev, 18:2540-2548) and breast (Reimers L L et al., 2015, Cancer Causes Control, 26:187-203). CYP24A1 is the major catabolic enzyme for three circulating vitamin D metabolites—including 25(OH)D₃ and both of its immediate downstream metabolites 1,25(OH)₂D₃ and 24,25(OH)₂D₃. It therefore seems plausible that a relative ratio of these CYP24A1-competing vitamin D metabolites may be more important than any single metabolite alone (Bosworth C et al., 2013, Semin Nephrol, 33:158-168; Tieu E W et al., 2014, FEBS J, 14:3280-3296). Understanding biomarker variability in healthy populations is important before implementation as a potential tool in pre-clinical and disease states.

Thus, the purpose of this study is to investigate major attributes of a possible alternative biomarker of vitamin D status, the calcitriol-24,25-dihydroxyvitamin D3-calcifediol proportion ratio (C24CPR). The distributional characteristics of C24CPR was compared to a crude ratio and genetic factors associated with C24CPR and calcitriol (1,25(OH)₂D₃) were compared and contrasted.

The materials and methods employed in the present experiments are now described herein.

Participants

Healthy individuals residing in Central Pennsylvania, ages 18 to 55 years, self-reporting at least 50% European (EA) or 50% African ancestry (AA), and providing written informed consent were enrolled (PSU IRB #43010). These two groups were chosen because of the substantial (˜2-fold) difference in serum 25(OH)D₃ between them (LeFevre M L et al., 2015, Ann Intern Med, 162:133-140). Individuals with fever (37.8° C. or higher), severe chronic disease (kidney disease, liver disease, chronic obstructive pulmonary disease, HIV/AIDS), unable to provide a blood sample, or currently undergoing chemotherapy or radiation treatment were excluded. At enrollment, EA participants were matched in a 1:1 ratio by age (+/−5 years) and sex with AA participants, as previously described (Wilson R T et al., 2018, Am J Epidemiol, 187:754-766).

Potential Confounding Variables and Sources of Vitamin D

Participants completed a questionnaire including demographic characteristics, personal medical history, current medication use, smoking status, dietary intake (including food and dietary supplements) and vitamin D exposure. Skin melanin index, height, weight, and resting (sitting) blood pressure (systolic and diastolic), and time of day were recorded at an in-person visit to the Clinical Research Center. All blood samples were collected during non-summer months in order to minimize seasonal variation (i.e., blood was not collected from May 15^{th} to August 31^{st}). At the visit, two measurements (averaged for the analysis) were taken each for blood pressure and LED reflectance-estimated skin melanin index of the upper inner arm (DermaSpectrometer II; CyberDerm, Media, Pa.). All current medications were reviewed by study staff and classified according to the Prescriber's Digital Reference (physician.pdr.net/drug-information/) as corticosteroid, hormone replacement use, oral contraceptive dose (mg/day), and statin use.

Sources of vitamin D included current tanning bed use, tanning bed hours per week (past month), hours spent outdoors in the past month, current outdoor occupation, and dietary intake. Usual dietary and supplement intake over the past year was estimated using a 24-item calcium and vitamin D screener based on the Block food frequency questionnaire including food frequency, as previously described (Wilson R T et al., 2018, Am J Epidemiol, 187:754-766). The screener included alcohol intake (wine, wine coolers, hard liquor, or mixed drinks) and coffee, including use of dairy and non-dairy creamers. Nutrients were calculated using information from the National Cancer Institute's 2009 Diet History Questionnaire Nutrient & Food Group Database made available with Diet*Calc software, a compilation of US Department of Agriculture's Food and Nutrient Database for Dietary Studies, and USDA's MyPyramid Equivalents Database (EGRP, epi.grants.cancer.gov/DHQ/database/current.html).

Biomarker Measurement

Four vitamin D metabolites (1,25(OH)₂D₃, 25(OH)D₂, 25(OH)D₃, and 24,25(OH)₂D₃) were measured in plasma (nmol/L) using high-performance liquid chromatography with tandem mass spectrometry (LC-MS/MS), as previously described (Wilson R T et al., 2011, Hormone Molecular Biology and Clinical Investigation, 7:279-293; Dai J P et al., chromatographyonline.com/sensitive-and-cost-effective-lc-ms-ms-method-determination-1-25-dihydroxyvitamin-d3-human-plasma-0). Plasma samples were prepared by solid phase extraction (SPE), spiked with internal deuterated standards (d6-1,25(OH)₂D₃, d6-25(OH)D₃, d6-25(OH)D₂, and d6-24,25(OH)₂D₃ (Chemaphor, Inc., Ottawa, Canada). Because calcitriol circulates at a much lower concentration, samples were concentrated using ImmunoTube extraction (Immundiagnostik AG, distributed by ALPCO) prior to SPE. The percent coefficient of variation (CV %), assayed in 10 repeated samples of pooled plasma from healthy individuals on separate days, was 2.7, 12.2, 6.3, and 7.2% for 25(OH)D₃, 25(OH)D₂, 24,25(OH)₂D₃, and 1,25(OH)₂D₃, respectively. In three consecutive batches, the calibration curves for 1,25(OH)₂D₃ showed an overall accuracy of 94.1-106.1% over the concentration range of 5.0-1000.0 pg/mL. The standard curve ranges for 25(OH)D₂ and 25(OH)D₃ was 1-100 ng/mL and for 24,25(OH)₂D₃ was 0.1-10 ng/mL. The lower limit of quantitation (LLOQ) was defined as the lowest calibration concentration with a signal-to-noise ratio of at least 5:1. The LLOQ for 1,25(OH)₂D₃ was 5.0 pg/mL (13 pmol/L), and for 25(OH)D₂, 25(OH)D₃ and 24,25(OH)₂D₃ metabolites the LLOQ was 10 pg/mL (˜25 pmol/L). Total serum calcium was measured in duplicate using colorimetry (BioVision, Mountain View, Calif.), CV %=3.5%.

Genotyping and Genetic Ancestry

Vitamin D-pathway genes included major vitamin D metabolizing enzymes (CYP2R1, CYP27B1, CYP24A1), lesser/suspected vitamin D metabolizing enzymes (CYP27A1, CYP3A4, CY3A43, CYP3A5, CYP3A7), inhibitors of CYP27B1 expression (FGF23, Klotho (KL), GALNT3), the extracellular calcium-sensing receptor (CASR), a cytosolic calcium binding protein (Calbindin d28K (CALB1)), calcium channels (TRPV5 and TRPV6)), the nuclear hormone receptor vitamin D receptor (VDR), a VDR co-activator (PPARGC1A), the vitamin D binding protein (GC) and megalin (LRP2), which is responsible for renal vitamin D uptake. Genes influencing skin pigmentation (SLC24A5, SLC45A and TMEM33) and familial conditions (i.e., Fanconi's Syndrome (SLC34A1) and familial hypophosphatemia (DMP1)) were also included. Single nucleotide polymorphic (SNP) variants within these genes were selected with preference for coding-region variants, those with a previously published association, and SNPs located near positive or negative vitamin D response elements (VDRE or nVDRE, respectively), or previously mechanistically-tested using either human (Jacobs E T et al., 2013, Cancer Res, 73:2563-2573; Maguire O et al., 2012, Mol Cell Endocrinol, 364:54-64; Turunen M M et al., 2007, Nucleic Acids Res, 35:2734-2747), or murine (Gill R K et al., 1993, Proc Natl Acad Sci USA, 90:2984-2988) DNA sequences. Promoter (p) and 3′untranslated region (UTR) variants were located according to their position upstream of the transcription start site or downstream of the 3′ UTR, respectively, using the human genome assembly 38 (hg38) sequence, dbSNP Build 150 and the following cataloged sequences in RefSeq for CALB1 (NM_004929), CYP24A1 (NM_000782), CYP3A4 (NM_017460), CYP3A7 (NM_000765), GALNT3 (NM_004482), and TMEM33 (NM_018126).

Because vitamin D metabolites have been strongly associated with West African genetic ancestry, a panel of 112 ancestry informative markers (AIMS) was used to estimate the proportion of West African Ancestry and proportion of European Ancestry, using the STRUCTURE algorithm (a Bayesian based cluster approach) and reference frequencies in HapMap YRI (West African) and CEU (European) trios, as previously described (Wilson et al., 2018, Am J Epidemiol., 187:754-766). This panel exhibits high agreement with other ancestry panels (concordance correlation coefficient=0.97) (Wilson R T et al., 2011, Hormone Molecular Biology and Clinical Investigation, 7:279-293).

DNA was extracted from whole blood in EDTA tubes using a QIAamp DNA Mini Kit (QIAGEN, USA) and stored at −80° C. The Illumina Bead Xpress was used to determine genotypes for AIMs and single nucleotide polymorphic variants (SNV), as listed in Table 1.

TABLE 1
Genes and single nucleotide variants investigated (promoter (p), amino acid changes
in the coded protein (capital letters), *allele reference, or untranslated region
(UTR)). All variants are genotyped with Illumina Bead Xpress. Underlined are genotyped
via TaqMan assay using RT-PCR due to failed genotyping in Illumina.

| | |
|---|---|
| 1 | CALB1 (rs2142093 (p⁻⁴¹⁰), rs2142094 (p⁻⁵⁰²), and rs3026284) |
| | CALB1 (rs1800645 (p⁻³⁶⁶) and rs16902897 (p⁻⁴⁹⁰)) |
| 2 | CASR (rs1801726 (E¹⁰¹¹Q) |
| 3 | CALCA (rs34414857, rs5241, rs34164367) |
| 4 | CYP2R1 (rs61495246 (L⁹⁹P) |
| | CYP2R1 (rs10741657 (p⁻¹¹²⁷), rs2060793 (p⁻¹⁵⁵⁹) and rs16930609 (p⁻²¹⁵⁷)) |
| 5 | CYP24A1 (rs16999131 (T²⁴⁸R), rs2585427 (p⁻⁴⁶⁴), rs35051736 (R¹⁵⁷Q), rs6022990 (M³⁷⁴T)) |
| | CYP24A1 (rs2762943 (p⁻²⁷⁴) and rs111622401 (p⁻²⁸⁰)) |
| 6 | CYP27A1 (rs2229381 (T¹⁷⁵M) |
| 7 | CYP27B1 (rs13377933, rs2229103, rs28934605, rs440179) |
| | CYP27B1 (rs1l5631506 (p⁻⁴⁰²) and rs11172327 (p⁻⁶⁸¹)) |
| 8 | CYP3A4 (rs2740574 (p⁻²⁸⁵), rs4986907 (R¹⁶²Q), rs4986910, rs28371759) |
| 9 | CYP3A7 (rs45465393, rs45446698 (p⁻²²⁹) |
| 10 | CYP3A43 (rs45621431 (M²⁷⁵I), rs680055 (A³⁴⁰P), rs45558032, rs60530906) |
| 11 | CYP3A5 (rs28365083 (T³⁹⁸N, aka CYP3A5*2 allele), rs776746 (p⁻²³⁷)) |
| 12 | CYP3A7 (rs2257401 (R⁴⁰⁹T), rs45465393, rs45580339) |
| 13 | DMP1 (rs34661425 (K⁴⁶³R)) |
| 14 | FGF23 (rs7955866 (T²³⁹M)) |
| 15 | GC (rs7041 (D⁴³²E) and rs4588 T⁴³⁶K) |
| 16 | GALNT3 (rs13429321 (3′ UTR⁺²⁸²)) |
| 17 | KL (rs9536314 (F³⁵²V) |
| 18 | LRP2 (rs2075252 (K⁴⁰⁹⁴E), rs2229263 (N⁸³S) and rs4667591 (I⁴²¹⁰L)) |
| 19 | PPARGC1A (dbSNP ID rs8192678 (G⁴⁸²S)) |
| 20 | RXRA (rs55836321, rs61751479) |
| 21 | SLC34A1 (rs7379524 (G⁵⁴⁵G) and rs7708314 |
| 22 | SLC24A5 (rs1426654 (T¹¹¹A) |
| 23 | SLC45A2 (rs16891982 (L³⁷⁴F)) |
| 24 | TMEM33 (rs1507086 (3′ UTR⁺¹⁹⁶)) |
| 25 | TRPV5 (rs4236480, rs4252372 (A⁸V), rs4252499 (A⁵⁶³T)) |
| 26 | TRPV6 (rs4987657 (C¹⁹⁷R), rs4987667 (M⁴¹⁸V), rs4987682 (M⁷²¹T)) |
| 27 | VDR (rs2228570, rs61761615, rs11568820) |

In order to reduce the potential for laboratory error and minimize bias in the ancestry-adjusted parameters, genotypes for all variants among African American and matched European American participants were determined simultaneously, and not in separate batches. Each genotyping assay cluster algorithm output was visually inspected and genotyping calls were adjudicated by laboratory personnel blinded to participant characteristics. Samples and genotyping assays with >10% unreadable genotyping calls were excluded from the analysis. For genetic polymorphic variants that did not pass quality assurance, TaqMan assays (ABI, Inc.) were subsequently used to determine genotypes, as listed in Table 1. Vitamin D binding protein (Gc-isoform) diplotype combinations for three major circulating isoforms (Gc-1f, Gc-1s, Gc-2) and one minor Gc-unknown/undetermined category from two variants (GC rs4588 and r57041), as previously described (Wilson et al., 2018, Am J Epidemiol., 187:754-766).

### Statistical Methods

The calcitriol-24,25-dihydroxyvitamin D3-calcifediol proportion ratio (C24CPR) was calculated as:

C24CPR=[Proportion 1,25(OH)₂D₃=(1,25(OH)₂D₃ pmol/L*1000)/((1,25(OH)₂D₃ pmol/L*1000)+25(OH)D₃nmol/L)]

[Proportion 24,25(OH)₂D₃=(24,25(OH)₂D₃ nmol/L)/((24,25(OH)₂D₃ nmol/L)+25(OH)D₃ nmol/L)]

Log-transformed values of three biomarkers (1,25(OH)₂D₃, C24CPR, and the crude ratio (1,25(OH)₂D₃*1000/24,25(OH)₂D₃) were stratified by sex, race/ethnicity and oral contraceptive use and tested for distributional approximation to normality using the Shapiro-Wilk (S-W) test statistic, coefficient of variation (CV %), skewness and kurtosis (SAS PROC Univariate, version 9.4). Spearman's correlation coefficient and the concordance correlation coefficient (CCC) was used to estimate agreement between C24CPR and the crude ratio (Lin L I et al., 1989, Biometrics, 45:255-268). Bivariate differences were determined by paired t-test, Wilcoxon Signed-rank test (median differences) or McNemar's test, as appropriate. Two-sided P-values ≤0.05 were defined as statistically significant.

The χ²-test for deviation from Hardy-Weinberg equilibrium was used to calculate the observed vs. expected distribution of genotypes, stratified by self-reported race/ethnicity which were then compared with the 1000 Genomes genotype frequencies for individuals of European and African descent. Allele frequencies in AA and EA participants in this study were compared with allele frequencies in the 1000 Genomes Project (internationalgenome.org/). In order to better understand the likelihood of inheritance between variants among the individuals included in this study, Lewontin's D′ and the r² statistic were used to estimate linkage disequilibrium between polymorphic variants in the selected models (SAS PROC Allele, Version 9.4, SAS, Inc., Cary, N.C.).

### Multiple Regression Model Building

In order to better approximate a normal distribution, biomarker measurements were natural log-transformed for model building. Associations with non-summer circulating concentrations of 1,25(OH)₂D₃ and C24CPR were tested using multivariable mixed-effect models with a random intercept and restricted maximum likelihood for parameter estimation using paired data (SAS PROC MIXED, version 9.4, SAS, Inc.; (Littell R C et al., 2006, SAS Institute Inc)). The final models were built in three stages, using purposeful model selection principles (Bursac Z et al., 2008, Source Code Biol Med, 3:17). In order to facilitate comparison across models, Stage I of model building identified a common core of adjustment variables, which were then used in Stage II (single genetic variants adjusted for core variables) and finalized in Stage III (multiple genetic variants adjusted for core variables) of model building. Statistically significant variables associated with the C24CPR denominator in a previous analysis (Roff A et al., 2008, Journal of Steroid Biochemistry & Molecular Biology, 112:47-54) were forced into the core adjustment variable set, including: age, dietary vitamin D intake (including supplements and multivitamins), West African genetic ancestry proportion (WAGAP), season (number of days past summer solstice at time of blood draw), and vitamin D binding protein isoforms (Gc-isoforms: Gc-1f, Gc-1s and Gc-2). Additional environmental (non-genetic) adjustment variables were then screened through stepwise selection for each biomarker using SAS PROC REG, version 9.4 (entry p=0.25 and retention p=0.15). The set of final core adjustment variables was identified by using variables that remained statistically significant (p 0.05) in either SAS PROC MIXED (1,25(OH)₂D₃ or C24CPR outcomes) multivariable model.

At Stage III, significant genotypes (p≤0.05) from Stage II were entered (entry p=0.25 and retention p=0.15) into stepwise selection model with forced entry of the core adjustment variables (SAS PROC REG). Stepwise-selected genetic variants were retained if they remained statistically significant in the paired multiple regression (SAS PROC MIXED) model. One-way multiplicative interactions were then tested. Statistically significant interactions with cell sizes less than 5 were noted but not included in the final model. All genetic variants with adjusted p-values ≤0.05 were included in the final model. Adjusted least squares (LS) mean and 95% confidence intervals (CI) based on non-log transformed outcome variables were calculated using the final paired multiple regression models (SAS PROC MIXED). Regression model diagnostics included goodness of fit (R²), Akaike information criterion (AIC)), and collinearity (Durbin-Watson (DW).

### Bioinformatic Tools

LDLink from the National Cancer Institute was used to identify high linkage SNPs within 500 kb for European (EUR) and African (AFR) populations (analysistools.nci.nih.gov/LDlink/). High linkage SNPs were defined as those with both D′ and r² values equal to or greater than 0.80. For significant promoter SNPs, the Gene Transcription Regulation Database (GTRD version 17.11; gtrd.biouml.org/), an index of over 5,000 ChIP-seq experiments was used to identify transcription factor binding sites. Genetic regulatory potential was estimated using RegulomeDB—a publicly available database that uses experimental data sets and computational predictions to identify known and predicted regulatory DNA elements, including regions of DNase hypersensitivity, transcription factor binding sites, and promoter regions that have been biochemically characterized to regulate transcription (regulomedb.org). For significant coding region SNPs, the PHAST conservation score (compgen.cshl.edu/phast/) and SIFT dbSNP (sift.bii.a-star.edu.sg/www/SIFT_dbSNP.html) for predicted activity amino acid substitutions were calculated.

### Study Population

A total of 404 participants enrolled in the study. Genotyping assays did not pass quality control for 22 participants. Of the remaining 382 participants, 188 AA and 188 EA participants could be matched on age (+/−5 years) and sex, with one pair matched only on age (for a total of 376 individuals). Between AA and EA participants, there was no difference in the proportion of other self-reported ancestry groups, corticosteroid use, smoking (prevalence or pack years), diastolic blood pressure, or total serum calcium (Table 2). Dietary vitamin D intake, having an outdoor job, OC use and current tanning bed use was significantly higher among EA. BMI, systolic blood pressure (mmHg), and melanin index were significantly higher among AA. Plasma 1,25(OH)₂D₃ concentrations did not differ between EA and AA women, but were lower among EA men (p=0.038). Mean C24CPR and crude ratio values were approximately two-fold lower for EA compared with AA—with EA women having the lowest mean C24CPR (p<0.001).

TABLE 2
Characteristics by primary self-reported race/ethnicity for healthy African American
and European American young adults matched on age, Central Pennsylvania.

| | African American | | | European American | | | |
|---|---|---|---|---|---|---|---|
| | N = 188 | | | N = 188 | | | |
| Variables | Percent | Mean (std) | | Percent | Mean (std) | | P-value |
| Percent Female | 60.11 | | | 61.17 | | | 0.833 |
| Mean Age | | 21.93 | (4.41) | | 22.30 | (4.50) | 0.419 |
| Self-Reported Race/Ethnicity Groups | | | | | | | |
| African American/African | 96.81 | | | 9.57 | | | <0.001 |
| American Indian/Alaska Native | 16.49 | | | 13.30 | | | 0.385 |
| Asian | 5.32 | | | 6.91 | | | 0.519 |
| European | 22.34 | | | 97.34 | | | <0.001 |
| Vitamin D and Calcium Sources | | | | | | | |
| Vitamin D/Fish Oil Supplements | 13.30 | | | 11.70 | | | 0.641 |
| Dietary Vitamin D Intake (IU/day) | | 208.92 | (195.42) | | 269.98 | (190.42) | 0.003 |
| Total Diet + Supplement (IU/day)^{a} | | 306.39 | (247.13) | | 380.68 | (273.78) | 0.007 |
| Calcium Supplements | 11.43 | | | 13.89 | | | 0.488 |
| Time Spent Outdoors (hours/week) | | 10.45 | (10.28) | | 11.16 | (8.76) | 0.467 |
| Outdoor Job | 28.19 | | | 50.27 | | | <0.001 |
| Tanning Bed Use | 3.72 | | | 23.40 | | | <0.001 |
| Tanning Bed Use (hours/week) | | 0.67 | (0.29) | | 1.13 | (1.09) | 0.084 |
| Oral Contraceptive Use (women) | 19.47 | | | 60.00 | | | <0.001 |
| None | 80.53 | | | 48.70 | | | |
| 0.01 mg to <0.02 mg | 5.31 | | | 5.22 | | | |
| 0.02 mg to <0.03 mg | 7.08 | | | 23.48 | | | |
| ≥0.03 mg | 5.31 | | | 16.52 | | | |
| Unknown Dose | 1.77 | | | 3.48 | | | |
| Medroxyprogesterone acetate use^{b} | 5.31 | | | 2.61 | | | 0.299 |
| Corticosteroid Use | 3.19 | | | 1.60 | | | 0.313 |
| Body Mass Index^{c} | | 26.59 | (6.87) | | 24.40 | (4.23) | 0.001 |
| Current Smoker | 10.11 | | | 10.64 | | | 0.866 |
| Pack years of smoking | | 1.63 | (1.74) | | 2.40 | (2.28) | 0.246 |
| Systolic Blood Pressure (mmHg) | | 115.54 | (11.45) | | 113.21 | (11.60) | 0.051 |
| Diastolic Blood Pressure (mmHg) | | 75.08 | (9.03) | | 73.89 | (7.87) | 0.176 |
| Pre-Hypertension/Hypertension | 44.68 | | | 34.57 | | | |
| Melanin Index Units | | 55.87 | (15.47) | | 29.51 | (5.78) | <0.001 |
| W. African Genetic Ancestry Proportion | | 71.79 | (19.93) | | 9.42 | (9.35) | <0.001 |
| Days from Summer Solstice | | 86.16 | (35.82) | | 96.20 | (24.06) | 0.002 |
| 25(OH)D₃ (nmol/L) | | | | | | | |
| Men | | 45.63 | (28.18) | | 66.00 | (25.21) | <0.001 |
| Women | | 50.01 | (31.00) | | 109.58 | (58.43) | <0.001 |
| 24,25(OH)₂D₃ (nmol/L) | | | | | | | |
| Men | | 4.80 | (4.84) | | 7.53 | (3.51) | 0.001 |
| Women | | 5.40 | (5.83) | | 15.81 | (11.13) | <0.001 |
| 1,25(OH)₂D₃ (pmol/L) | | | | | | | |
| Men | | 87.61 | (30.12) | | 78.31 | (23.49) | 0.038 |
| Women | | 103.30 | (36.67) | | 106.94 | (40.13) | 0.475 |
| Crude ratio 1,25(OH)₂D₃ | | | | | | | |
| to 24,25(OH)₂D₃ | | | | | | | |
| Men | | 31.34 | (28.47) | | 13.30 | (11.43) | <0.001 |
| Women | | 35.06 | (32.05) | | 10.10 | (12.10) | <0.001 |
| C24CP Ratio | | | | | | | |
| Men | | 8.97 | (4.59) | | 5.80 | (2.36) | <0.001 |
| Women | | 9.10 | (4.83) | | 4.66 | (2.53) | <0.001 |
| Serum Total Calcium (mg/dL) | | | | | | | |
| Men | | 10.40 | (0.77) | | 10.39 | (1.19) | 0.973 |
| Women | | 10.09 | (0.81) | | 10.02 | (1.29) | 0.602 |

^{a}Includes intake from vitamin D supplements and multivitamins,
^{b}All women taking medroxyprogesterone acetate (MPA) were also taking oral contraceptives,
^{c}kg/m².

### Biomarker Correlation and Distributional Characteristics

C24CPR was not highly correlated with 1,25(OH)₂D₃ (rho=0.14, p=0.008), but was highly correlated with the crude ratio (1,25(OH)₂D₃ to 24,25(OH)₂D₃) (rho=0.96; Table 3).

TABLE 3
Correlation between plasma vitamin D metabolite concentrations among healthy young
adult African American and European American participants. Correlation calculated
with Spearman's rho values, including respective 95% confidence intervals (in
parenthesis). All correlations calculated with log-transformed values.

| | | | | Crude Ratio |
|---|---|---|---|---|
| | | | | 1,25(OH)₂D₃ to |
| | 25(OH)D₃ | 1,25(OH)₂D₃ | 24,25(OH)₂D₃ | 24,25(OH)₂D₃ |
| 1,25(OH)₂D₃ | 0.12 | — | | |
| | (0.02, 0.22) | | | |
| 24,25(OH)₂D₃ | 0.90 | 0.15 | — | |
| | (0.88, 0.92) | (0.05, 0.25) | | |
| Crude Ratio 1,25(OH)₂D₃ to | −0.81 | 0.26 | −0.89 | — |
| 24,25(OH)₂D₃ | (−0.84, −0.77) | (0.16-0.35) | (−0.91, −0.87) | |
| C24CPR | −0.72 | 0.14 | −0.90 | 0.96 |
| | (−0.76, −0.66) | (0.04, 0.23) | (−0.92, −0.88) | (0.95, 0.96) |

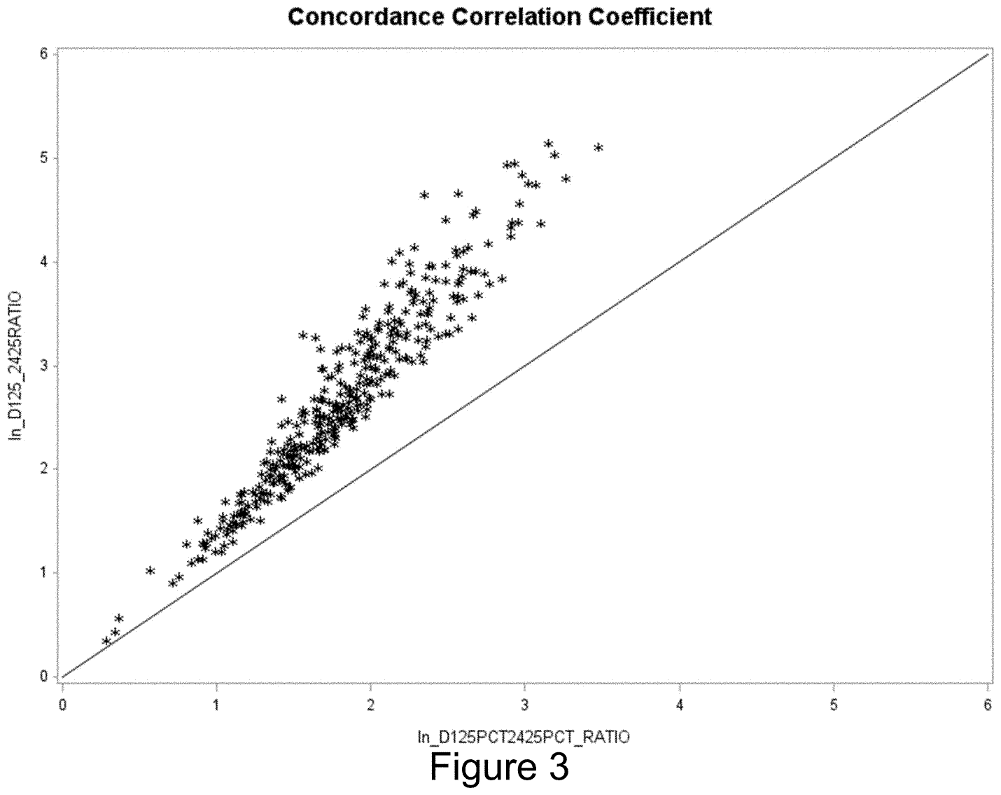

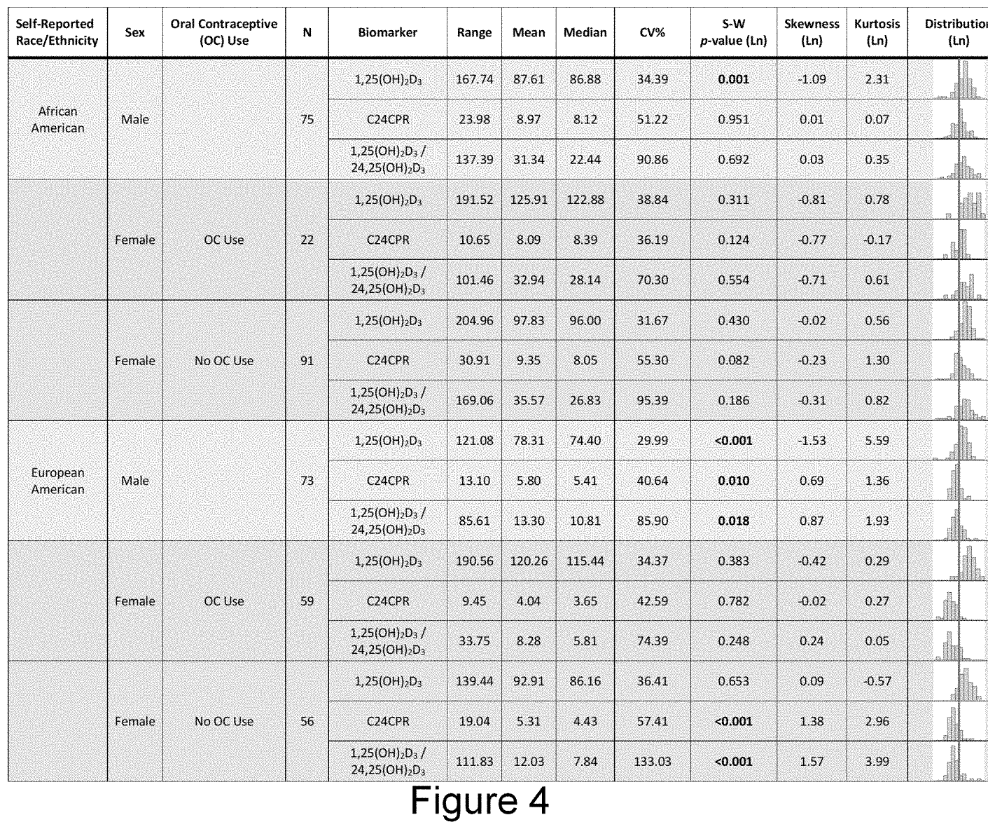

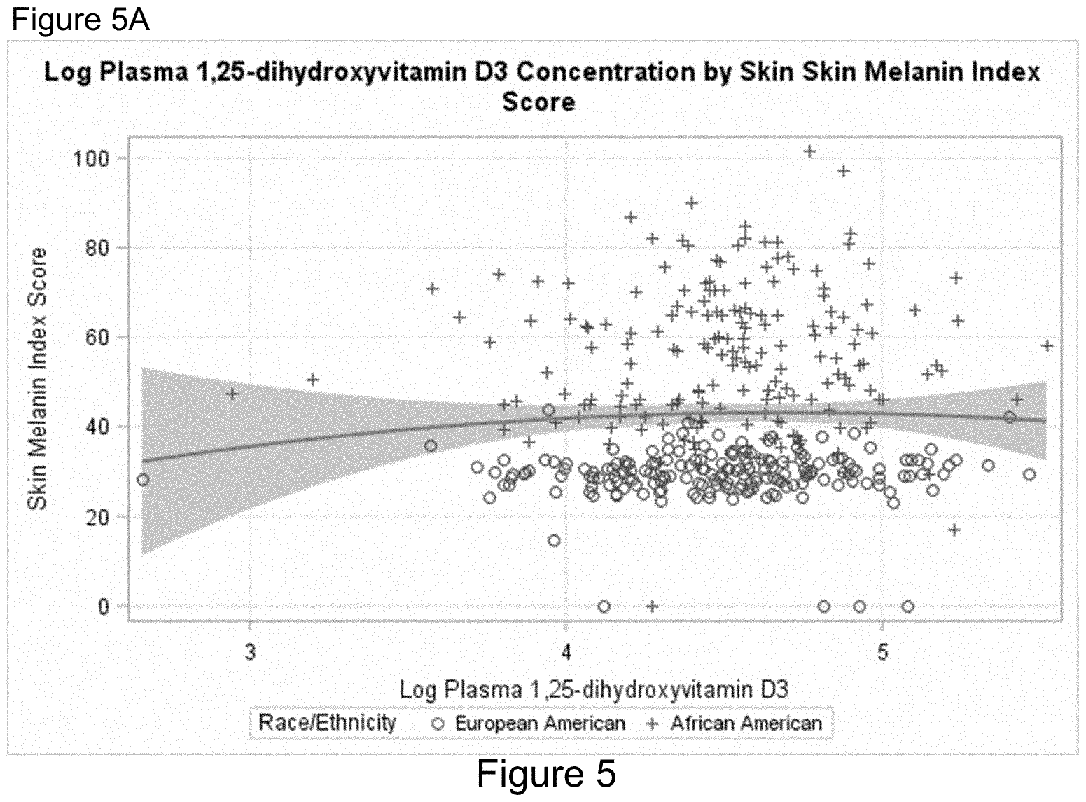

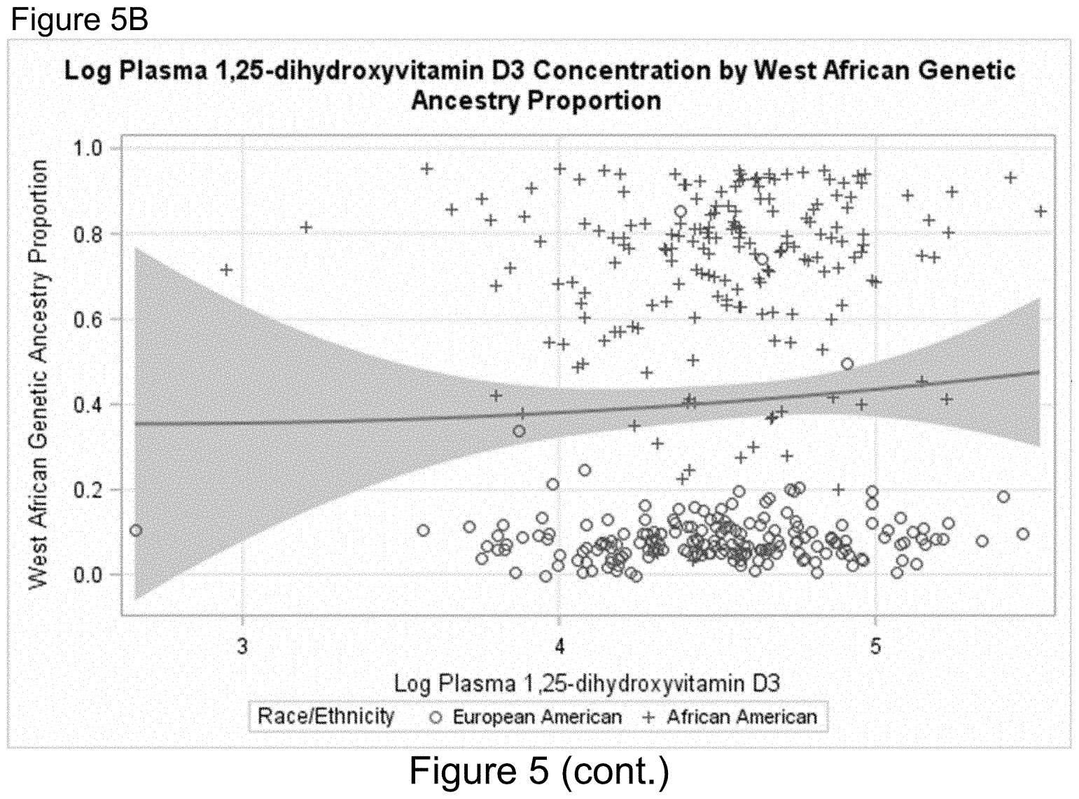

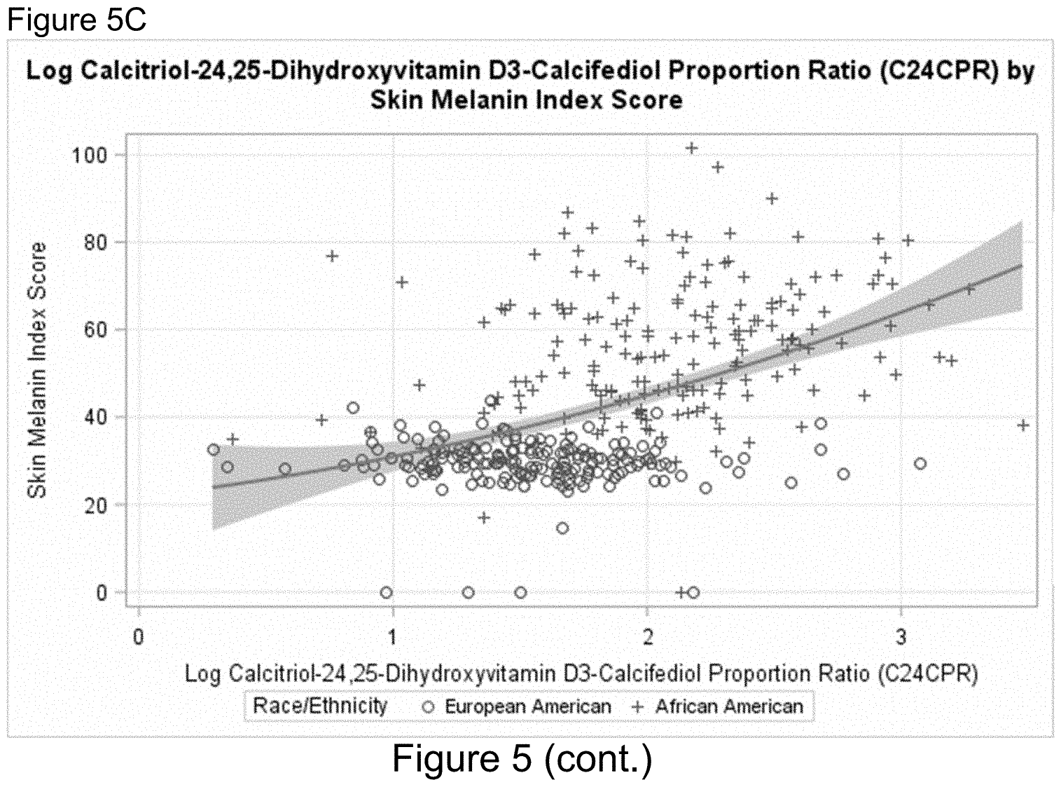

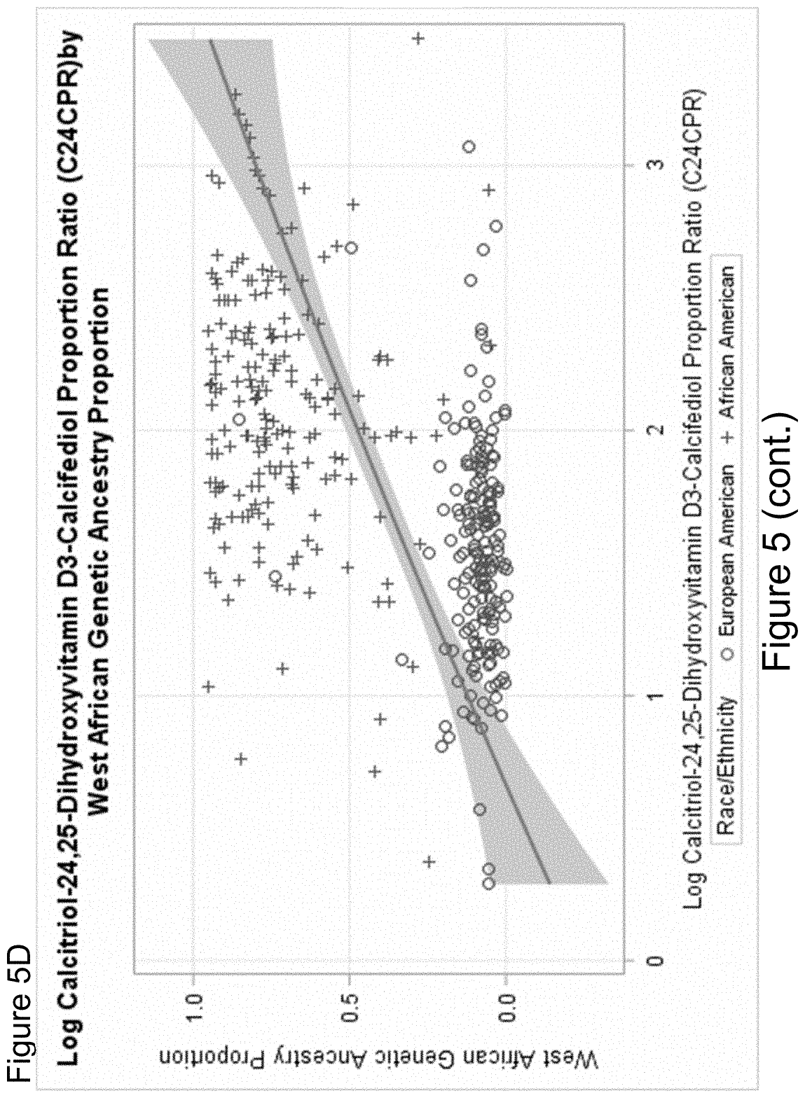

However, agreement between C24CPR and the crude ratio was lower due to greater dispersion of the crude ratio at higher values (CCC=0.49, 95% CI: 0.45, 0.53 (log-transformed); CCC=0.22; 95% CI: 0.20-0.24 (non-log transformed), FIG. 3). Comparing the distributional characteristics of the C24CPR to the crude ratio, S-W tests for normality were similar for each population subgroup (FIG. 4). In contrast, CV % for C24CPR were roughly half and measures of skewness and kurtosis were closer to a normal distribution for four of the six sub-groups compared with the crude ratio. Both skin melanin index and WAGAP were individually poorly correlated with 1,25(OH)₂D₃ (rho=0.05 and 0.10, respectively), and moderately correlated with C24CPR (rho=0.49 and 0.51, respectively) (FIG. 5A through FIG. 5D).

### Expected Genotype Frequencies

Among AA, HWE tests for equilibrium were rejected for SLC45A2 (rs16891982), CYP27A1 (rs2229381), LRP2 (rs4667591), TRPV6 (rs4987667) and Gc-2 isoform (Table 4).

TABLE 4
Allele Frequencies and Hardy-Weinberg Equilibrium (HWE) among African American
(AA) and European American (EA) Participants (Central Pennsylvania) compared
with 1000 Genomes African (AFR) and European (EUR) Populations.

| | | | AA Major | AA | | EA Major | EA | AA- | | AFR- |
|---|---|---|---|---|---|---|---|---|---|---|
| | Allele | AA | allele | HWE | EA | Allele | HWE | EA | AFR | EUR |
| Locus | Maj/Min | Count | Freq | p-value^{a} | Count | Freq | p-value^{a} | (Δ) | Freq | (Δ)^{b} |
| CYP27B1 | G/C | 355/21 | 0.94 | 0.107 | 374/0 | 1.00 | — | −0.06 | 0.96 | −0.04 |
| rs115631506 | | | | | | | | | | |
| (p⁻⁴⁰⁵) | | | | | | | | | | |
| CYP2R1 | G/A | 265/107 | 0.71 | 0.471 | 251/123 | 0.67 | 0.870 | 0.04 | 0.78 | 0.16 |
| rs10741657 | | | | | | | | | | |
| (P⁻¹¹²⁷) | | | | | | | | | | |
| CYP27B1 | G/A | 358/18 | 0.95 | 0.063 | 373/1 | 0.99 | 1.000 | −0.04 | 0.93 | −0.07 |
| rs11172327 | | | | | | | | | | |
| (p⁻⁶⁸¹) | | | | | | | | | | |
| SLC45A2 | C/G | 306/70 | 0.81 | <0.001 | 26/348 | 0.07 | 1.000 | 0.74 | 0.96 | 0.90 |
| rs16891982 | | | | | | | | | | |
| (L³⁷⁴F) | | | | | | | | | | |
| CALB1 | C/T | 279/95 | 0.75 | 0.845 | 245/123 | 0.67 | 0.420 | 0.08 | 0.72 | 0.01 |
| rs2142093 | | | | | | | | | | |
| (p⁻⁴¹⁰) | | | | | | | | | | |
| CALB1 | A/T | 237/139 | 0.63 | 1.000 | 246/128 | 0.66 | 0.332 | −0.03 | 0.57 | −0.14 |
| rs2142094 | | | | | | | | | | |
| (p⁻⁵⁰²) | | | | | | | | | | |
| CYP27A1 | C/T | 372/4 | 0.99 | 0.017 | 374/0 | 1.00 | — | −0.01 | 0.98 | −0.01 |
| rs2229381 | | | | | | | | | | |
| (T¹⁷⁵M) | | | | | | | | | | |
| VDR | G/A | 275/101 | 0.73 | 0.581 | 241/133 | 0.64 | 0.640 | 0.09 | 0.81 | 0.19 |
| rs2228570 | | | | | | | | | | |
| (M¹T) | | | | | | | | | | |
| CYP3A7 | G/C | 195/181 | 0.52 | 0.664 | 346/28 | 0.93 | 0.604 | −0.41 | 0.38 | −0.55 |
| rs2257401 | | | | | | | | | | |
| (R⁴⁰⁹T) | | | | | | | | | | |
| CYP24A1 | G/T | 366/10 | 0.97 | 1.000 | 350/24 | 0.94 | 0.548 | 0.03 | 0.99 | 0.08 |
| rs2762943 | | | | | | | | | | |
| (p⁻²⁶¹) | | | | | | | | | | |
| CYP3A43 | G/A | 372/4 | 0.99 | 1.000 | 357/17 | 0.95 | 1.000 | 0.04 | 0.99 | 0.02 |
| rs45621431 | | | | | | | | | | |
| (M²⁷⁵I) | | | | | | | | | | |
| LRP2 | T/G | 264/110 | 0.71 | 0.003 | 66/306 | 0.18 | 1.000 | 0.53 | 0.85 | 0.64 |
| rs4667591 | | | | | | | | | | |
| (I⁴²¹⁰L) | | | | | | | | | | |
| CYP3A4 | G/A | 368/4 | 0.99 | 1.000 | 374/0 | 1.00 | — | −0.01 | 0.98 | −0.01 |
| rs4986907 | | | | | | | | | | |
| (R¹⁶²Q)c | | | | | | | | | | |
| TRPV6 | A/G | 250/126 | 0.66 | <0.001 | 345/29 | 0.92 | 0.598 | −0.26 | 0.42 | −0.50 |
| rs4987667 | | | | | | | | | | |
| (M⁴¹⁸V) | | | | | | | | | | |
| CYP3A43 | G/C | 255/119 | 0.68 | 0.401 | 353/21 | 0.94 | 0.455 | −0.26 | 0.62 | −0.33 |
| rs680055 | | | | | | | | | | |
| (p^{-340A})^{d} | | | | | | | | | | |
| PPARGC1A | G/A | 336/40 | 0.89 | 0.136 | 241/133 | 0.64 | 0.749 | 0.25 | 0.95 | 0.31 |
| rs8192678 | | | | | | | | | | |
| (G⁴⁸²S) | | | | | | | | | | |
| KL | T/G | 304/72 | 0.81 | 1.000 | 329/43 | 0.88 | 1.000 | −0.07 | 0.80 | −0.01 |
| rs9536314 | | | | | | | | | | |
| (F³⁵²V) | | | | | | | | | | |
| GC (Gc-2 | 0/1 | 330/46 | 0.88 | 0.039 | 265/105 | 0.72 | 0.071 | 0.16 | 0.87 | 0.14 |
| Allele)^{e} | | | | | | | | | | |
| GC (Gc-1f | 1/0 | 250/126 | 0.66 | 1.000 | 59/317 | 0.16 | 0.089 | 0.50 | 0.68 | 0.54 |
| Allele)^{e} | | | | | | | | | | |

^{a}Exact p-values from HWE test (SAS Proc Allele, version 9.4),
^{b}AFR and EUR single nucleotide variant frequencies derived from 1000 Genomes population frequencies available at ncbi.nlm.nih.gov/SNP/. Bolded values are highest comparative delta value between Pennsylvania (AA-EA) and the reference (AFR-EUR) from 1000 genomes (i.e., at a 50 percent difference or higher).
^{c}Also known as the CYP3A4*15 allele.
^{d}Also known as the CYP3A43*3 allele.
^{e}Comparative Gc-isoform allele frequencies for AFR and EUR are derived from Kamboh (1986) for Pennsylvania.

Among EA, no genetic variants were out of HWE and two variants had no minor allele (CYP27A1 rs2229381 and CYP3A4 rs4986907). Consistent with greater admixture in US population groups, AA minus EA allele frequency delta values among the study population were slightly lower than AFR-EUR reference delta values.

### Multiple Regression Model Building

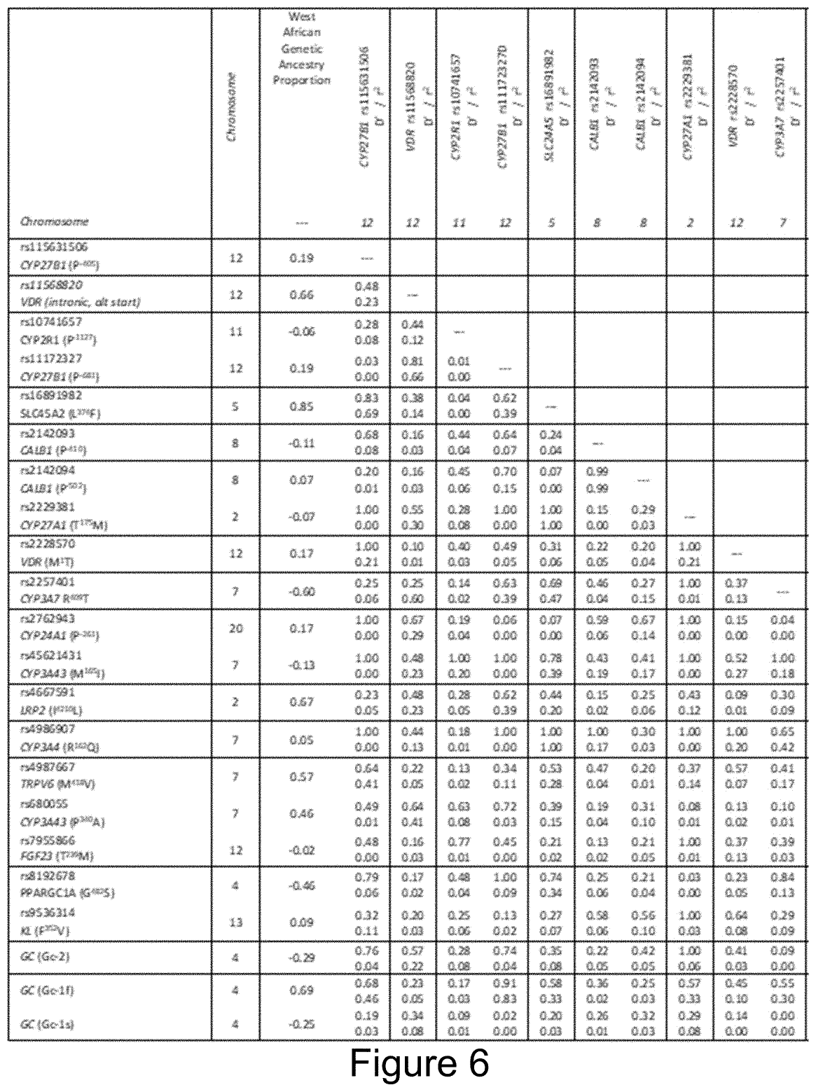

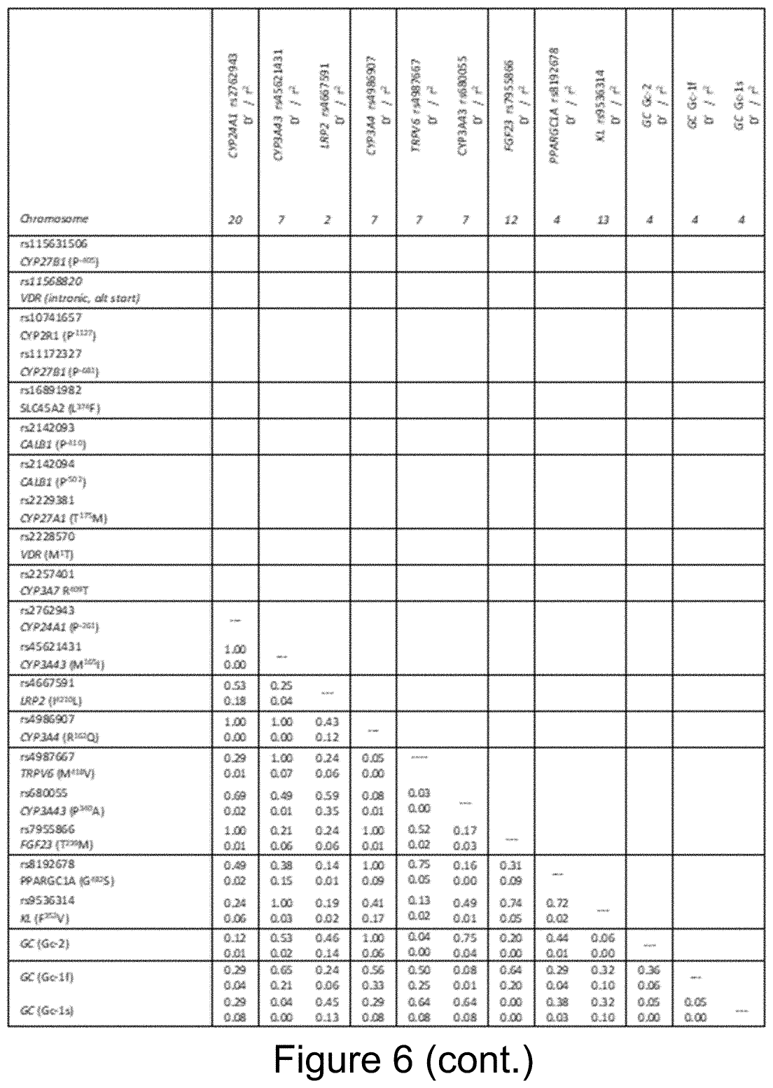

At Stage I of model building, core adjustment variables retained after stepwise selection included: age, body mass index class, GC-isoform, current OC and medroxyprogesterone acetate use, season (days from summer solstice at blood draw date), sex, smoking (pack years), systolic blood pressure (mmHg), tanning bed use (past 30 days), time of day of blood draw, vitamin D intake from diet and supplements (including multi-vitamins) and WAGAP. At Stage II of model development, CYP24A1 rs2762943, CYP3A4 rs4986907 (R¹⁶²Q), CYP3A43 rs45621431 (M²⁷⁵1), CYP3A43 rs680055 (A³⁴⁰P), FGF23 rs7955866 (T²³⁹M), KL rs9536314 (F³⁵²V), LRP2 rs4667591 (I⁴²¹⁰L), SLC45A2 rs16891982, TRPV6 rs4987667, and VDR rs11568820, were independently associated with C24CPR; whereas CYP27A1 rs2229381, CYP27B1 rs115631506, CALB1 (r521429093 and r52142094), CYP3A7 rs2257401, and PPARGC1A rs8192678 were independently associated with 1,25(OH)₂D₃. Among significant Stage II variants, those with high pairwise LD included CALB1 rs2142093/CALB1 rs2142094, CYP3A4 rs4986907/SLC45A2 rs16891982; and CYP27A1 rs2229381/SLC45A2 rs16891982 (FIG. 6).

### Final Models

In the final C24CPR model (Stage III), genetic factors WAGAP, VDR rs2228570, CYP3A43 rs45621431, KL rs9536314, and FGF23 rs7955866 remained significant (Table 5).

TABLE 5
Genetic variants significantly associated with C24CPR and calcitriol
(1,25(OH)₂D₃) with adjusted least squares mean, 95% confidence interval (CI) and R².

| | | Mean | | | Mean | | |
|---|---|---|---|---|---|---|---|
| | | C24CPR | | | 1,25(OH)₂D₃ | | |
| Variable | N | (95% CI) | p-value^{a} | R^{2 b} | (95% CI) | p-value^{a} | R^{2 b} |
| West African Genetic Ancestry Proportion | | | | 0.282 | | | 0.006 |
| 0.00 to 0.24 | 191 | 5.80 (5.14- | Ref | | 91.32 (85.25- | Ref | |
| | | 6.45) | | | 97.38) | | |
| 0.25 to 0.49 | 22 | 7.75 (6.20- | 0.050 | | 96.06 (82.62- | 0.657 | |
| | | 9.31) | | | 109.50) | | |
| 0.50 to 0.74 | 57 | 7.75 (6.72- | <0.001 | | 99.43 (90.36- | 0.156 | |
| | | 8.78) | | | 108.51) | | |
| 0.75 to 1.00 | 106 | 8.89 (8.02- | <0.001 | | 105.36 (97.23- | 0.022 | |
| | | 9.77) | | | 113.50) | | |
| p-trend | | | <0.001 | | | 0.025 | |
| Gc-Isoform^{c} (Vitamin D Binding Protein) | | | | 0.187 | | | 0.054 |
| Gc-2/Gc-2 | 26 | 6.49 (5.07- | Ref | | 78.00 (65.24- | Ref | |
| | | 7.92) | | | 90.77) | | |
| Gc-2/Gc-1f | 40 | 6.87 (5.70- | 0.382 | | 93.31 (83.15- | 0.019 | |
| | | 8.04) | | | 103.47) | | |
| Gc-2/Gc-1s^{d} | 59 | 7.18 (6.14- | 0.487 | | 86.52 (77.51- | 0.181 | |
| | | 8.22) | | | 95.53) | | |
| Gc-1f/Gc-1f | 91 | 7.50 (6.60- | 0.215 | | 104.65 (96.69- | <0.001 | |
| | | 8.40) | | | 112.61) | | |
| Gc-1s/Gc-1s | 68 | 7.07 (6.10- | 0.634 | | 103.66 (95.16- | <0.001 | |
| | | 8.05) | | | 112.16) | | |
| Gc-1f/Gc-1s | 87 | 6.75 (5.97- | 0.918 | | 98.44 (91.48- | 0.003 | |
| | | 7.52) | | | 105.41) | | |
| p-additive (Gc- | | | 0.150 | | | <0.001 | |
| 2 allele) | | | | | | | |
| VDR (dbSNP ID rs2228570, T¹M) | | | | 0.017 | — | | |
| GG (T¹T or | 181 | 5.51 (4.36- | Ref | | — | — | |
| short/short) | | 6.66) | | | | | |
| GA (T¹M or | 154 | 6.99 (6.43- | 0.305 | | — | — | |
| short/long) | | 7.56) | | | | | |
| AA (M¹M or | 40 | 7.53 (7.00- | 0.002 | | — | — | |
| long/long) | | 8.05) | | | | | |
| p-additive | | | 0.005 | | | — | |
| CYP3A43 (dbSNP ID rs45621431, M²⁷⁵I) | | | | 0.033 | | | — |
| GG (M²⁷⁵M) | 354 | 7.17 (6.80- | Ref | | — | — | |
| | | 7.54) | | | | | |
| AG (M²⁷⁵I) | 21 | 5.67 (4.03- | 0.021 | | — | — | |
| | | 7.31) | | | | | |
| AA (I²⁷⁵I) | 0 | — | — | | — | — | |
| p-additive | | | 0.020 | | | — | |
| KL (Klotho dbSNP ID rs9536314, F³⁵²V) | | | | 0.031 | | | — |
| TT (F³⁵²F) | 268 | 7.03 (6.60- | Ref | | — | — | |
| | | 7.46) | | | | | |
| TG (F³⁵²V) | 97 | 6.85 (6.13- | 0.841 | | — | — | |
| | | 7.58) | | | | | |
| GG (V³⁵²V) | 9 | 12.00 (9.53- | 0.016 | | — | — | |
| | | 14.47) | | | | | |
| p-additive | | | 0.121 | | | — | |
| FGF23 (dbSNP ID rs7955866, T²³⁹M) | | | | 0.008 | | | — |
| CC (T²³⁹T) | 301 | 7.23 (7.19- | Ref | | — | — | |
| | | 15.17) | | | | | |
| CT (T²³⁹M) | 65 | 6.35 (5.46- | 0.022 | | — | — | |
| | | 7.25) | | | | | |
| TT (M²³⁹M) | 3 | 11.18 (6.82- | 0.151 | | — | — | |
| | | 7.63) | | | | | |
| p-additive | | | 0.142 | | | — | |
| CYP27B1 (dbSNP ID rs115631506, p⁻⁴⁰⁵)^{e} | | | | — | | | 0.026 |
| GG | 356 | — | — | | 97.33 (94.16- | Ref | |
| | | | | | 100.49) | | |
| GC | 17 | — | — | | 82.00 (66.78- | 0.006 | |
| | | | | | 97.20) | | |
| CC | 2 | — | — | | 131.72 (88.48- | 0.189 | |
| | | | | | 174.95) | | |
| p-additive | | | — | | | 0.137 | |
| CYP3A7 (dbSNP ID rs2257401, R⁴⁰⁹T) | | | | — | | | 0.008 |
| GG (R⁴⁰⁹R) | 204 | — | — | | 99.47 (94.58- | Ref | |
| | | | | | 104.36) | | |
| GC (R⁴⁰⁹T) | 119 | — | — | | 90.83 (84.80- | 0.036 | |
| | | | | | 96.86) | | |
| CC (T⁴⁰⁹T) | 52 | — | — | | 99.96 (90.28- | 0.912 | |
| | | | | | 109.64) | | |
| p-additive | | | — | | | 0.650 | |
| PPARGC1A (dbSNP ID rs8192678, G⁴⁸²S) | | | | — | | | |
| GG (G⁴⁸²G) | 228 | — | — | | 97.30 (93.05- | Ref | 0.022 |
| | | | | | 101.55) | | |
| GA (G⁴⁸²S) | 121 | — | — | | 99.07 (93.14- | 0.519 | |
| | | | | | 104.99) | | |
| AA (S⁴⁸²S) | 26 | — | — | | 81.50 (68.96- | 0.007 | |
| | | | | | 94.03) | | |
| p-additive | | | — | | | 0.127 | |
| Interaction Effects | | | | | | | |
| CALB1 (dbSNP ID rs2142094, P⁻⁵⁰²) * GC | | | | — | | | 0.019 |
| AA * Gc-1f/Gc- | 41 | — | — | | 118.09 (107.49- | Ref | |
| 1f | | | | | 128.68) | | |
| AT * Gc-1f/Gc- | 37 | — | — | | 92.84 (82.00- | 0.002 | |
| 1f | | | | | 103.69) | | |
| TT * Gc-1f/Gc- | 13 | — | — | | 90.83 (73.22- | 0.010 | |
| 1f | | | | | 108.43) | | |
| AA * All other | 118 | — | — | | 97.22 (91.26- | 0.004 | |
| isoforms | | | | | 103.18) | | |
| AT * All other | 128 | — | — | | 90.35 (84.72- | <0.001 | |
| isoforms | | | | | 95.98) | | |
| TT * All other | 38 | — | — | | 100.83 (90.70- | 0.016 | |
| isoforms | | | | | 110.96) | | |
| p-interaction | | | — | | | 0.009 | |
| R² = Total/Genetic/Environment | | 0.495/0.310/0.304 | | | 0.374/0.121/0.241 | | |
| Durbin-Watson | | 1.781 | | | 1.909 | | |

^{a}P-values and least squares mean values calculated using paired regression modeling with SAS Proc Mixed (version 9.4) and log transformed and non-log transformed blood biomarker values, respectively. All models are adjusted for age, body mass index class, GC-isoform, current oral contraceptive and medroxyprogesterone acetate use, season (days from summer solstice at blood draw date), sex, smoking (pack years), systolic blood pressure (mmHg), tanning bed use (past 30 days), time of day of blood draw (quartile), vitamin D intake from diet and supplements (including multi-vitamins, IU/d), West African Genetic Ancestry Proportion (WAGAP), and only those other genetic polymorphisms with listed values in the table (including the interaction term). Calcitriol concentrations are measured in pmol/L. Model variable Spearman bivariate correlations do not exceed rho = 0.36. For purposes of comparison, values for Gc-isoform in the 1,25(OH)₂D₃ model are derived from the full model without the interaction with CALB1.
^{b} R² values are calculated using SAS Proc GLM for the full final models, genetic variables only, environmental variables only (age, body mass index class, current oral contraceptive and medroxyprogesterone acetate use, season (days from summer solstice at blood draw date), sex, smoking (pack years), systolic blood pressure (mmHg), tanning bed use (past 30 days), time of day of blood draw (quartile), vitamin D intake from diet and supplements (including multi-vitamins, IU/d)), and each variable in the table independently.
^{c}Gc (aka Vitamin D Binding Protein) Isoform predicted from rs7041/rs4588 genotype combinations as follows: Gc-1f/Gc-1f = 432^{Asp/Asp} (TT)/436^{Thr/Thr} (CC); Gc-1f/Gc-2 = 432^{Asp/Asp} (TT)/436^{Thr/Lys} (CA); Gc-2/Gc-2 = 432^{Asp/Asp} (TT)/436^{Lys/Lys} (AA); Gc-1f/Gc-1s = 432^{Asp/Glu} (TG)/436^{Thr/Thr} (CC); Gcls/Gc-2** = 432^{Asp/Glu} (TG)/436^{Thr/Lys} (CA); Gc-ls/Gc-1s = 432^{Glu/Glu} (GG)/436^{Thr/Thr} (CC).
^{d}Gc-1s/Gc-2** is a hypothetical Gc-isoform combination for the double heterozygote genotype combination (rs4588 = “CA” and rs7041 = “TG”) group. The alternative combination is Gc-1f/undefined. Therefore, adjustment categories include a dummy variable for Gc-isoform unknown/undefined.
^{e}p⁻⁴⁰⁵ indicates promoter region, at 405 base pairs upstream of the start site.

In the final 1,25(OH)₂D₃ model, genetic factors GC (with Gc-2 having lowest 1,25(OH)₂D₃ concentration), WAGAP, CYP27B1 rs115631506, CYP3A7 rs2257401, PPARGC1A rs8192678, and CALB1 rs2142094 remained significant, as well as an interaction between Gc-lf and CALB1 rs2142094 (p_{interaction}=0.009). The final models explained approximately half of the variation in the log C24CPR model (R²=0.495), and one third of the variation in 1,25(OH)₂D₃ (R²=0.374). WAGAP had the greatest R² value of any single variable considered (R²=0.282, C24CPR), including environmental variables (Table 6).

TABLE 6
Calcitriol-24,25-dihydroxyvitamin D3-calcifediol proportion ratio (C24CPR) and calcitriol
(1,25(OH)₂D₃) adjusted least squares mean by demographic and environmental factors.

| | | Mean | | | Mean | | |
|---|---|---|---|---|---|---|---|
| | | C24CPR | | | 1,25(OH)₂D₃ | | |
| Variable | N | (95% CI) | p-value^{a} | R²_{adj}^{b} | (95% CI) | p-value^{a} | R²_{adj}^{b} |
| Sex | | | | 0.014 | | | 0.089 |
| Men | 148 | 7.47 (6.79- | Ref | | 90.89 (85.11- | Ref | |
| | | 8.15) | | | 96.66) | | |
| Women | 228 | 6.85 (6.33- | 0.179 | | 100.67 (96.24- | 0.006 | |
| | | 7.37) | | | 105.09) | | |
| Smoking | | | | 0.007 | | | 0.022 |
| Non- | 355 | 7.04 (6.67- | Ref | | 97.42 (94.24- | Ref | |
| Smokers | | 7.41) | | | 100.60) | | |
| >0-9 Pack | 12 | 8.46 (6.34- | 0.417 | | 95.37 (77.10- | 0.943 | |
| Years | | 10.57) | | | 113.63) | | |
| >9 Pack | 9 | 7.32 (4.92- | 0.838 | | 74.86 (53.88- | 0.004 | |
| Years | | 9.72) | | | 95.84) | | |
| p-trend | | | 0.265 | | | 0.003 | |
| Tanning Bed | | | | 0.094 | | | 0.042 |
| Use | | | | | | | |
| No | 325 | 7.23 (6.84- | Ref | | 93.93 (90.57- | Ref | |
| | | 7.63) | | | 97.29) | | |
| Yes | 51 | 6.18 (5.08- | 0.007 | | 115.64 (106.10- | <0.001 | |
| | | 7.27) | | | 125.17) | | |
| Days from Summer Solstice (median cut off) | | | | 0.051 | | | <0.001 |
| Below (26 | 187 | 7.79 (7.25- | Ref | | 93.79 (89.27- | Ref | |
| to 93) | | 8.32) | | | 98.30) | | |
| Above(94 | 189 | 6.41 (5.88- | 0.006 | | 99.79 (95.31- | 0.097 | |
| to 173) | | 6.94) | | | 104.28) | | |
| p-trend | | | <0.001 | | | 0.248 | |
| Oral Contraceptive Ethinyl Estrogen (EE)^{c} | | | | 0.112 | | | 0.121 |
| None | 147 | 7.32 (6.90- | Ref | | 96.76 (91.01- | Ref | |
| | | 7.75) | | | 102.52) | | |
| 0.01 | 12 | 6.34 (3.64- | 0.409 | | 127.75 (102.80- | 0.017 | |
| to <0.02 mg | | 9.05) | | | 152.71) | | |
| 0.02 | 35 | 5.94 (4.71- | 0.003 | | 130.23 (118.25- | <0.001 | |
| to <0.03 mg | | 7.17) | | | 142.21) | | |
| ≥0.03 mg | 25 | 6.34 (4.83- | 0.022 | | 115.29 (100.81- | 0.072 | |
| | | 7.85) | | | 129.77) | | |
| p-trend | | | <0.001 | | | <0.001 | |
| EE plus | 9 | 7.47 (5.15- | 0.722 | | 99.52 (76.70- | 0.742 | |
| MPA | | 9.78) | | | 122.34) | | |
| Unknown | 5 | 5.67 (2.53- | 0.128 | | 126.35 (95.19- | 0.059 | |
| dose | | 8.82) | | | 157.52) | | |
| Time of Blood | | | | 0.036 | | | 0.032 |
| Draw^{d} | | | | | | | |
| 08:00 to | 92 | 6.60 (5.84- | Ref | | 90.05 (83.51- | Ref | |
| 10:29 | | 7.35) | | | 96.59) | | |
| 10:30 to | 94 | 6.75 (6.03- | 0.208 | | 95.73 (89.44- | 0.164 | |
| 11:54 | | 7.48) | | | 102.03) | | |
| 11:55 to | 94 | 6.72 (6.00- | 0.355 | | 102.04 (95.79- | 0.020 | |
| 13:49 | | 7.43) | | | 108.28) | | |
| 13:50 to | 94 | 8.28 (7.53- | 0.003 | | 99.04 (92.57- | 0.067 | |
| 16:30 | | 9.03) | | | 105.50) | | |
| p-trend | | | | | | 0.037 | |
| Body Mass | | | | 0.048 | | | 0.035 |
| Index^{e} | | | | | | | |
| Under | 9 | 9.56 (7.18- | 0.159 | | 119.41 (98.78- | 0.252 | |
| Weight | | 11.94) | | | 140.05) | | |
| Normal | 209 | 6.76 (6.26- | Ref | | 100.47 (96.16- | Ref | |
| Weight | | 7.26) | | | 104.78) | | |
| Overweight | 98 | 6.91 (6.19- | 0.506 | | 92.48 (86.15- | 0.031 | |
| | | 7.63) | | | 98.81) | | |
| Class I | 31 | 8.17 (6.89- | 0.004 | | 89.88 (78.89- | 0.012 | |
| Obesity | | 9.44) | | | 100.87) | | |
| Class II | 16 | 9.59 (7.71- | 0.004 | | 88.45 (72.52- | 0.180 | |
| Obesity | | 11.47) | | | 104.38) | | |
| Class III | 11 | 6.50 (4.39- | 0.847 | | 80.25 (61.49- | 0.034 | |
| Obesity | | 8.62) | | | 99.02) | | |
| p-trend | | | 0.017 | | | <0.001 | |

^{a}P-values and least squares mean values are from paired regression modeling using SAS Proc Mixed (version 9.4) using log transformed and non-log transformed blood biomarker values, respectively. All models are adjusted for age, sex, systolic blood pressure (mmHg), pack years of smoking, tanning bed use in the past 30 days, West African genetic ancestry proportion, current oral contraceptive and medroxyprogesterone acetate use, vitamin D intake from diet and supplements (including multi-vitamins), days from summer solstice at time of blood draw, time of day of blood draw, body mass index class, and other genetic polymorphisms with listed values in the table (including the interaction term). Calcitriol measures are in pmol/L. Model variable Spearman correlations do not exceed rho = 0.36.
^{b}Adjusted R² values calculated by SAS Proc GLM for each variable independently.
^{c}LS means and p-trend values calculated among women only for each daily dose. MPA = medroxyprogesterone acetate. All women taking medroxyprogesterone acetate (MPA) were also taking oral contraceptives and therefore the category “EE plus medroxyprogesterone acetate” included as an adjustment variable in the model. P-trend calculated using dose as a categorical variable (excluding unknown and EE plus medroxyprogesterone acetate categories).
^{d}Hours 0800 to 1630. P-trend calculated as a categorical variable.
^{e}Body Mass Index (BMI) Class defined according to the World Health Organization, under-weight (<18.5 kg/m²), normal weight (18.5-24.9 kg/m²), overweight (25.0-29.9 kg/m²), Class I obesity (30.0-34.9 kg/m²), class II obesity (35.0-39.9 kg/m²), class III obesity (≥40.0 kg/m²). BMI missing for two subjects and classified as unknown. P-trend calculated for BMI as a categorical variable.

Statistically significant interactions not included (i.e., cell sizes <5) were Gc-1s*rs115631506 (p-interaction=0.030) and PPARGC1A rs8192678*smoking (p-interaction=0.008), with respect to 1,25(OH)₂D₃ model and VDR rs2228570*time of day (p-interaction=0.022), with respect to C24CPR (data not shown). Both final models passed tests for multicollinearity (D-W values below 2.0 and Spearman's rho values all below 0.36).

### Bioinformatics

Variants predicted as likely to affect DNA binding from RegulomeDB included: CYP27B1 rs115631506; ARPC1A rs150359997 (highly linked to CYP3A43 r545621431), and CYP3A7 rs61017966 (highly linked to CYP3A7 rs2257401). SIFT prediction scores for CYP3A43 rs45621431, KL rs9536314 and VDR rs2228570 were classified as ‘deleterious’.

### C24CPR as a Metric of Vitamin D Status

In order to be a useful screening tool among healthy individuals, the determinants and distributional characteristics of a biomarker should be well-described. Disease association studies using vitamin D biomarkers with unaccounted-for variability has likely contributed to inconsistent findings and subsequently limited translation of meaningful results to the development of preventive interventions. In this study, it was reported that C24CPR has improved distributional characteristics among healthy individuals which may be useful in developing its use as a biomarker. Specifically, C24CPR was less skewed than 1,25(OH)₂D₃ for four of six population sub-groups, had a lower dispersion compared with a crude ratio of CYP24A1-competing dihydroxy metabolites, and more of the overall variability in C24CPR could be explained than for 1,25(OH)₂D₃. In addition, the observation that C24CPR values increased with greater WAGAP quartile, in light of the well-established observations of higher BMD (Wright N C et al., 2014, J Bone Miner Res, 29:2520-2526) and lower osteoporosis risk among African Americans (Jackson R D et al., 2014, Semin Reprod Med, 32:454-462), is at least one piece of evidence that indicates this biomarker might be useful. WAGAP was significantly associated with both 1,25(OH)₂D₃ and C24CPR, although explained less than 1% of variation in the former and ˜28% of the variability in the latter. All other genetic associations differed between C24CPR and 1,25(OH)₂D₃, which indicated that these biomarkers capture distinct aspects of vitamin D status. For example, three methionine-coding substitutions in separate genes were associated with higher C24CPR, but not 1,25(OH)₂D₃. Overall, the present results were consistent with previous observations that in order to be clinically relevant, vitamin D biomarkers need to account for genetic ancestry (LeFevre M L et al., 2015, Ann Intern Med, 162:133-140). The present results took this one step further, indicating the need to include other genetic variants.

A higher value of C24CPR describes a higher production of 1,25(OH)₂D₃, relative to its metabolic CYP24A1 competitors. Intracellular 1,25(OH)₂D₃ production of CYP27B1 has been mechanistically shown to be upregulated by inflammatory markers (IGF-1 and cyclic AMP response element binding protein (CREB)) (Geng S et al., 2011, Aging Cell, 10: 962-971). This was consistent with increased circulating 1,25(OH)₂D₃ concentrations observed in infection-related granulomatous disease (Tebben P J et al., 2016, Endocr Rev, 37:521-547). Similarly, very high C24CPR values may be deleterious and indicative of inflammation-mediated disease (Bosworth C et al., 2013, Semin Nephrol, 33:158-168; Muindi J R et al., 2013, Horm Cancer, 4:242-250). Published group-data biomarker concentrations were used to derive the C24CPR measure in inflammation-related patient populations, and found them to be 2- to 10-fold greater (chronic kidney disease=10.1; colon cancer, range 10.9 to 29.2) (Bosworth C et al., 2013, Semin Nephrol, 33:158-168; Muindi J R et al., 2013, Horm Cancer, 4:242-250) than the healthy EA in the present study. This indicated that both low and high values outside of a normal range are important, rather than a single cut-off value. Additional studies focus on the relationship between C24CPR, bone health, and inflammation.

### Vitamin D Binding Protein (Gc-Isoform) and Calbindin

The largest genetic differences in 1,25(OH)₂D₃ concentrations were in association with Gc-isoform and the GC×CALB1 interaction. GC knock-out animals have significantly lower circulating concentrations of both 25(OH)D₃ and 1,25(OH)₂D₃ (Safadi F F et al., 1999, J Clin Invest, 103:239-251). The present results were consistent with lower 1,25(OH)₂D concentrations among Gc-2 carriers (Lauridsen A L et al., 2005, Calcif Tissue Int, 77:15-22) possibly due to lower binding affinity of Gc-2 isoform (Arnaud J et al., 1993, Hum Genet, 92:183-138). Lower 25-hydroxyvitamin D metabolite concentrations among Gc-2 carriers have been observed among diverse population groups, including Caucasian (Pekkinen M et al., 2014, PLoS One, 9:e87292), African American (Wilson et al., 2018, Am J Epidemiol., 187:754-766) and Asian (Gozdzik A et al., 2011, J Steroid Biochem Mol Biol, 127:405-412) ancestry groups.

In contrast, a likely biological explanation for the GC×CALB1 interaction, which was observed herein, is not obvious. On its own, Calbindin-D28K plays a role in preventing calcium loss via calcium reabsorption, intracellular buffering and transport of intracellular calcium from the apical to the basolateral surfaces of the renal distal convoluted tubules of the kidney. CALB1 rs2142094^{p-502}, located in the promoter near a putative vitamin D response element (Gill R K et al., 1993, Proc Natl Acad Sci USA, 90:2984-2988), is in linkage disequilibrium with a transversion polymorphism CALB1 rs1800645^{p-366} that has been associated with renal cell cancer (Southard E B et al., 2012, Cancer Epidemiol Biomarkers Prev, 21:191-201), as well as rs201250370 located in a short-interspersed nuclear element (SINE). SINEs can influence gene transcription (Elbarbary R A et al., 2016, Science, 351:aac7247) and GWAS-identified SNPs are more likely to occur in SINE-enriched LD blocks (Payer L M et al., 2017, Proc Natl Acad Sci USA, 114:E3984-E3992). Finally, the influence of calbindin-D28K on circadian genes includes interaction with PDIA3, the intracellular vitamin D binding protein (Yuan G et al., 2017, J Bone Miner Res, 32:861-871). Parallel circadian variation of circulating 1,25(OH)₂D₃ and calbindin-D28K concentrations has been previously recognized (Miller B et al., 1982, FEBS Lett, 141:242-244). The associations that were observed were time of day adjusted, indicating an independent role of CALB1.

### CYP24A1, PPARGC1A and CYP27B1

CYP24A1 rs2762943 is located within a block of co-inherited variants previously associated with circulating 1,25(OH)₂D₃ (Wjst M et al., 2006, Respir Res, 7:60-70). CYP24A1 rs2762943 was significantly associated with C24CPR at Stage II but not in the final model—most likely because of the high D′ between this variant and CYP27B1 rs115631506, which remained associated with 1,25(OH)₂D₃ concentrations. PPARGC1A has been shown to increase mitochondrial CYP24A1 expression by as much as 60% and is an experimentally-confirmed co-activator of VDR (Savkur R S et al., 2005, Mol Pharmacol, 68:511-517). The present results were consistent with a hypothesis of increased stability of the PPARGC1A Ser⁴⁸² variant (Choi Y S et al., 2006, Biochem Biophys Res Commun, 344:708-712) leading to increased CYP24A1 expression and consequent lower 1,25(OH)₂D₃ concentrations. Taken together, genetic variation in CYP24A1 are likely to still be important in metabolism of 1,25(OH)₂D₃, in spite of the final stage model results. Larger studies are needed to identify interactions and tease apart the relationship between CYP24A1, CYP27B1, and PPARGC1A.

### CYP3A43 and CYP3A7

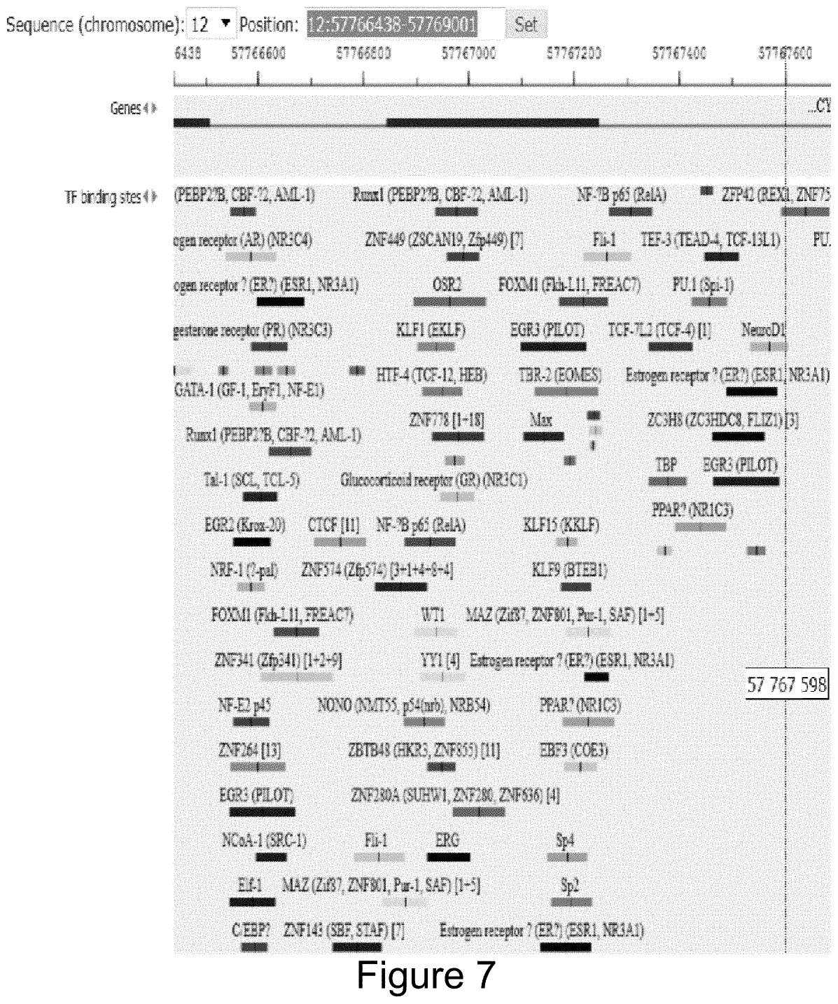

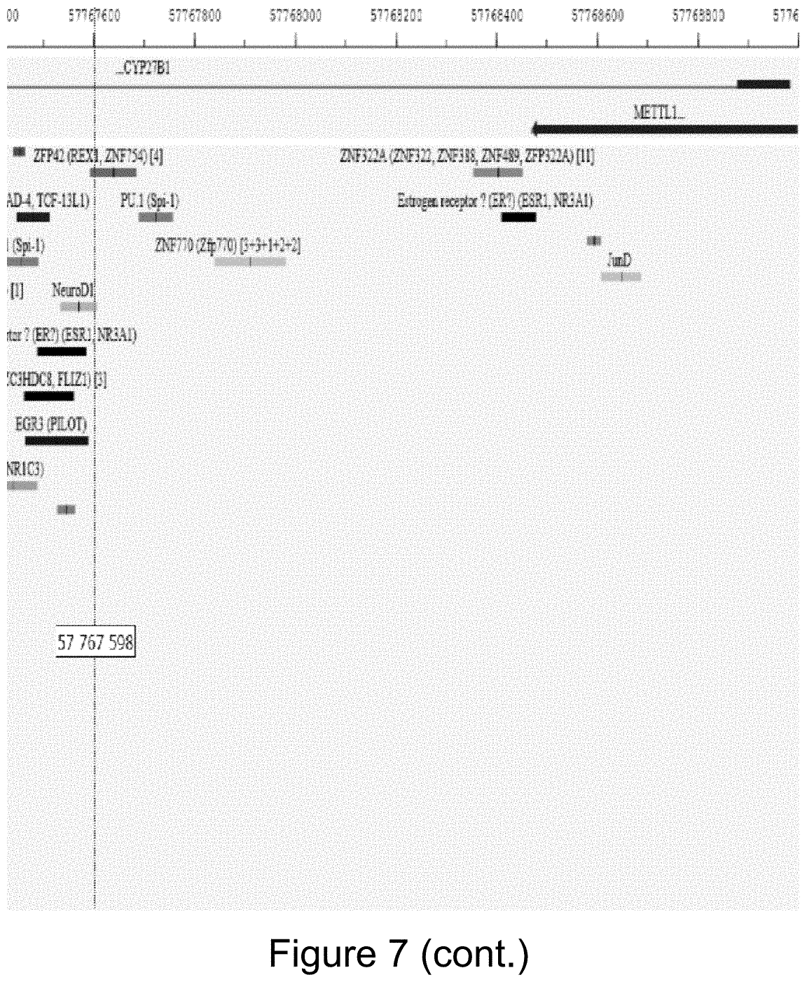

CYP3A43 and CYP3A7 are found in proximity on Chromosome 7. CYP3A7 is unique among the CYP3A family in that high hepatic expression at newborn only continues into adulthood for ˜10% of individuals. The adult expression phenotype is described by the CYP3A7*1C allele (Daly A K et al., 2006, Clinical pharmacokinetics, 45:13-31) and is determined by a seven SNP haplotype block (including CYP3A43 rs680055 and CYP3A7 rs2257401) and tagged by CYP3A7 rs45446698 (Wjst M et al., 2006, Respir Res, 7:60-70). The CYP3A7 rs2257401 variant occurs in a genomic region of high LD as well as structural variability in loss/gain of DNA (FIG. 7). The CYP3A7*1C allele is the consequence of a two base pair change in a pregnane-X-receptor DNA response element, which enables VDR to bind and up-regulate CYP3A7 expression (Hara H et al., 2004, Biochem Biophys Res Commun, 321(4):909-15). C24CPR was associated with genetic variation in CYP3A43, but not CYP3A7. The two SNPs, which were identified in separate models, were also partially linked—i.e., all minor allele carriers of CYP3A43 rs45621431 carry the major allele of CYP3A7 rs2257401, although not the inverse. This linkage provided evidence that the two biomarkers, that were investigated herein, are not completely genetically distinct. Currently, CYP3A4 is the only hepatic enzyme within the CYP3A family of enzymes that has been mechanistically demonstrated to metabolize vitamin D (Gupta R P et al., 2005, J Clin Endocrinol Metab, 90:1210-1219). These and the previous results (Roff A et al., 2008, Journal of Steroid Biochemistry & Molecular Biology, 112:47-54) highlight the need to further understand the functional characteristics of this portion of the genome with respect to vitamin D metabolism.

### CYP27B1, Klotho and FGF23

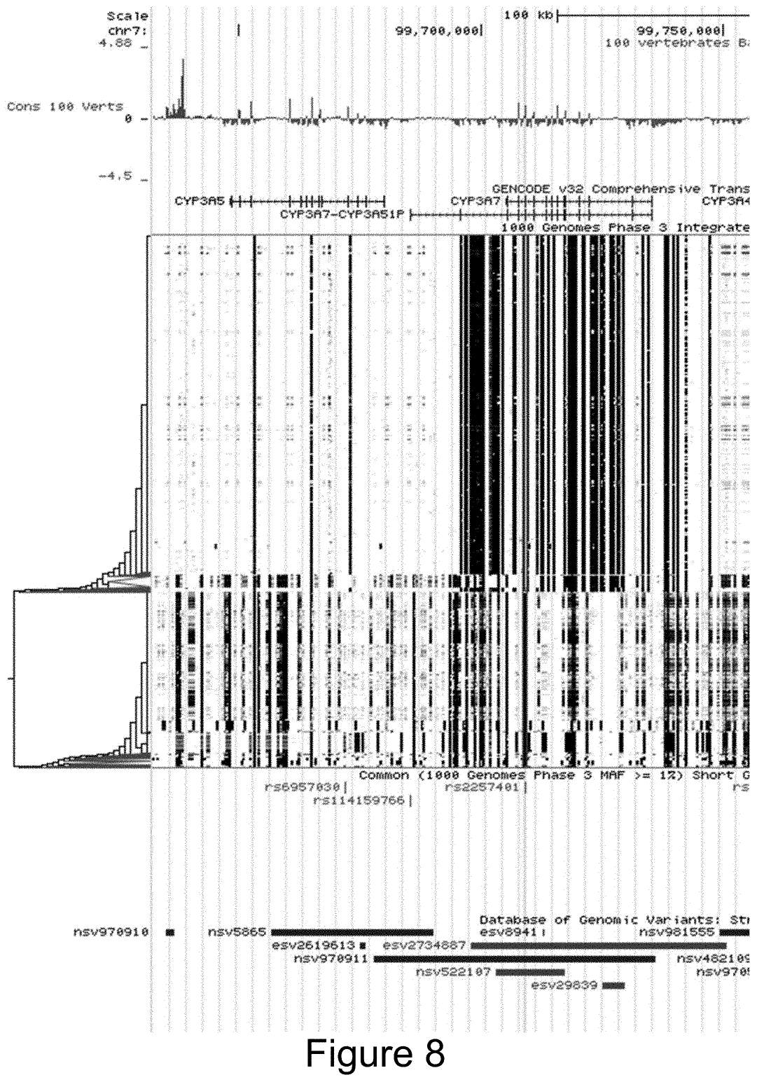

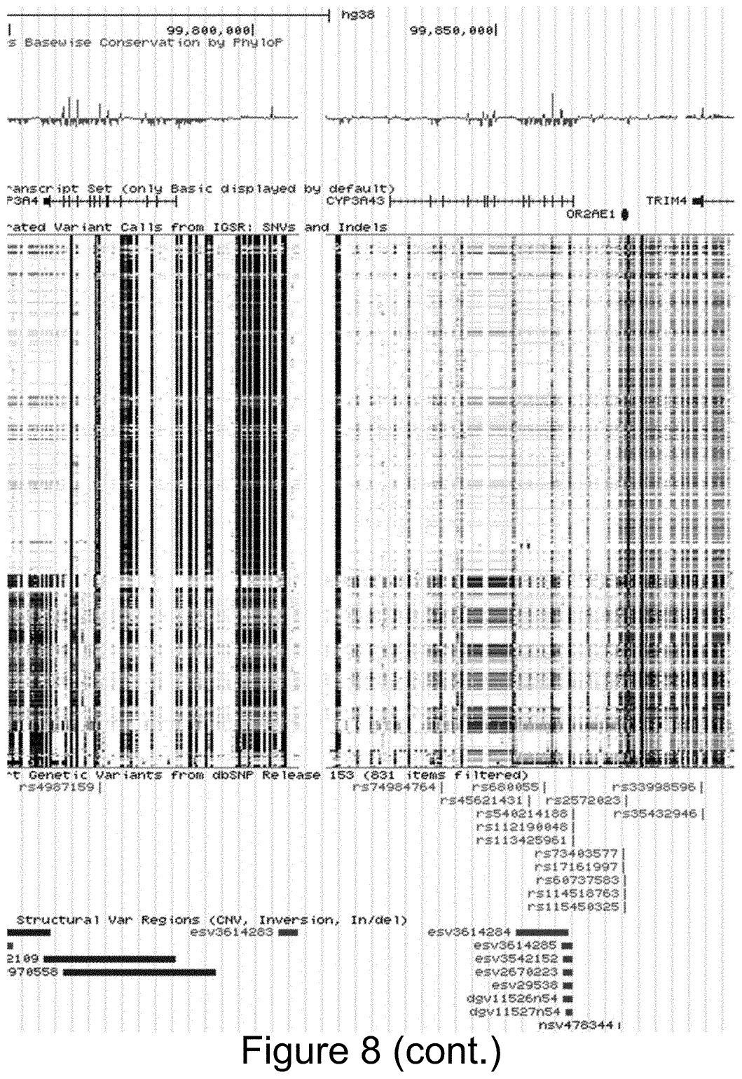

A 15 pmol/L difference was observed in circulating 1,25(OH)₂D₃ with respect to CYP27B1 rs115631506. While less than 10% carry the low 1,25(OH)₂D₃ allele, the magnitude of difference is similar to the effect of an oral 1,25(OH)₂D₃ dose of 0.5 ug/day (Barger-Lux M J et al., 1998, Osteoporos Int, 8:222-230). The rs115631506^{p-405} transversion polymorphism occurs at the 5^{th} base pair (from the 5′ end) of a mechanistically-confirmed negative vitamin D response element (nVDRE) (Turunen MINI et al., 2007, Nucleic Acids Res, 35:2734-2747). Compared with DNA transitions, DNA transversions are more likely to alter DNA shape, disrupt transcription factor binding, and have larger effects on gene expression (Guo C et al., 2017, BMC Genomics, 18:394). CYP27B1 rs115631506^{p-405} coincides with two other transcription factor binding sites (REX1 and NeuroD1 on chr12:58161381; FIG. 8). GWAS-identified variants in the distal chromosome 12 region (12q13.3-12q14.1) are hypothesized to influence DNA looping structure and alter CYP27B1 expression (Ramagopalan S V et al., 2011, Ann Neurol, 70:881-886; Turunen MINI et al., 2007, Nucleic Acids Res, 35:2734-2747).

Mechanistically, Klotho (encoded by the KL gene) inhibits CYP27B1 expression by binding to one of several FGF23 receptors. Consequently, KL- and FGF23-knockout mice exhibit high CYP27B1 kidney expression and elevated circulating 1,25(OH)₂D₃, with an advanced aging phenotype—i.e., increased atherosclerosis and osteoporosis (Yoshida T et al., 2002, Endocrinology, 143:683-689). Two studies report that KL rs9536314 (F³⁵²V) heterozygotes (whom had lowest C24CPR in this study) have higher overall survival, with lower cardiovascular disease and stroke in African American, Eastern European and Ashkenazi populations (Arking D et al., 2002, Proc Natl Acad Sci, 99:856-861; Arking D E et al., 2005, Circ. Res., 96:412-418). Conversely, FGF23 and Klotho can also induce CYP24A1 expression (Pike J W et al., 2017, Endocrinol Metab Clin North Am, 46:815-843). The magnitude of C24CPR difference that was observed for KL and FGF23 genotypes was ˜2-fold for some genotypes. The preponderance of methionine-coding changes in CYP3A43, KL and FGF23 associated with C24CPR also appear noteworthy. Methionine residues of proteins are more likely subject to age-related oxidation (Stadtman E R et al., 2005, Biochimica et Biophysica Acta, 1703:135-140) and may be possibly consistent with a high C24CPR because of protein modification as opposed to a functional genetic difference.

### Limitations

While the explained variability in circulating concentrations of 1,25(OH)₂D₃ that was reported is greater than previously published R² estimates (0.374 versus 0.180; (Engelman C D et al., 2008, J Clin Endocrinol Metab, 93:3381-3388)) substantial variability remains unexplained. It is also possible that metabolism of downstream metabolites may influence 1,25(OH)₂D₃ concentrations, but were not considered by this study (e.g., 1,24,25(OH)₃D₃, 24-oxo-1,25-dihydroxyvitamin D₃, 24-oxo-1,23,25-trihydroxyvitamin D₃, and 1,25(OH)₂-3-epi-D₃). Some of these downstream metabolites have been mechanistically shown to act like calcitriol (i.e., suppress PTH and/or promote intestinal calcium transport) and therefore may possibly influence the rate of 1,25(OH)₂D₃ production/catabolism (Lee N E et al., 1997, Biochemistry, 36:9429-9437). Finally, the results may not be generalizable to certain sub-groups—especially pregnant women, elders, and individuals with cancer or chronic liver or kidney disease.

### Strengths

This study was specifically designed to assess genetic differences in vitamin D metabolite concentrations and was conducted in non-summer months, reducing seasonal variability by design. Moreover, this is the first comprehensive analysis of genetic factors associated with 1,25(OH)₂D₃ concentrations determined by HPLC/MS-MS and not RIA. Unlike RIA assays, the HPLC/MS-MS assay separates vitamin D₂ and D₃ metabolite concentrations, allowing reduced variability due to dietary sources of D₂ as well as D₂/D₃ differences in side-chain metabolism (de Koning L et al., 2013, Clin Chim Acta, 415:54-58). The present results had several consistencies with previous studies reporting no association between circulating concentrations of 1,25(OH)₂D₃ and polymorphisms in VDR or CYP24A1 (Wishart J M et al., 1997, Am J Clin Nutr, 65:798-802; Hibler E A et al., 2010, J Steroid Biochem Mol Biol, 121:438-441; Hibler E A et al., 2015, Nutr Cancer, 67:1131-1141). The ancestry estimation method is highly concordant with other methods (Wilson R T et al., 2011, Hormone Molecular Biology and Clinical Investigation, 7:279-293). Average vitamin D intakes among participants were slightly above the US average of 232 to 264 IU/day for women and men in the United States ages 18 to 30, respectively—although still well below the recommended intake of 600 IU/day (ods.od.nih.gov/factsheets/vitamind-healthprofessional/). The lower median age of the study participants may have further reduced the likelihood of confounding by age-related factors, including occult chronic disease and/or chronic environmental exposures.

The present studies described key attributes of C24CPR, a novel measure of vitamin D status which is calculated as a function of three separately measured circulating vitamin D biomarkers. Theoretically, a higher value of C24CPR describes a higher production of calcitriol (1,25(OH)₂D₃) relative to its CYP24A1 competitors. C24CPR appears to be a distinct marker of vitamin D status in healthy young adults, based on the differing genetic and environmental associations that were observed herein. Compared with a crude ratio of metabolites, C24CPR may also have improved (less skewed) distributional characteristics. Unlike circulating concentrations of 25(OH)D₃ and 1,25(OH)₂D₃, C24CPR is lower among women whom are at a markedly higher risk of osteoporosis (FIG. 2). Similarly, in contrast to 25(OH)D₃ which is ˜2-fold higher among Caucasians, C24CPR is also lowest among those with high Western European (Caucasian)/low West African Genetic Ancestry proportion. Thus, two well-known risk factors for osteoporosis (female and Caucasian) are both associated with lower C24CPR, in contrast to circulating concentrations of 25(OH)D₃.

Taken together, these attributes suggest that the C24CPR biomarker would be suitable for normally-approximated expected value ranges that can be applied to populations according to genetic ancestry proportion (e.g, in personalized-prevention programs). Moving forward, ancestry-adjusted measures of vitamin D status and well-designed epidemiologic studies of biomarker-disease associations are warranted.

In summary, improved circulating biomarkers of vitamin D status are needed. CYP24A1 is the major vitamin D catabolic enzyme for which at least three major metabolites compete, including 25(OH)D₃, 24,25(OH)₂D₃, and the active form of vitamin D (1,25(OH)₂D₃). To compare genetic factors associated with circulating 1,25(OH)₂D₃ concentrations and the Calcitriol-24,25-Dihydroxyvitamin D3-Calcifediol Proportion Ratio (C24CPR) calculated as a function of 24,25(OH)₂D₃ and 25(OH)D₃. Higher C24CPR values describe a greater production of 1,25(OH)₂D₃ relative to 24,25(OH)₂D₃ and the upstream reserve of 25(OH)D₃.

Summarized Methods and Results of Differences Between C24CPR and 1,25(OH)₂D₃ Biomarkers. Using a cross-sectional study design, healthy young adults of African and European descent, matched (1:1) on age (+/−5 years) provided a blood sample in non-summer months (N=376). Vitamin D metabolites were measured in plasma with high performance liquid chromatography/tandem mass spectrometry (HPLC/MS-MS). West African genetic ancestry proportion (WAGAP) was estimated using STRUCTURE modeling of 112 autosomal genetic ancestry-informative markers. Multivariable mixed effect models estimated genetic associations with calcitriol and C24CPR, controlling for WAGAP, dietary vitamin D intake, sex, medication use and other environmental exposures. C24CPR values were not highly correlated with 1,25(OH)₂D₃ (rho=0.14). Genetic variants associated with C24CPR (CYP3A43, FGF23, KL, VDR) were not the same as those associated with 1,25(OH)₂D₃ (CALB1, CYP27B1, GC, PPARGC1A). WAGAP explained the largest proportion of variability in C24CPR (R²=0.282), but little variability in 1,25(OH)₂D₃ (R²=0.006). C24CPR values were positively associated with WAGAP (p-trend <0.001) and methionine-coding gene variants in VDR, CYP3A43 and FGF23. C24CPR is a distinct marker of vitamin D status in healthy young adults. Higher absolute values of C24CPR are consistent with a major determinant of higher bone mineral density—i.e., WAGAP.

### Example 3: C24CPR and Associated Health Outcomes

### I. Cancer

C24CPR is Associated with a Vitamin D Pathway Genetic Risk Score (VDGRS) a Potential Marker of Early Onset Renal Cell Cancer (eoRCC)
I. Cancer. Low C24CPR is significantly associated with higher values of a Vitamin D Pathway Genetic Risk Score (VDGRS). We also show that the VDGRS is significantly elevated in early onset renal cell cancer (eoRCC). A high number of VDGRS risk alleles is significantly associated with a low C24CPR value. As described in the following section I, C24CPR is the only vitamin D biomarker significantly associated with the VDGRS.

A new Vitamin D Pathway Genetic Risk Score (VDGRS) was developed and tested it in association with C24CPR and other vitamin D metabolites. Vitamin D-pathway genetic variants for the VDGRS were selected based on findings in published and unpublished works that were also confirmed in at least one additional study. Candidate variants for the VDGRS were those genetic variants either in association with circulating vitamin D metabolite concentrations (25(OH)D₃ or 1,25(OH)₂D₃) or with C24CPR (Wjst, 2006; Roff, 2008; Ahn 2010; Wilson, 2018; Wilson, unpublished C24CPR data). In some cases, variants were associated both with eoRCC (Karami, 2008) and C24CPR (Wilson, unpublished C24CPR data). C24CPR is significantly associated with the VDGRS. For each additional increasing number of VDGRS risk alleles, there is a significant decrease in the C24CPR (p=0.044). For each VDGRS number of risk alleles (range of risk alleles=0 to 5), the adjusted means for C24CPR in comparison to adjusted means for 25(OH)D₃ and adjusted means for 1,25(OH)₂D₃ are shown in Table 10.

TABLE 7
Association of Vitamin D Genetic Risk Score
(VDGRS) and Vitamin D Metabolites.

| | C24CPR | 25(OH)D₃ | 1,25(OH)₂D₃ |
|---|---|---|---|
| | Beta | Beta | Beta |
| Type of Statistical | Coefficient | Coefficient | Coefficient |
| Model | (p-value) | (p-value) | (p-value) |
| Bivariate* | −0.41 | 0.07 | −1.80 |
| | (0.050) | (0.978) | (0.309) |
| Multivariable | −0.37 | −0.61 | −0.92 |
| Adjustment** | (0.044)^{▴} | (0.748) | (0.581) |
| VDGRS Risk Allele | Adjusted Least Squares Means | | |
| Number | | | |
| 0 | 7.49 | 72.06 | 99.53 |
| 1 | 7.49 | 69.95 | 96.97 |
| 2 | 6.61 | 71.92 | 95.24 |
| 3 | 6.49 | 70.29 | 96.48 |
| 4 | 6.98 | 62.60 | 102.90 |
| 5 | 4.94 | 82.36 | 62.031 |
| p−trend | (0.050)^{▴} | (0.757) | (0.529) |

*Pearson Correlation Coefficient;
**Linear Regression, adjusting for age, body mass index, number of days from summer solstice, sex, systolic blood pressure, tanning bed use, time of day, vitamin D intake (from diet, supplements and multi-vitamins), West African Ancestry proportion.
***Adjusted Least Squares Means, adjusted for all variables above as in (**);
^{▴}This association is statistically significant at p-value ≤0.050.

Association Between VDGRS, eoRCC Case Status and DNA Damage.

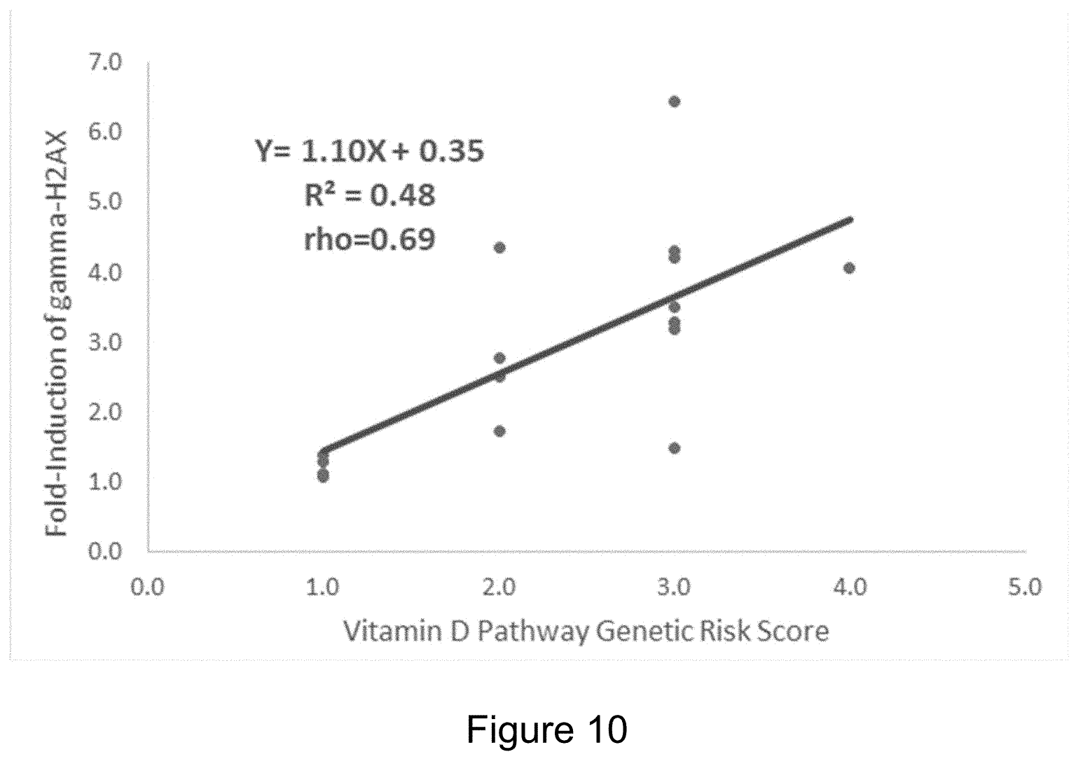

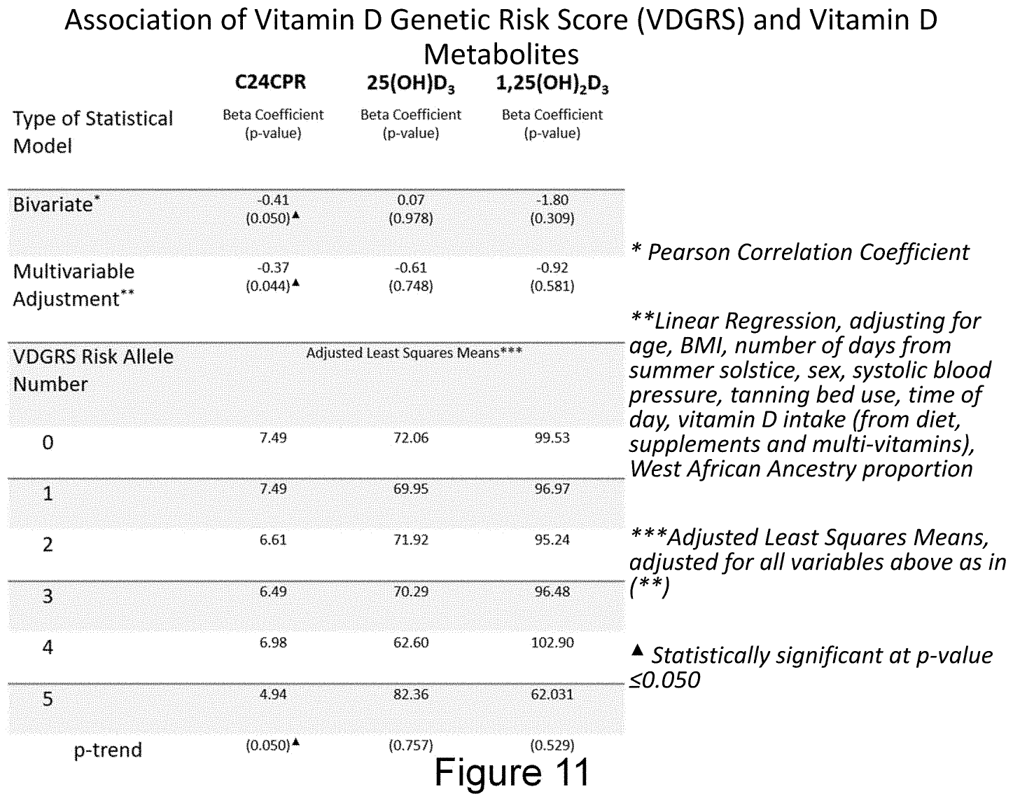

The VDGRS was tested among eoRCC cases referred for clinical genetics screening within the Temple Fox Chase Cancer Center's Clinical Risk Assessment Program, 22 individuals screened for family cancer syndromes and having no clinically-identified germline pathogenic variants/family cancer syndrome and reporting at least one relative with cancer, were selected at random. Germline whole exome sequencing (WES) and targeted confirmatory genotyping of VDGRS variants was conducted by laboratory staff blinded to the case/control status and biomarker status of participants. Among eoRCC cases in this investigation, 73% (n=16/22) had a family history of RCC, with 50% (n=11/22) having a first-degree relative with RCC. The mean VDGRS was significantly higher among eoRCC compared with healthy controls matched by race/ethnicity from an earlier study of vitamin D metabolism (mean VDGRS=2.3 in eoRCC versus 1.5 in healthy controls, respectively, Wilcoxon Rank Sum test p-value=0.002). It has been previously recognized that, γ-H2AX (aka gamma-H2AX, a DNA-damage marker) is elevated among eoRCC, compared with age- and sex-matched healthy controls (p<0.001; (Demidova, 2022). In addition, the VDGRS exhibited a moderately high linear correlation (rho=0.69) between the VDGRS and in vitro fold-induction of DNA damage (aphidicolin-induced γ-H2AX) in eoRCC patient lymphocytes (FIG. 10). This correlation was stronger for the VDGRS than for any single vitamin D-pathway variant of which it was comprised (i.e., all individual rho equal to or less than 0.35). In summary, C24CPR is associated with VDGRS, which, in turn, is enriched in eoRCC patients and has a moderately high correlation with a biomarker of DNA damage, γ-H2AX.

### II. Hypertension

High C24CPR is significantly associated with a 3.8-fold risk of hypertension among young adult women. This relationship increased to a 27-fold risk among young adult women taking oral contraceptives. As described in the following section II, C24CPR could be used in prediction and biological feedback in hypertension management among young adults.

### Potential Use of C24CPR as Biological Feedback in Hypertension Diagnosis Among Young Adult Women.

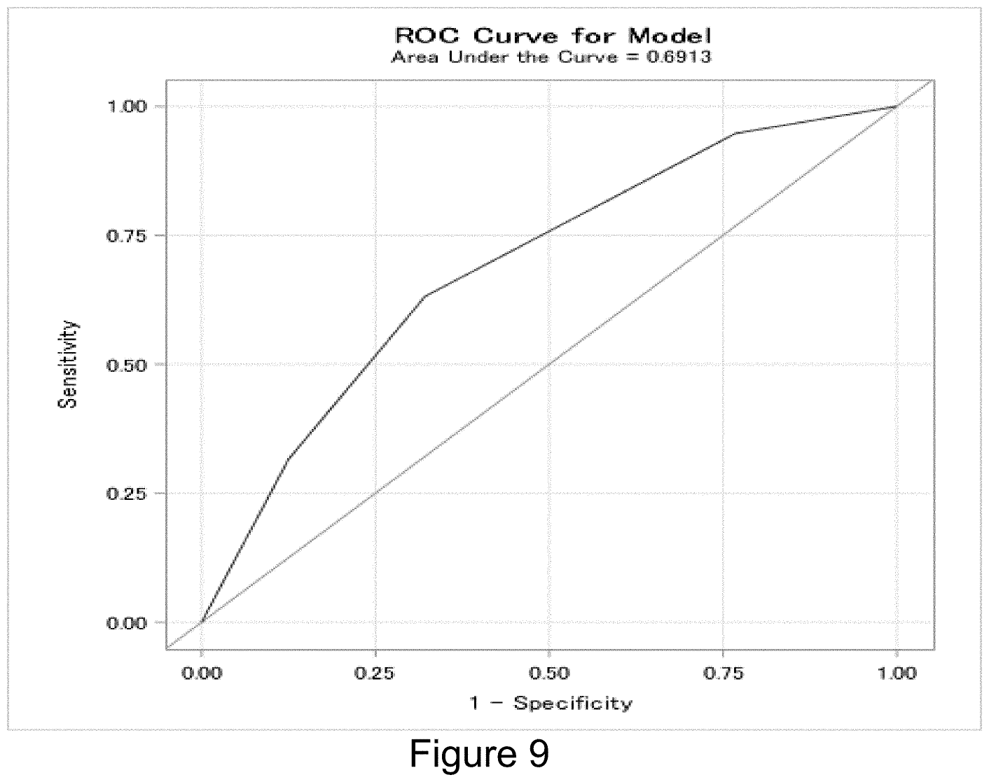

Among young adults (ages 18 to 35), C24CPR concentrations were divided into ancestry-specific quartiles. Highest values of C24CPR were associated with a 3.84-fold increase in risk of hypertension among women (aOR=3.84, 95% CI: 1.03-14.31, p=0.045, no oral contraceptive use). Among women taking oral contraceptives, the highest C24CPR quartile was associated with a 27-fold risk of hypertension (aOR=27.06, p=0.022). A receiver-operating characteristic (ROC) curve analysis of the C24CPR biomarker was used to determine the area under the curve using the high-risk cut-off values for women taking oral contraceptives. The area under the curve (AUC) of the ROC curve analysis was 0.6913, FIG. 9. This value is near the acceptable range of viable biomarkers, generally considered to be 0.70 or greater. In this study, a high proportion of young adults were unaware of their hypertension status and nearly all were not receiving treatment (Cuffee, 2021). Combined with traditional in-clinic methods of hypertension assessment, patient knowledge of biomarker values (“biological feedback”) may help underscore the need for health-behavior improvements and increase health seeking behavior in patients (Richardson, 2022).

TABLE 8
Risk of Hypertension Versus Normotension by Vitamin D Biomarkers levels
(25(OH)D₃, 1,25(OH)₂D₃ and 24,25(OH)₂D₃ and C24CPR) among Young Adults
(Ages 18 to 35), Centre County Pennsylvania, 2006-2016.

| | | | | H-L Model |
|---|---|---|---|---|
| | Adjusted Odds Ratio | 95% Confidence | Parameter | Goodness of |
| Biomarker quartile | (aOR)* | Interval | P-value | Fit P-value |
| C24CPR (ratio, ancestry-specific), | | | | |
| men | | | | |
| Ancestry-specific Quartile 1 | 0.76 | 0.22-2.70 | 0.690 | |
| Ancestry-specific Quartile 2 | Reference | | | |
| Ancestry-specific Quartile 3 | 0.51 | 0.16-1.65 | 0.263 | |
| Ancestry-specific Quartile 4 | 0.93 | 0.33-2.60 | 0.894 | 0.159 |
| p-trend (order Q2, Q1, Q3, Q4) | 0.924 | | | |
| C24CPR (ratio, ancestry-specific), | | | | |
| women (no OC use) | | | | |
| Ancestry-specific Quartile 1 | 3.80 | 0.94-15.32 | 0.061 | |
| Ancestry-specific Quartile 2 | Reference | | | |
| Ancestry-specific Quartile 3 | 1.99 | 0.46-8.66 | 0.358 | |
| Ancestry-specific Quartile 4 | 3.84 | 1.03-14.31 | 0.045 | 0.229 |
| p-trend (order Q2, Q1, Q3, Q4) | 0.111 | | | |
| C24CPR (ratio, ancestry-specific), | | | | |
| women (current OC use) | | | | |
| Ancestry-specific Quartile 1 | 5.47 | 0.43-70.23 | 0.192 | |
| Ancestry-specific Quartile 2 | Reference | | | |
| Ancestry-specific Quartile 3 | 5.64 | 0.46-69.38 | 0.177 | |
| Ancestry-specific Quartile 4 | 27.06 | 1.62-452.97 | 0.022 | 0.389 |
| p-trend (order Q2, Q1, Q3, Q4) | 0.022 | | | |

*All Odds Ratios adjusted for age (continuous), sex (binary), smoking (former, current, none), West African genetic ancestry proportion (continuous), obesity (WHO classification: underweight, normal weight, overweight, obese), and self-reported physical activity in the past 30 days (none, monthly, 1 to 3 times/week, 4 to 6 times per week, daily).
All biomarker quartile cut offs classified according to quartiles among normotensive individuals.
Ancestry-specific quartiles determined in individuals with West African genetic ancestry greater than 0.50 and in individuals with West African genetic ancestry less than or equal to 0.50.
H-L = Hosmer-Lemeshow.
P-trend values based on quartile values 1 through 4 with rank ordering shifted for ancestry-specific trends as indicated.
All analyses conducted in SAS version 9.4 using SAS Proc Logistic.
Bolded p-values are statistically significant at <0.05 for aORs.
H-L p-values below 0.100 reject the null hypothesis of goodness of fit, suggesting model lack of fit.
OC use = oral contraceptive use.
Ancestry-specific quartiles for West African genetic ancestry proportion >0.50 (<6.51, 6.51-<8.75, 8.75-12.02, 12.02+) and ancestry-specific quartiles for West African genetic ancestry proportion ≤0.50 (<3.27, 3.27-<4.43, 4.42-<5.80, 5.80+), calculated among 238 normotensive individuals.

In summary, the present data provides information regarding the proposed Calcitriol-24,25-Dihydroxyvitamin D3-Calcifediol Proportion ratio (C24CPR) biomarker. Specifically, using ancestry-specific cut off values, high C24CPR values indicate hypertension in young adults. Conversely, low C24CPR values indicate the risk of early onset renal cell cancer.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A method of distinguishing a subject with a disease or disorder associated with vitamin D deficiency or vitamin D imbalance from a subject without disease or disorder associated with vitamin D deficiency or vitamin D imbalance, the method comprising:
a. obtaining a biological sample from a test subject;
b. analyzing the biological sample with an assay that specifically detects at least one biomarker, wherein the at least one biomarker is calcitriol to 24,25-dihydroxyvitamin D to calcifediol proportion ratio (C24CPR);
c. detecting the level of the at least one biomarker in the biological sample of the subject;
d. comparing the level of the at least one biomarker in the biological sample to a comparator of the at least one biomarker;
e. determining that the subject has the disease or disorder associated with vitamin D deficiency or vitamin D imbalance when the at least one biomarker is differentially expressed in the biological sample as compared to the comparator; and
f. administering a treatment of the disease or disorder associated with vitamin D deficiency or vitamin D imbalance to the subject determined to have the disease or disorder associated with vitamin D deficiency or vitamin D imbalance.

2. The method of claim 1, wherein the disease or disorder associated with vitamin D deficiency or vitamin D imbalance is selected from the group consisting of: a cancer, autoimmune disease or disorder, diabetes, adenoma, metabolic syndrome, chronic pain, decline in renal function, and any combination thereof.

3. The method of claim 2, wherein the disease or disorder associated with vitamin D deficiency or vitamin D imbalance is selected from the group consisting of: a renal cell cancer, prostate cancer, colon cancer, colorectal cancer, colorectal adenoma, type 2 diabetes, or any combination thereof.

4. The method of claim 1, wherein the biological sample comprises a biological tissue of the subject, a blood sample of the subject, a bodily fluid sample of the subject, a fecal sample of the subject, a plasma sample of the subject, a saliva sample of the subject, a urine sample of the subject, and any combination thereof.

5. The method of claim 1, wherein the assay is selected from the group consisting of: mass spectrometry (MS), liquid chromatography (LC), liquid chromatography-mass spectrometry (LC-MS), targeted liquid chromatography-tandem mass spectrometry (LC-MS/MS), high-performance liquid chromatography (HPLC), ultra-performance liquid chromatography (UPLC), ultra-high-performance liquid chromatography (UHPLC), gas chromatography (GC), gas chromatography-mass spectrometry (GC-MS), globally optimized targeted mass spectrometry, targeted assay of about 200 metabolites, aqueous global profiling, liquid global profiling, GC-MS profiling, GC-MS flux analysis, carnitine analysis, lipid targeted analysis, quantitative lipid targeted analysis, tryptophan analysis, absolute quantification, multivariate statistical analysis, dynamic light scattering (DLS), nuclear magnetic resonance (NMR) spectroscopy, ultraviolet-visible (UV/Vis) spectroscopy, infrared (IR) spectroscopy, Raman spectroscopy, and any combination thereof.

6. The method of claim 1, wherein the method comprises using a multi-dimensional non-linear algorithm to determine if the level of a set of biomarkers in the biological sample is statistically different as compared to the comparator.

7. The method of claim 1, wherein the subject is determined to have disease or disorder associated with vitamin D deficiency or vitamin D imbalance when the level of the at least one biomarker in the biological sample is decreased as compared to the comparator.

8. The method of claim 1, wherein the treatment is determined based on the level of the at least one biomarker in a subject.

9. The method of claim 1, wherein the treatment comprises administering a therapeutically effective amount of at least one selected from the group consisting of agonist of calcitriol, agonist of 24,25-dihydroxyvitamin D, agonist of calcifediol, antagonist of calcitriol, antagonist of 24,25-dihydroxyvitamin D, antagonist of calcifediol, and any combination thereof.

10. The method of claim 1, wherein the subject has at least one genetic variation associated with vitamin D metabolite.

11. The method of claim 10, wherein the at least one genetic variation associated with vitamin D metabolite is selected from the group consisting of one or more variations in CYP3A43, one or more variations in FGF23, one or more variations in KL, one or more variations in VDR, one or more variations in CALB1, one or more variations in CYP27B1, one or more variations in GC, one or more variations in PPARGC1A, and any combination thereof.

12. The method of claim 11, wherein the at least one genetic variation associated with vitamin D metabolite is selected from the group consisting of one or more variations in CYP3A43, one or more variations in FGF23, one or more variations in KL, one or more variations in VDR, and any combination thereof.

13. A method of preventing a disease or disorder associated with vitamin D deficiency or vitamin D imbalance in a subject in need thereof, the method comprising:
a. obtaining a biological sample from a test subject;
b. analyzing the biological sample with an assay that specifically detects at least one biomarker, wherein the at least one biomarker is calcitriol to 24,25-dihydroxyvitamin D to calcifediol proportion ratio (C24CPR);
c. detecting the level of the at least one biomarker in the biological sample of the subject;
d. comparing the level of the at least one biomarker in the biological sample to a comparator of the at least one biomarker; and
e. determining that the subject is at risk of developing the disease or disorder associated with vitamin D deficiency or vitamin D imbalance when the at least one biomarker is differentially expressed in the biological sample as compared to the comparator.

14. The method of claim 13, wherein the method further comprises
f. administering a treatment of the disease or disorder associated with vitamin D deficiency or vitamin D imbalance to the subject.

15. The method of claim 14, wherein the method is a method of preventing a disease or disorder associated with vitamin D deficiency or vitamin D imbalance.

16. The method of claim 14, wherein the treatment is adjusted based on the level of the at least one biomarker in a subject.

17. The method of claim 16, wherein the subject is a woman.

18. A method of assessing a risk of hypertension or detecting a vitamin D deficiency or vitamin D imbalance in a subject, the method comprising:
a. obtaining a biological sample from a test subject;
b. analyzing the biological sample with an assay that specifically detects at least one biomarker, wherein the at least one biomarker is calcitriol to 24,25-dihydroxyvitamin D to calcifediol proportion ratio (C24CPR);
c. detecting the level of the at least one biomarker in the biological sample of the subject;
d. comparing the level of the at least one biomarker in the biological sample to a comparator of the at least one biomarker; and
e. determining that the subject is at a risk of hypertension or has vitamin D deficiency or vitamin D imbalance when the at least one biomarker is differentially expressed in the biological sample as compared to the comparator.

19. The method of claim 18, wherein the subject is a woman.

20. The method of claim 18, wherein the method further comprises administering a treatment for hypertension to the subject.
